(19) United States
(12) Patent Application Publication
(10) Pub. No.: US 2026/0108243 A1
(43) Pub. Date: Apr. 23, 2026

(54) METHOD AND APPARATUS FOR PASSING SUTURE

(71) Applicant: Clauson Creative Engineering, Inc., Reno, NV (US)

(72) Inventors: Brett A. SNYDER, Reno, NV (US); Andrew J. HUNTINGTON, Reno, NV (US); Ryan N. SCHMID, Reno, NV (US); Yasuyuki HITOMI, Kyoto (JP); Harry NGUYEN, Westminster, CA (US); John L. MINCK, Jr., Reno, NV (US)

(73) Assignee: Clauson Creative Engineering, Inc., Reno, NV (US)

(21) Appl. No.: 19/427,917

(22) Filed: Dec. 19, 2025

Related U.S. Application Data
(63) Continuation of application No. 17/446,259, filed on Aug. 27, 2021.

(51) Int. Cl. A61B 17/04; A61B 17/00; A61B 17/29

(52) U.S. Cl. CPC A61B 17/0491; A61B 2017/00367; A61B 2017/047; A61B 2017/2925; A61B 2017/2946

(57) ABSTRACT

A device is disclosed that can pierce and hold tissue and then pass suture through tissue. The device can have a shuttle that can removably attach to a suture and jaws that can be opened and closed. A method for using the device to repeatedly pass the suture through the tissue with or without removing the suture or device from the target site is disclosed.

### BACKGROUND

### 1. Technical Field

This disclosure relates to system, methods, and apparatus for enhancing the advancement and retention of suture through tissue.

### 2. Background

Suturing apparatus in the past have had a shaft and a distal clamping mechanism to facilitate their use through cannulas in less invasive surgery. These devices have typically included opposing jaws which clamp onto the tissue to be sutured. The end segment of the suture is pre-positioned and secured at the distal end of one jaw member.

It would be advantageous to have an apparatus that could load and unload suture without the need to remove the apparatus from the surgical site.

It would be advantageous to have an apparatus that could pass (not load and unload) suture repeatedly through tissue without the need to remove the apparatus from the surgical site. It would also be advantageous for the suture shuttling mechanism (either needle or shuttle) to be entirely contained within the apparatus during operation to improve accuracy of suture placement and improve safety of needle or shuttle position during operation.

A need exists for the features disclosed herein, including, for example, improved suturing controls and improved shuttle retention (passive and/or active).

### SUMMARY

This disclosure relates generally to suture devices and methods of suturing.

A method of suturing tissue with a suture passer is disclosed. The suture passer can have a first jaw, a second jaw, a first trigger, a second trigger, and a selector. The method can include selecting a first pusher with the selector. The method can include moving the first jaw toward the second jaw with the first trigger. The method can include moving the first pusher from a first pusher first position to a first pusher second position with the second trigger to move the shuttle from the first jaw to the second jaw. The method can include moving the first pusher from the first pusher second position to the first pusher first position with the second trigger. The method can include moving the first jaw away from the second jaw with the first trigger.

A suture passer is disclosed. The suture passer can have a lockable and unlockable mover. The suture passer can have a first jaw. The first jaw can have a first jaw track and a first jaw first stop. The suture passer can have a shuttle. The shuttle can have a shuttle longitudinal axis and a shuttle first stop. The shuttle can be movable in the first jaw track. The shuttle first stop can extend away from the shuttle longitudinal axis. The shuttle first stop can be movable into and out of the first jaw first stop. When the shuttle first stop is in the first jaw first stop, the shuttle can be retained in the first jaw.

A suture passer is disclosed. The suture passer can have a first jaw and a shuttle. The first jaw can have a first jaw track, a first jaw first stop, and a first jaw second stop. The shuttle can have a shuttle longitudinal axis and a shuttle first stop. The shuttle can be movable in the first jaw track. The shuttle first stop and the first jaw first stop can be engageable with each other. The shuttle first stop and the first jaw second stop can be engageable with each other.

When the shuttle first stop and the first jaw first stop are engaged with each other, the shuttle can be retainable in the first jaw. When the shuttle first stop and the first jaw second stop are engaged with each other, the shuttle can be retainable in the first jaw.

### BRIEF DESCRIPTION OF THE FIGURES

The drawings shown and described are exemplary variations and non-limiting. Like reference numerals indicate identical or functionally equivalent features throughout.

FIG. 1 is a variation of a perspective view of a variation of the device.

FIGS. 2A-2E are variations of a side view of the device of FIG. 1 with a selector and half of the handle shown transparent and with features of the device in various configurations.

FIGS. 3A-3E are variations of a side view of the device of FIG. 1 with a selector and half of the handle shown transparent and with the features of the device in various configurations.

FIGS. 4A-4E show FIGS. 2A-2E with the selector.

FIGS. 5A-5E show FIGS. 3A-3E with the selector.

FIGS. 6A-6E show FIGS. 2A-2E with the selector and the handle.

FIGS. 7A-7E show FIGS. 3A-3E with the selector and the handle.

FIG. 8 illustrates a magnified view of FIG. 2A at section 8-8.

FIGS. 9A-9E illustrate various features of the device and various configurations of the device of FIG. 1.

FIG. 9F illustrates a variation of a mover of the device of FIGS. 9A-9E.

FIGS. 10A-10C illustrate various features of the device and various configurations of the device of FIG. 1.

FIG. 11 illustrates a variation of a perspective view of the device of FIG. 1.

FIG. 12 illustrates a variation of a perspective view of the device of FIG. 1.

FIG. 13A illustrates a variation of a perspective view of a mover of the device of FIG. 1.

FIG. 13B illustrates a variation of a perspective view of a mover of the device of FIG. 1.

FIG. 13C illustrates various features of the device and a variation of a configuration of the device of FIG. 1.

FIGS. 14A-14H illustrate various features of the device and various configurations of the device of FIG. 1, with FIG. 14A illustrating a variation of a center longitudinal cross-section of a distal end of the device of FIG. 1 taken along the line 14A-14A in FIG. 2B.

FIG. 14I illustrates a variation of the device of FIG. 1.

FIG. 14J illustrates a variation of the device of FIG. 1.

FIG. 14K illustrates a variation of the device of FIG. 1.

FIGS. 15A and 15B illustrate variations of the shuttle.

FIG. 16A illustrates a variation of the shuttle in a jaw with half the jaw shown transparent.

FIG. 16B illustrates a variation of the shuttle in a jaw with half the jaw shown transparent.

FIG. 17A illustrates a variation of the shuttle in a jaw with half the jaw shown transparent.

FIG. 17B illustrates a variation of the shuttle in a jaw with half the jaw shown transparent.

FIG. 18 illustrates a variation of the device with half the jaws shown transparent.

FIG. 19A illustrates a perspective view of a variation of a shuttle.

FIG. 19B illustrates a bottom view of the shuttle of FIG. 19A.

FIG. 19C illustrates a side view of the shuttle of FIG. 19A.

FIG. 20A illustrates a top view of a variation of a shuttle in a flat configuration.

FIG. 20B illustrates a top view of a variation of the shuttle of FIG. 20A and a variation of a pusher in a jaw track with the jaw track shown transparent.

FIG. 20C illustrates a top view of a variation of the shuttle of FIG. 20A and a variation of a pusher in a jaw track with the jaw track shown transparent.

FIG. 20D illustrates a top view of a variation of the shuttle of FIG. 20A and a variation of a pusher in a jaw track with a top of the jaw shown transparent.

FIG. 20E illustrates a top view of a variation of the shuttle of FIG. 20A and a variation of a pusher in a jaw track with a top of the jaw shown transparent.

FIG. 20F illustrates a perspective view of a variation of a jaw with half of the jaw shown transparent.

FIG. 20G illustrates a perspective view of the jaw of FIG. 20F with a variation of a shuttle and with half of the jaw shown transparent.

FIG. 20H illustrates a perspective view of a variation of a jaw with half of the jaw shown transparent.

FIG. 20I illustrates a perspective view of the jaw of FIG. 20I with a variation of a shuttle and with half of the jaw shown transparent.

FIG. 21A illustrates a front perspective view of a variation of a shuttle.

FIG. 21B illustrates a rear perspective view of the shuttle of FIG. 21A

FIG. 21C illustrates a front perspective view of a variation of a shuttle.

FIG. 21D illustrates a rear perspective view of the shuttle of FIG. 21C.

FIGS. 22A and 22B each illustrate a top view of a variation of the shuttle and a first pusher in a first track with a portion of the first jaw shown transparent.

FIGS. 23A and 23B each illustrate a top view of a variation of the shuttle and a second pusher in a second track with a portion of the second jaw shown transparent.

FIGS. 24A and 24B illustrate variations of perspective views of the device of FIG. 1 having multiple female stops in a first jaw.

FIGS. 24C and 24D illustrate variations of perspective views of the device of FIG. 1 having multiple female stops in a second jaw.

FIG. 25A illustrates a variation of a perspective view of the device of FIG. 1.

FIG. 25B illustrates a variation of a perspective view of the device of FIG. 1.

FIG. 26A illustrates a variation of a perspective view of the device of FIG. 1.

FIG. 26B illustrates a variation of a perspective view of the device of FIG. 1.

FIG. 26C illustrates a variation of a perspective view of the device of FIG. 1.

FIG. 26D illustrates a variation of a perspective view of the device of FIG. 1.

FIG. 26E illustrates a variation of a locked configuration of the device of FIG. 1.

FIG. 26F illustrates a variation of a locked configuration of the device of FIG. 1.

FIG. 26G illustrates a variation of a configuration of the device of FIG. 1.

FIG. 26H illustrates a variation of a configuration of the device of FIG. 1.

FIG. 27A illustrates a variation of a configuration of the device of FIG. 1.

FIG. 27B illustrates a variation of a configuration of the device of FIG. 1.

FIGS. 28A and 28B each illustrate a variation of a perspective view of the shuttle of the device.

FIG. 29A illustrates a variation of a perspective view of an exemplary configuration of the device.

FIG. 29B illustrates a variation of a perspective view of an exemplary configuration of the device.

FIG. 30A illustrates a variation of a perspective view of a first pusher.

FIG. 30B illustrates a magnified view of FIG. 30A at section 30B-30B.

FIG. 30C illustrates a variation of a perspective view of a first pusher

FIG. 30D illustrates a magnified view of FIG. 30A at section 30D-30D.

FIG. 30E illustrates a variation of a side view of the device of FIG. 1 with half of the device shown transparent.

FIG. 31A illustrates a variation of a side view of a variation of the device of FIG. 1.

FIG. 31B illustrates a variation of a top view of the device of 31A.

FIG. 31C illustrates a magnified view of FIG. 31A at section 31C-31C.

FIG. 31D illustrates a variation of the features shown in FIGS. 31A-31C.

FIG. 31E illustrates a magnified view of FIG. 31A at section 31E-31E.

FIG. 31F₁-31F₃ illustrate magnified views of three configurations of the lockout bar in FIG. 31A at section 31F-31F.

### DETAILED DESCRIPTION

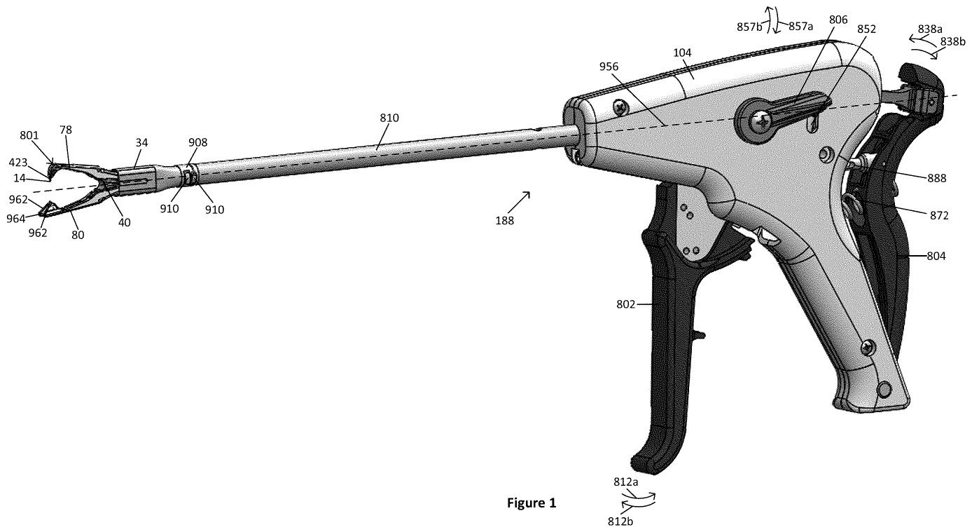

The features and methods described herein and/or illustrated in FIGS. 1-31F₃ can be combined with each other in any combination. U.S. application Ser. No. 16/733,740 (US Publication No. 2021/0204934) filed Jan. 3, 2020 and U.S. application Ser. No. 16/734,406 (US Publication No. 2021/0204935) filed Jan. 6, 2020 are herein incorporated by reference in their entirety for all purposes. Any of the features and methods described and/or illustrated in these two applications incorporated by reference can be combined in any combination with the present disclosure.

FIGS. 1-3E illustrate a variation of a suture passer 188 in various configurations. The suture passer 188 is also referred to as the device 188.

FIGS. 1-3E illustrate that the device 188 can have a first jaw 78 and a second jaw 80. The first and second jaws 78, 80 are also referred to collectively as the jaws 801. The jaws 801 can be openable and closable. One or both of the jaws 801 can be movable. For example, FIGS. 1-3E illustrate that both of the jaws 801 can be movable. As another example, one of the jaws 801 can be a stationary jaw and the other one of the jaws 801 can be a movable jaw.

FIGS. 1-3E illustrate that the device 188 can have a first pusher 76 and a second pusher 86. The first and second pushers 76, 86 are also referred to collectively as the pushers 803. The pushers 803 can be made of, for example, Nitinol.

FIGS. 1-3E illustrate that the device 188 can have a shuttle 14. The shuttle 14 can be moved (e.g., pushed) back and forth between the jaws 801 via the pushers 803. For example, FIGS. 1-3E illustrate that the first pusher 76 can be push the shuttle 14 from the first jaw 78 to the second jaw 80 and that the second pusher 86 can push the shuttle 14 from the second jaw 80 to the first jaw 78.

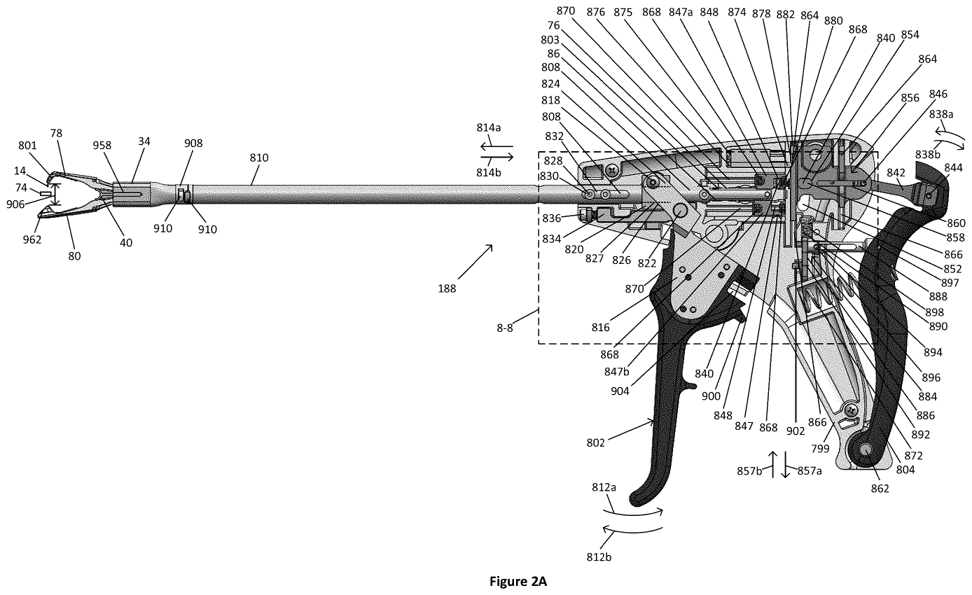

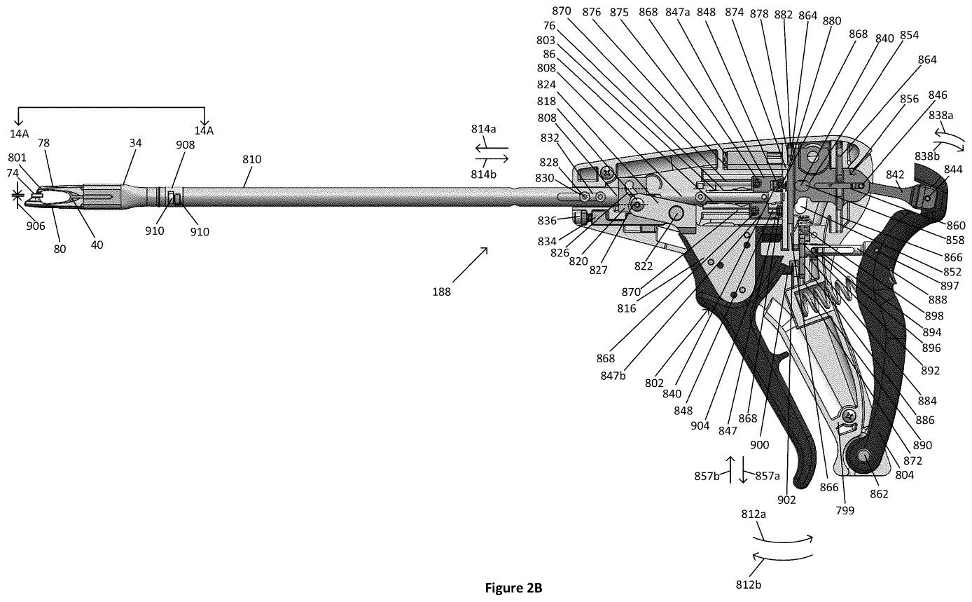

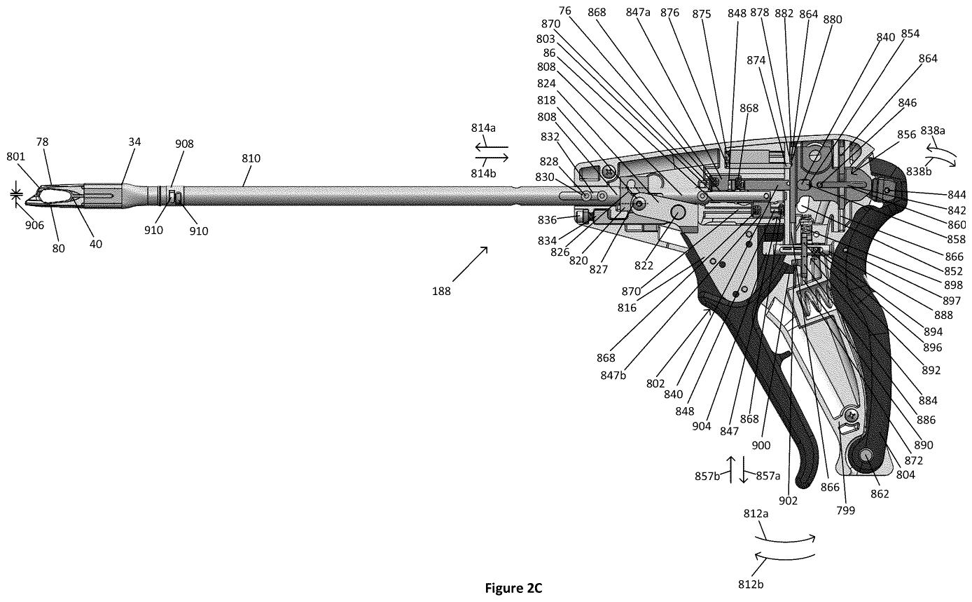

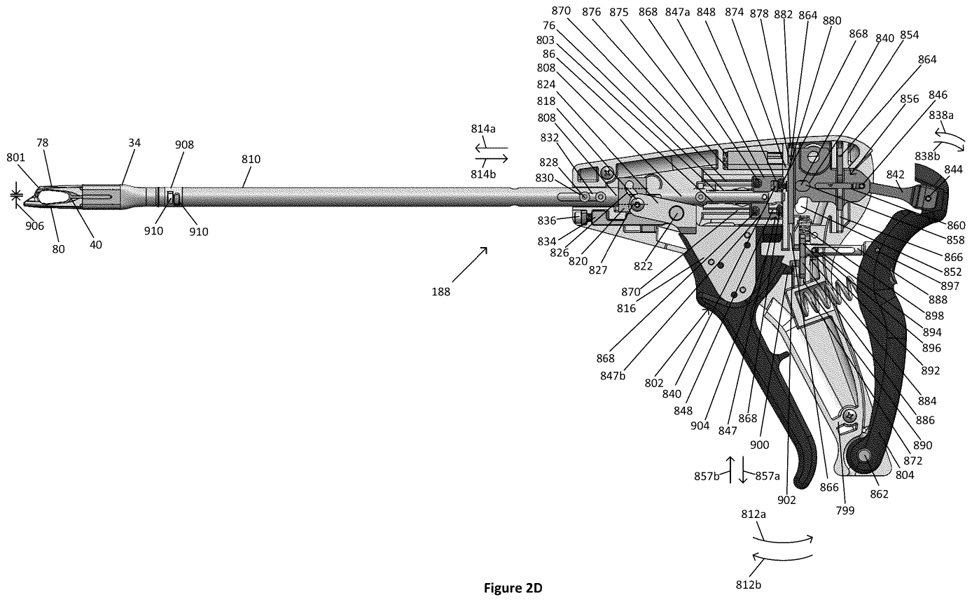

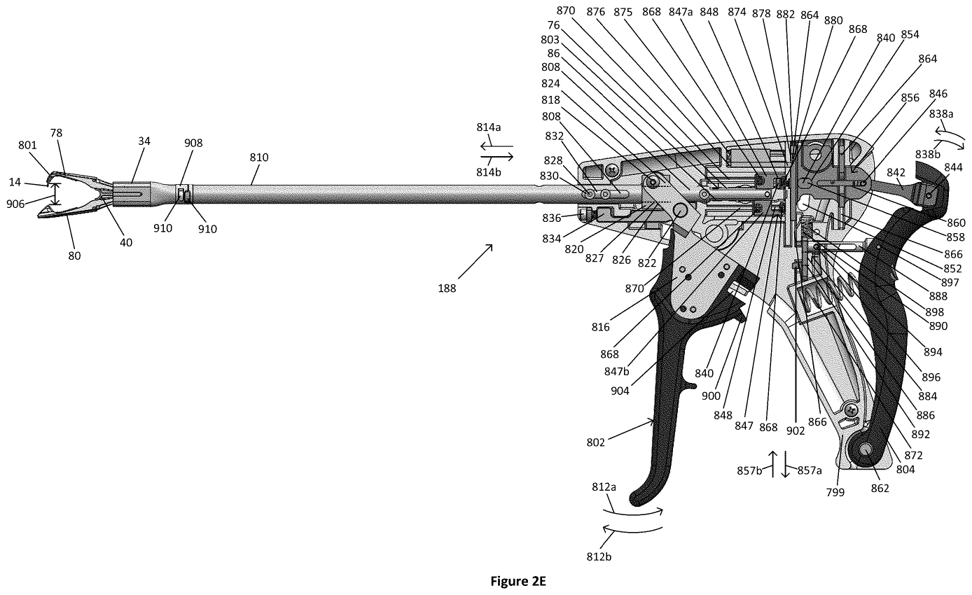

FIGS. 1-3E illustrate that the device 188 can have a suture 70. The suture 70 can be attached to the shuttle 14, for example, to a suture holder 18 of the shuttle 14. The suture 70 can be moved (e.g., pushed) back and forth through tissue 74 (e.g., see FIGS. 2A, 2B, 3A, 3B, and 31A) to suture the tissue 74, for example, by moving (e.g., pushing) the shuttle 14 back and forth between the jaws 801. When the shuttle 14 is moved from the first jaw 78 to the second jaw 80, the suture 70 can follow (e.g., can be pulled by) the shuttle 14 through the tissue 74. When the shuttle 14 is moved from the first jaw 78 to the second jaw 80, the suture 70 can follow (e.g., can be pulled by) the shuttle 14 from the first jaw 78 to the second jaw 80. When the shuttle 14 is moved from the second jaw 80 to the first jaw 78, the suture 70 can follow (e.g., can be pulled by) the shuttle 14 through the tissue 74. When the shuttle 14 is moved from the second jaw 80 to the first jaw 78, the suture 70 can follow (e.g., can be pulled by) the shuttle 14 from the second jaw 80 to the first jaw 78. The tissue 74 can be soft tissue. The tissue 74 can be hard tissue. The tissue 74 can include soft tissue and hard tissue. The tissue 74 can be, for example, a tendon, a ligament, a muscle, or any combination thereof. For example, the tissue 74 can be a rotator cuff. FIGS. 1-3E illustrate, for example, that the device 188 can be used to pass the suture 70 through the tissue 74 with or without removing the device 188 or the suture 70 from the target site while creating one or more complete stitches, where each complete stitch can be, for example, one pass of the suture 70 through the tissue 74 by moving the shuttle 14 from the first jaw 78 to the second jaw 80 or vice versa. The device 188 can make multiple passes of the suture 70 through the tissue 74 with or without extracting or reloading the suture passing device 188.

FIGS. 1-3E illustrate that the device 188 can have an inner tube 808, an outer tube 810, and a handle 104. The jaws 801 can be the distal end of the device 188 and the handle 104 can be the proximal end of the device 188 or vice versa. The inner tube 808 can be inside the outer tube 810. The inner tube 808 can connect the jaws 801 to the handle 104. For example, a proximal end of the inner tube 808 can be attached to the handle 104 and a distal end of the inner tube 808 can be attached to the jaws 801. The outer tube 810 can be movable. The outer tube 810 can be movable relative to the handle 104. The outer tube 810 can be movable relative to the inner tube 808. The outer tube 810 can be movable relative to the pushers 803. The outer tube 810 can be movable relative to the jaws 801. The outer tube 810 can be movable relative to the shuttle 14. The inner tube 808 can have a lumen for the pushers 803. The pushers 803 can be movable in the inner tube 808, for example, in the lumen of the inner tube 808. For example, the pushers 803 can be translatable, slidable, pushable, and/or pullable in the inner tube 808. The inner tube 808 can be, for example, cylindrical. The outer tube 810 can have a lumen for the inner tube 808 and the pushers 803. The pushers 803 can be movable in the outer tube 810, for example, in the lumen of the outer tube 810. For example, the pushers 803 can be translatable, slidable, pushable, and/or pullable in the outer tube 810. The outer tube 810 can be, for example, cylindrical. FIGS. 1-3E illustrate that the inner tube 808 and the outer tube 810 can be straight. As another example, the inner tube 808 and the outer tube 810 can be curved. The inner tube 808 and the outer tube 810 can be rigid and/or flexible.

FIGS. 1-3E illustrate that the device 188 can have a closer 34 and an opener 40. The closer 34 can close the jaws 801. The opener 40 can open the jaws 801. The outer tube 808 can have the closer 34. The closer 34 can be a distal end of the outer tube 808. The closer 34 can be attached to or integrated with the distal end of outer tube 808. The outer tube 808 can have the opener 40. The opener 40 can be attached to or integrated with the distal end of outer tube 808. The opener 40 can be attached to or integrated with the closer 34 or vice versa.

FIGS. 1-3E illustrate that the closer 34 and the opener 40 can be movable to close and open the jaws 801, respectively. The closer 34 and the opener 40 can be, for example, movable relative to the jaws 801 or vice versa. The closer 34 and the opener 40 can be, for example, movable relative to the pushers 803. The closer 34 and the opener 40 can be, for example, movable relative to the handle 104. The closer 34 and the opener 40 can be movable, for example, with the outer tube 810. The closer 34 and the opener 40 can, for example, move longitudinally in unison with the outer tube 810. The closer 34 and the opener 40 can, for example, move rotationally in unison with the outer tube 810. The jaws 801 can be closed from an open configuration to a closed configuration, for example, by moving the closer 34 (e.g., via a control on the handle 104) distally over the jaws 801, for example, away from the handle 104. The closer 34 can be, for example, a cover that can compress or squeeze the jaws 801 closed. The jaws 801 can be opened from a closed configuration to an open configuration, for example, by moving the opener 40 (e.g., via a control on the handle 104) proximally between the jaws 801, for example, toward the handle 104. The opener 34 can be, for example, a movable protrusion between the jaws 801 that can spread the jaws 801 apart.

FIGS. 1-3E illustrate that the handle 104 can have a first trigger 802, a second trigger 804, and a selector 806. The first trigger 802 can open and close the jaws 801, the second trigger 804 can advance and retract the pushers 803, and the selector 806 can control which of the pushers 803 (e.g., the first pusher 76 or the second pusher 86) the second trigger 804 controls.

The first trigger 802 can open and close the jaws 801, for example, by advancing and retracting the closer 34 and the opener 40 relative to the jaws 801. As another example, the first trigger 802 can open and close the jaws 801, for example, by advancing and retracting the jaws 801 relative to the closer 34 and the opener 40.

The second trigger 804 can move the shuttle 14, for example, by advancing and retracting the pushers 803. The second trigger 804 can move the first pusher 76 and the second pusher 86 independently of each other. The second trigger 804 can move the pushers 803 one at a time. For example, when the second trigger 804 moves the first pusher 76, the second pusher 86 can remain stationary, for example, in a neutral position. The neutral position of the second pusher 86 can be, for example, a retracted position (e.g., a fully retracted position) of the second pusher 86. When the second trigger 804 moves the first pusher 76, the second pusher 86 can remain in a retracted position. For example, when the second trigger 804 advances and retracts the first pusher 76, the second pusher 86 can remain in a retracted position (e.g., in a fully retracted position). As another example, when the second trigger 804 moves the second pusher 86, the first pusher 76 can remain stationary, for example, in a neutral position. The neutral position of the first pusher 76 can be, for example, a retracted position (e.g., a fully retracted position) of the first pusher 76. When the second trigger 804 moves the second pusher 86, the first pusher 76 can remain in a retracted position. For example, when the second trigger 804 advances and retracts the second pusher 86, the first pusher 76 can remain in a retracted position (e.g., in a fully retracted position). As another example, the first and second pushers 76, 86 can be moved in concert with each other such that advancing the first pusher 76 retracts the second pusher 86, and such that advancing the second pusher 86 retracts the first pusher 76. For example, the first and second pushers 76, 86 can be connected (e.g., via a link, a pusher control, or both) such that the first and second pushers 76, 86 can be moved in concert with each other.

The selector 806 can select which of the pushers 803 (e.g., the first pusher 76 or the second pusher 86) the second trigger 804 controls, for example, by moving a mover 840 into and out of engagement with an engager 847 that can be attached to each of the pushers 803.

The first trigger 802, the second trigger 804, and the selector 806 can have the relative positions shown, for example, in FIGS. 1-3E. For example, the first trigger 802 can be a front trigger, the second trigger 804 can be a rear trigger, and the selector 806 can be between the first and second triggers 802, 804. The first trigger 802 can be a finger trigger. For example, the first trigger 802 can be moved (e.g., pulled and/or pushed) with one or more fingers. The second trigger 804 can be a palm trigger. For example, the second trigger 804 can be moved (e.g., pushed and/or pushed) with a palm. The selector 806 can be moved with one or more fingers (e.g., a thumb and/or an index finger). A selector 806 can be on one or both sides of the handle 104. For example, FIGS. 1-3E illustrate that the device 188 can have a selector 806 on a first side (e.g., left side) of the handle 104 and can have a selector 806 on a second side (e.g., right side) of the handle 104, for example, to accommodate both right-handed and left-handed users.

The first trigger 802, the second trigger 804, and the selector 806 can be any type of movable actuator, for example, a lever, a button, a knob, a switch, or any combination thereof.

FIGS. 1-3E illustrate that the handle 104 can be symmetrical about a device longitudinal axis 956. As another example, the handle 104 may not be symmetrical about the device longitudinal axis 956.

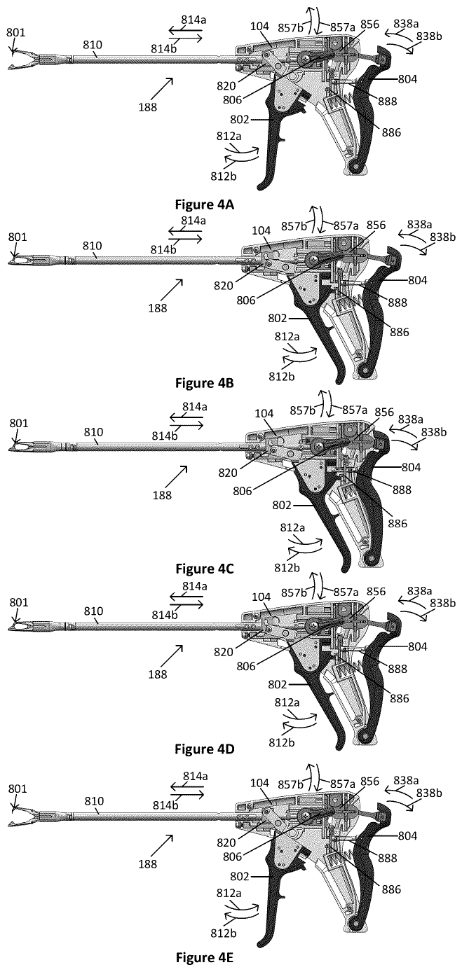

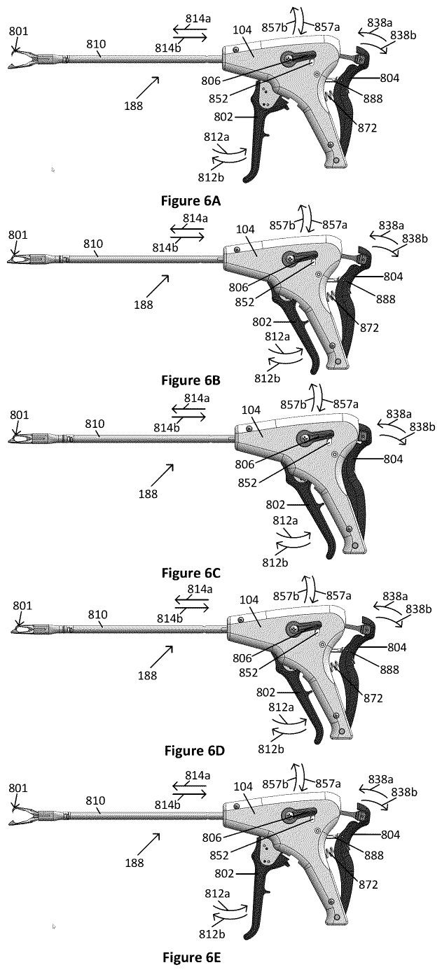

FIGS. 2A-2E illustrate an exemplary process of moving the shuttle 14 from the first jaw 78 to the second jaw 80. The process can include, for example, selecting the first pusher 76 with the selector 806 (e.g., as shown in FIG. 2A), closing the jaws 801 with the first trigger 802 (e.g., as shown in FIG. 2B), advancing the first pusher 76 with the second trigger 804 to move the shuttle 14 from the first jaw 78 to the second jaw 80 (e.g., as shown in FIG. 2C), retracting the first pusher 76 with the second trigger 804 (e.g., as shown in FIG. 2D), and opening the jaws 801 with the first trigger 802 (e.g., as shown in FIG. 2E). These steps or any sub-combination of these steps can be performed in any order, for example, in the order described in the previous sentence. The selector 806 and half (e.g., a left lateral half) of the handle 104 is shown transparent in FIGS. 2A-2E for illustrative purposes so that the components inside the handle 104 and their relationship and arrangement with each other can be seen. The position of the selector 806 in FIGS. 2A-2E is shown in FIGS. 4A-4E with half (e.g., the left lateral half) of handle 104 shown transparent and is shown in FIGS. 6A-6E.

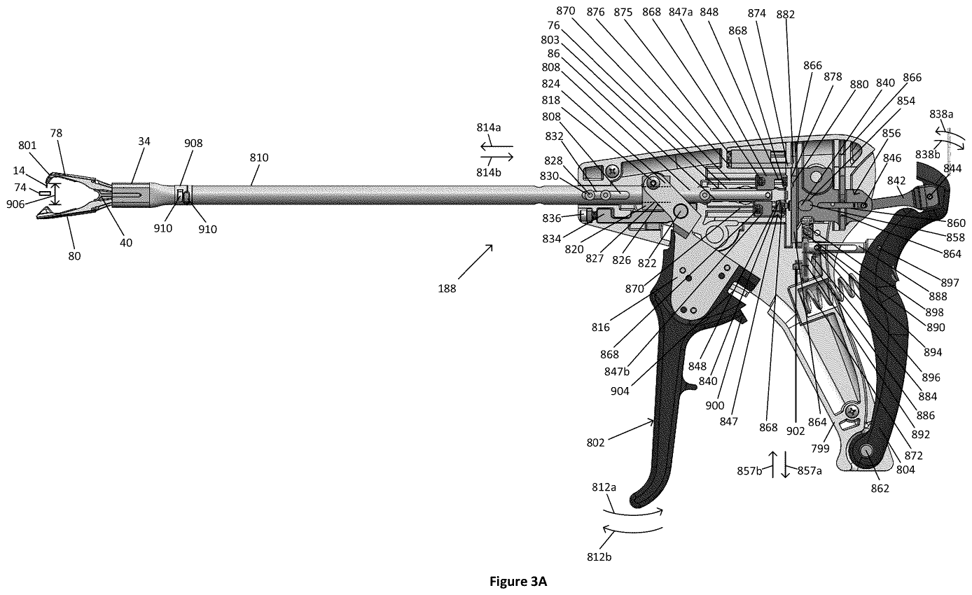

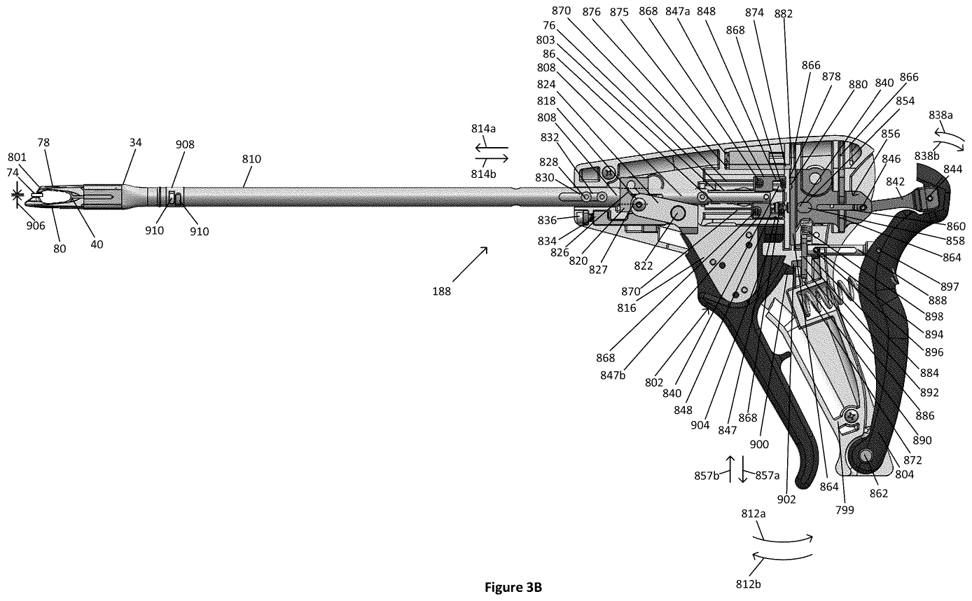

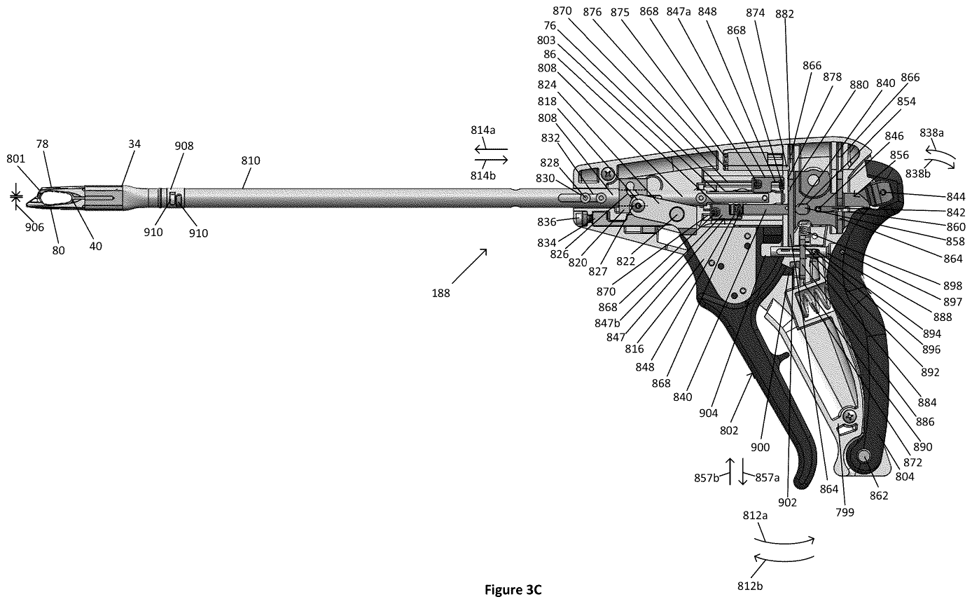

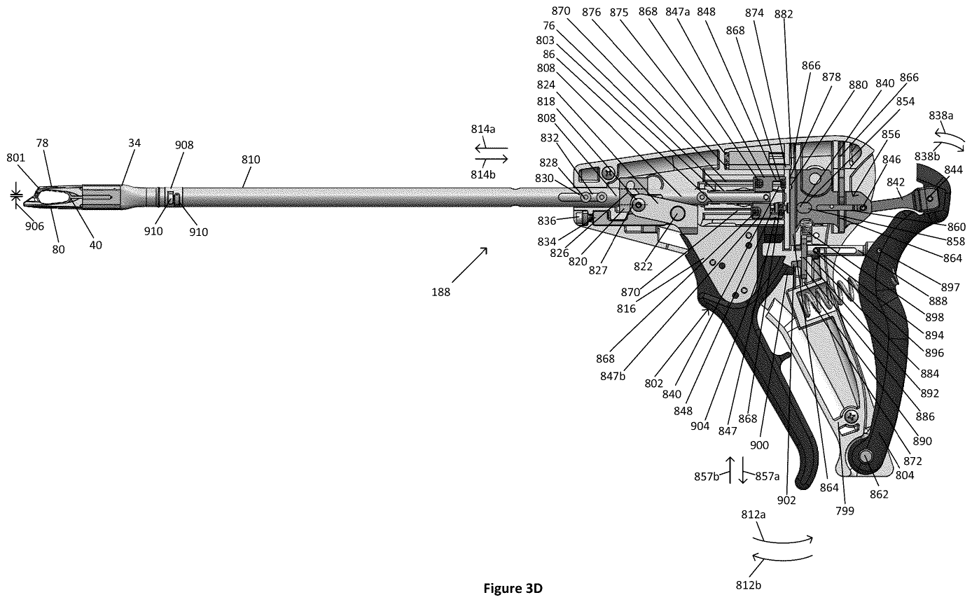

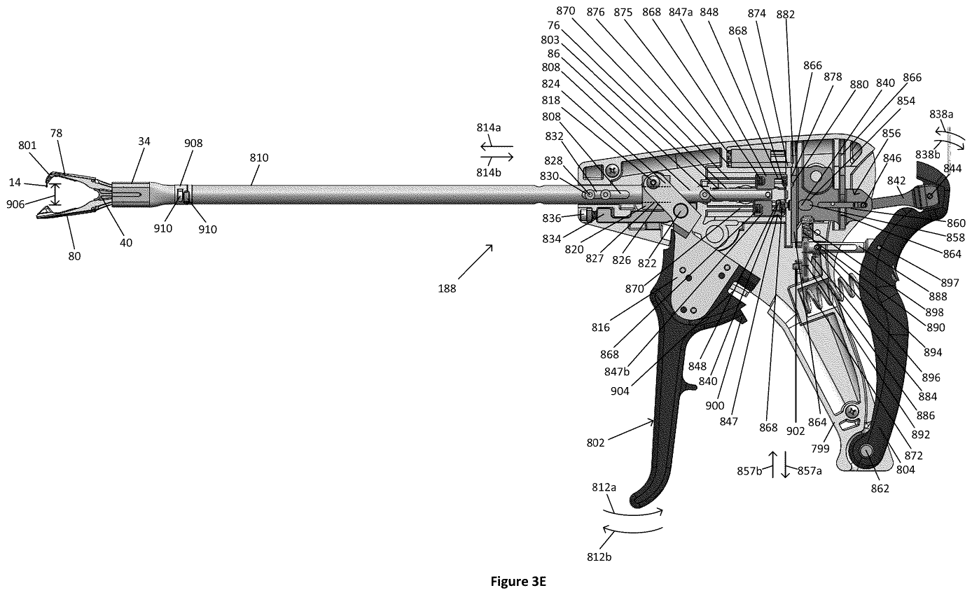

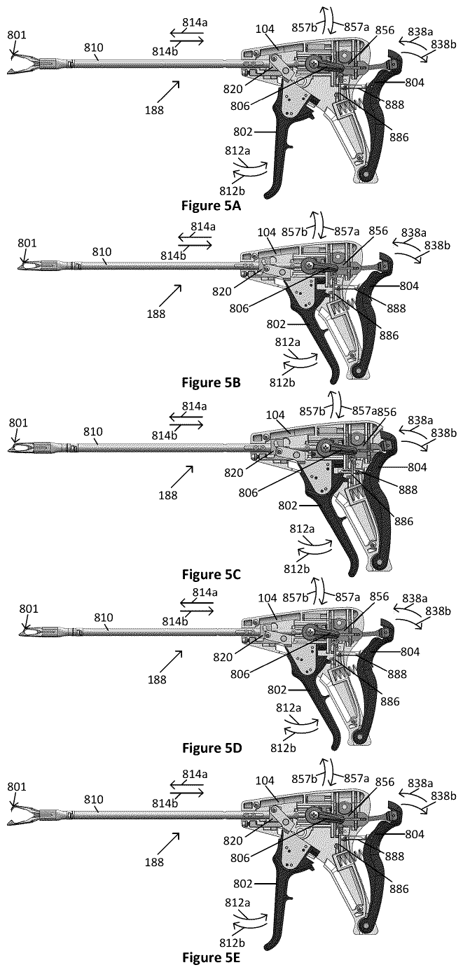

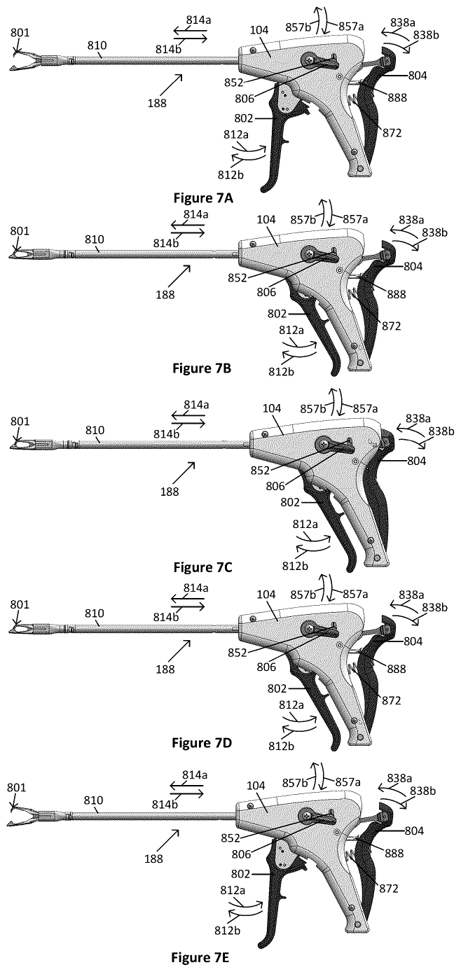

FIGS. 3A-3E illustrate an exemplary process of moving the shuttle 14 from the second jaw 80 to the first jaw 78. The process can include, for example, selecting the second pusher 86 with the selector 806 (e.g., as shown in FIG. 3A), closing the jaws 801 with the first trigger 802 (e.g., as shown in FIG. 3B), advancing the second pusher 86 with the second trigger 804 to move the shuttle 14 from the second jaw 80 to the first jaw 78 (e.g., as shown in FIG. 3C), retracting the second pusher 86 with the second trigger 804 (e.g., as shown in FIG. 3D), and opening the jaws 801 with the first trigger 802 (e.g., as shown in FIG. 3E). These steps or any sub-combination of these steps can be performed in any order, for example, in the order described in the previous sentence. The selector 806 and half (e.g., a left lateral half) of the handle 104 is shown transparent in FIGS. 3A-3E for illustrative purposes so that the components inside the handle 104 and their relationship and arrangement with each other can be seen. The position of the selector 806 in FIGS. 3A-3E is shown in FIGS. 5A-5E with half (e.g., a lateral half) of handle 104 shown transparent and is shown in FIGS. 7A-7E.

FIGS. 2A-3E illustrate that the first trigger 802 can be movable in a first direction 812a and a second direction 812b. The first direction 812a can be opposite the second direction 812b. The first trigger 802 can be moved in the first direction 812a, for example, by the user pulling (e.g., squeezing) the first trigger 802 toward a grip 799 of the handle 104. The first trigger 802 can be moved in the second direction 812b, for example, by the user pushing the first trigger 802 away from the grip 799. As another example, a spring (e.g., an auto-return spring) can move the first trigger 802 in the second direction 812b, for example, when the user lets go of the first trigger 802.

The jaws 801 can be closed by moving the first trigger 802 in the first direction 812a.

The jaws 801 can be opened by moving the first trigger 802 in the second direction 812b.

FIGS. 2A-2B and 3A-3B illustrate that the jaws 801 can be closed, for example, by moving the first trigger 802 in the first direction 812a from a first trigger first position to a first trigger second position. The first trigger first position can be the position of the first trigger 802 in FIGS. 2A and 3A. The first trigger second position can be the position of the first trigger 802 in FIGS. 2B and 3B. When the first trigger 802 is moved in the first direction 812a, the outer tube 810 can move in direction 814a which can move (e.g., advance) the closer 34 over the top of the jaws 801 in direction 814a to leverage the jaws 801 closed. When the closer 34 is moved over the top of the jaws 801 in direction 814a to leverage the jaws 801 closed, the closer 34 can push and/or slide against an outward facing surface of the first jaw 78 and can push and/or slide against an outward facing surface of the second jaw 80. This can cause the jaws 801 to close, for example, by causing the tips of the jaws 801 to rotate toward each other. The outward facing surfaces that the closer 34 can be engageable with can face away from the device longitudinal axis 956.

FIGS. 2D-2E and 3D-3E illustrate that the jaws 801 can be opened, for example, by moving the first trigger 802 in the second direction 812b from the first trigger second position to the first trigger first position. The first trigger first position can be the position of the first trigger 802 in FIGS. 2E and 3E. The first trigger second position can be the position of the first trigger 802 in FIGS. 2D and 3D. When the first trigger 802 is moved in the second direction 812b, the outer tube 810 can move in direction 814b which can move (e.g., retract) the opener 40 between the jaws 801 in direction 814b to leverage the jaws 801 open. When the opener 40 is moved between the jaws 801 in direction 814b to move the jaws 801 apart, the opener 40 can push and/or slide against an inward facing surface (e.g., a ramp) of the first jaw 78 and can push and/or slide against an inward facing surface (e.g., a ramp) of the second jaw 80. This can cause the jaws 801 to open, for example, by causing the tips of the jaws 801 to rotate away from each other. The inward facing surfaces that the opener 40 can be engageable with can face toward the device longitudinal axis 956.

FIGS. 2A-3E illustrate that the first trigger 802 can be connected to the outer tube 810, the closer 34, and/or the opener 40. FIGS. 2A-3E illustrate that the first trigger 802 can move the outer tube 810, the closer 34, and/or the opener 40. For example, the first trigger 802 can move the outer tube 810, the closer 34, and/or the opener 40 with a mover 820. The mover 820 can be movable, for example, via the first trigger 802. The mover 820 can move in directions 814a and 814b. The mover 820 can move the outer tube 810 in directions 814a and 814b. Direction 814a can be opposite direction 814b. Directions 814a and 814b can be parallel to a longitudinal axis of the device 188, for example, parallel to a longitudinal axis of the outer tube 810, or, as another example, parallel to the device longitudinal axis 956. The mover 820 can push and pull the outer tube 810. For example, the mover 820 can push the outer tube 810 in direction 814a and the mover 820 can pull the outer tube 810 in direction 814b. When the first trigger 802 is moved in the first direction 812a, the mover 820 can move in direction 814a, which can move the outer tube 810 in direction 814a. This can cause the closer 34 to move over the top of the jaws 801 in direction 814a to leverage the jaws 801 closed. When the first trigger 802 is moved in the second direction 812b, the mover 820 can move in direction 814b, which can move the outer tube 810 in direction 814b. This can cause the opener 40 to move proximally between the jaws 801 in direction 814b to leverage the jaws 801 open.

FIGS. 2A-3E illustrate that the first trigger 802 can be connected to the outer tube 810, for example, via a connector 816, a connector 818, and the mover 820, or any combination thereof. The connector 816 can be a first connector. The connector 818 can be a second connector. The mover 820 can be a third connector. For example, the first trigger 802 can be attached to the connector 816, the connector 816 can be attached to the connector 818, the connector 818 can be attached to the mover 820, and the mover 820 can be attached to the outer tube 810. A proximal end of the outer tube 810 can be, for example, welded to the mover 820.

The outer tube 810 can terminate at the mover 820. For example, a proximal terminal end of the outer tube 810 can be inside the mover 820 or can be attached to an outer surface of the mover 820.

The connector 816 can be movable. The connector 816 can be movable by the first trigger 802. The connector 816 can be rotatable about a pin 822. The connector 816 can pivot about the pin 822 when the first trigger 802 is moved in direction 812a (e.g., when the first trigger 802 is pulled) and when the first trigger 802 is moved in direction 812b (e.g., when the first trigger 802 is pushed). The first trigger 802 and the connector 816 can rotate together about the pin 822. The connector 816 can have arms 824, for example, one arm 824 on each side of the mover 820.

The connector 818 can be, for example, an extension that extends from the connector 816. The device 188 can have, for example, one or two connectors 818 (e.g., a first connector 818 and/or a second connector 818), such that a connector 818 can extend from each arm 824 of the connector 816. The connectors 818 (e.g., two connectors 818) can be on opposite sides of the mover 820. Each connector 818 can extend into the mover 820. A first portion of the connectors 818 (e.g., a first longitudinal end) can be outside the mover 820 and a second portion of the connectors 818 (e.g., a second longitudinal end) can be inside the mover 820. The connectors 818 can be attached to or integrated with the connector 816. For example, each of the connectors 818 can be attached to or integrated with one of the arms 824. Each connector 818 can be, for example, a screw or a pin that can extend through one of the arms 824 and into the mover 820. The connectors 818 can be movable. The connectors 818 can be movable in the mover 820. Movement of the connectors 818 in the mover 820 can cause the mover 820 to move in directions 814a and 814b. The connectors 818 can be movable by the first trigger 802.

The mover 820 can be, for example, a plug. The mover 820 can be, for example, a follower plug. The mover 820 can be, for example, a block. The mover 820 can be for example, a connector. The first trigger 802 can be linked to the outer tube 810 via the mover 820. The mover 820 can be, for example, an engager. For example, the mover 820 can engage with the outer tube 810 to move the closer 34 and/or the opener 40.

The mover 820 can have a channel 826 on one or two sides of the mover 820. For example, FIGS. 2A-3E illustrate that the mover 820 can have a channel 826 on two opposite sides (e.g., two lateral sides) of the mover 820 such that the mover 820 can have two channels 826, for example, a first channel 826 and a second channel 826. Each of the connectors 818 can be movable in one of the channels 826. For example, an end of one of the connectors 818 can be movable in one of the channels 826, and an end of another one of the connectors 818 can be movable in another one of the channels 826. The connectors 818 can extend into the channels 826. The channels 826 can be, for example, a groove or a slot in the mover 820. The channels 826 can be tracks for the connectors 818. For example, each connector 818 can ride in one of the channels 826 when the first trigger 802 is moved in directions 812a and 812b (e.g., when the first trigger 802 is moved back and forth). The connectors 818 can slide in the channels 826 as the first trigger 802 is moved in the first direction 812a. The connectors 818 can slide in the channels 826 as the first trigger 802 is moved in the second direction 812b. The mover 820 can have a channel 826, for example, on one side of the mover 820. As another example, the mover 820 can have a channel 826 on two sides (e.g., two opposite sides) of the mover 820. For example, FIGS. 2A-3E illustrate that the mover 820 can have a channel 826 on opposite lateral sides of the mover 820. In such variations, the device 188 can have two channels 826 and two connectors 818, for example, such that one of the two connectors 818 can ride in one of the two channels 826 and such that the other of the two connectors 818 can ride in the other of the two channels 826. As the first trigger 802 is moved back and forth, for example, in directions 812a and 812b, the outer tube 810 and the mover 820 can move in unison. The connectors 818 can be cams and the channels 826 can be cam follower paths.

FIGS. 2A-2B and FIGS. 3A-3B illustrate, for example, that the connectors 818 can ride from a first end of the channels 826 to a second end of the channels 826 as the first trigger 802 is moved in the first direction 812a from the first trigger first position shown in FIGS. 2A and 3A to the first trigger second position shown in FIGS. 2B and 3B. As the first trigger 802 is moved in the first direction 812a, the connectors 818 can push against a distal surface of the mover 820 defining the channels 826. This can cause the mover 820 to move (e.g., push) the outer tube 810 in direction 814a such that more of the outer tube 810 is outside the handle 104 when the jaws 801 are in a closed configuration (e.g., the closed configuration in FIGS. 2B and 3B) than when the jaws 801 are in an open configuration (e.g., the open configuration in FIGS. 2A and 3A).

FIGS. 2D-2E and FIGS. 3D-3E illustrate that the connectors 818 can ride from the second end of the channels 826 to the first end of the channels 826 as the first trigger 802 is moved in second direction 812b from the first trigger second position shown in FIGS. 2D and 3D to the first trigger first position shown in FIGS. 2E and 3E (e.g., manually and/or via an auto-return spring). As the first trigger 802 moves or is moved in the second direction 812b, the connectors 818 can push against a proximal surface of the mover 820 defining the channels 826. This can cause the mover 820 to move (e.g., pull) the outer tube 810 in direction 814b such that more of the outer tube 810 is inside the handle 104 when the jaws 801 are in an open configuration (e.g., the open configuration in FIGS. 2E and 3E) than when the jaws 801 are in a closed configuration (e.g., the closed configuration in FIGS. 2D and 3D).

The open configuration of the jaws 801 in FIGS. 2A and 2E can be an open configuration of the jaws 801. The open configuration of the jaws 801 in FIGS. 2A and 2E can be a fully open configuration of the jaws 801. The closed configuration of the jaws 801 in FIGS. 2B-2D can be a closed configuration of the jaws 801. The closed configuration of the jaws 801 in FIGS. 2B-2D can be a fully closed configuration of the jaws 801. A partially open configuration of the jaws 801 can be any open configuration of the jaws 801 between the fully closed configuration of the jaws 801 in FIGS. 2B-2D and the fully open configuration of the jaws 801 in FIGS. 2A and 2E. A partially closed configuration of the jaws 801 can be any closed configuration of the jaws 801 between the fully closed configuration of the jaws 801 in FIGS. 2B-2D and the fully open configuration of the jaws 801 in FIGS. 2A and 2E.

The inner tube 808 can be inside (e.g., can extend through) the mover 820. For example, the mover 820 can have a channel 827 that the inner tube 808 can extend through. The channel 827 can be, for example, a longitudinal channel that extends through the mover 820 from a first longitudinal end to a second longitudinal end. The channel 827 can be a central longitudinal channel through the mover 820. The outer tube 810 and the mover 820 can be movable (e.g., longitudinally translatable) over the inner tube 808.

FIGS. 2A-3E illustrate that the inner tube 808 can be attached to the handle 104, for example, via pins 828 and connectors 830. The pins 828 and the connectors 830 can mount the inner tube 808 to the handle 104. The connectors 830 can be, for example, bushings that step up the size of the pins 828 for connection to the handle 104. The connectors 830 can be hypotubes. The pins 828 can extend through connectors 830. Some of the pins 828 can extend through the outer tube 810. For example, the outer tube 810 can have a channel 832 on each side so that the pins 828 (e.g., two of the pins 828) can extend through the outer tube 810 and connect the inner tube 808 to the handle 104. The outer tube 810 can ride over the pins 828 that are in the channels 832. The ends of the pins 828 can attach the inner tube 808 to the handle 104.

The pins 828 can keep the pushers 803 spread apart and can support the pushers 803, for example, in the inner tube 808. The pins 828 can separate the pushers 803 from each other in the inner tube 808. The pins 828 can inhibit or prevent the pushers 803 from buckling, for example, when the pushers 803 are advanced by the second trigger 804. The pins 828 can limit and/or inhibit bending, kinking, and buckling of the pushers 803, or any combination thereof. For example, FIGS. 3A-3E illustrate that the central portion of the pins 828 can support the pushers 803 by limiting and/or inhibiting bending, kinking, or buckling of the pushers 803 in the inner tube 808 as the pushers 803 are advanced by the second trigger 804. The central portion of the pins 828 can support the pushers 803 by limiting the extent that the pushers 803 can bend, kink, or buckle in the inner tube 808 as the pushers 803 are advanced by the second trigger 804. The central portion of the pins 828 can support the pushers 803 by inhibiting the pushers 803 from bending, kinking, or buckling in the inner tube 808 as the pushers 803 are advanced by the second trigger 804. A pin 828 can be placed across the inner tube 808 between the pushers 803 at an interval of, for example, every 0.5 inches to every 3.0 inches, including every 0.5 inch increment within this range.

FIGS. 2A-3E illustrate that the first trigger 802 can rotate about the pin 822.

FIGS. 2A-3E illustrate that the handle 104 can have a trigger adjust yoke 834 and a trigger adjust screw 836. Tightening or loosening the trigger adjust screw 836 can adjust (e.g., longitudinally adjust) the position of the pin 822. This can move the outer tube 810 in directions 814a and 814b as desired. Adjusting the trigger adjust screw 836 can, for example, adjust the gap (e.g., the gap 906) between the jaws 801 when the jaws 801 are in a fully closed configuration. The gap (e.g., the gap 906) can be the zero-clamp size of the jaws 801 when the jaws 801 are closed. The gap can be adjusted as desired, for example, to account for tolerances in the trigger assemblies and/or to adjust the closing force of the jaws 801 by moving the trigger adjust yoke 834 in directions 814a and 814b by tightening and loosening the trigger adjust screw 836.

FIGS. 2A-3E illustrate that the second trigger 804 can be movable in a first direction 838a and a second direction 838b. The first direction 838a can be opposite the second direction 838b. The second trigger 804 can be moved in the first direction 838a, for example, by the user pushing (e.g., squeezing) the second trigger 804 toward the grip 799. The second trigger 804 can be moved in the second direction 838b, for example, by a spring 872. The spring 872 can be, for example, a coil spring. The spring 872 can be, for example, a compression spring. The spring 872 can bias the second trigger 804 in a non-actuated position (e.g., the position of the second trigger 804 shown in FIGS. 2A and 3A). The spring 872 can be an auto-return spring that can move the second trigger 804 in the second direction 838b. For example, when the second trigger 804 is released from a pushed (e.g., squeezed) position, the second trigger 804 can be pushed away from the grip 799 (e.g., pushed backwards) via the spring 872 such that the second trigger 804 can automatically return to a neutral position when the user releases the second trigger 804. As another example, the second trigger 804 can be manually moved in the second direction 838b by the user pushing or pulling the second trigger 804 away from the grip 799.

The second trigger 804 can move the pushers 803 independently of each other. The pushers 803 can be advanced, for example, by moving the second trigger 804 in the first direction 838a. The pushers 803 can be retracted, for example, by moving the second trigger 804 in the second direction 838b.

FIGS. 2B and 2C illustrate that the first pusher 76 can be advanced, for example, by moving the second trigger 804 in the first direction 838a from a second trigger first position to a second trigger second position. The second trigger first position can be the position of the second trigger 804 in FIG. 2B and the second trigger second position can be the position of the second trigger 804 in FIG. 2C. When the second trigger 804 is moved in the first direction 838a, the second trigger 804 can advance (e.g., push) the first pusher 76 in direction 814a to move the shuttle 14 from the first jaw 78 to the second jaw 80.

FIGS. 2C and 2D illustrate that the first pusher 76 can be retracted, for example, by moving the second trigger 804 in the second direction 838b from the second trigger second position to the second trigger first position. The second trigger second position can be the position of the second trigger 804 in FIG. 2C and the second trigger first position can be the position of the second trigger 804 in FIG. 2D. When the second trigger 804 is moved in the second direction 838b, the second trigger 804 can retract (e.g., pull) the first pusher 76 in direction 814b.

FIGS. 2B-2Dillustrate that when the second trigger 804 moves the first pusher 76, the second pusher 86 can remain in a retracted position. For example, when the second trigger 804 advances and retracts the first pusher 76, the second pusher 86 can remain in a retracted position (e.g., in a fully retracted position).

FIGS. 3B and 3C illustrate that the second pusher 86 can be advanced, for example, by moving the second trigger 804 in the first direction 838a from a second trigger first position to a second trigger second position. The second trigger first position can be the position of the second trigger 804 in FIG. 3B and the second trigger second position can be the position of the second trigger 804 in FIG. 3C. When the second trigger 804 is moved in the first direction 838a, the second trigger 804 can advance (e.g., push) the second pusher 86 in direction 814a to move the shuttle 14 from the second jaw 80 to the first jaw 78.

FIGS. 3C and 3D illustrate that the second pusher 86 can be retracted, for example, by moving the second trigger 804 in the second direction 838b from the second trigger second position to the second trigger first position. The second trigger second position can be the position of the second trigger 804 in FIG. 3C and the second trigger first position can be the position of the second trigger 804 in FIG. 3D. When the second trigger 804 is moved in the second direction 838b, the second trigger 804 can retract (e.g., pull) the second pusher 86 in direction 814b.

FIGS. 3B-3D illustrate that when the second trigger 804 moves the second pusher 86, the first pusher 76 can remain in a retracted position. For example, when the second trigger 804 advances and retracts the second pusher 86, the first pusher 76 can remain in a retracted position (e.g., in a fully retracted position).

FIGS. 2C and 2D and FIGS. 3C and 3D illustrate that the first pusher 76 and the second pusher 86 can be retracted, for example, by moving the second trigger 804 in the second direction 838b from the second trigger second position to the second trigger first position. The user and/or the spring 872 can move the spring 872 in direction 838b. For example, the spring 872 can bias the second trigger 804 to be in the second trigger first position. The spring 872 can be an auto-return spring such that the first pusher 76 can be retracted by releasing the second trigger 804. When the user releases (e.g., lets go) of the second trigger 804, the spring 872 can return (e.g., push) the second trigger 804 to the second trigger first position. The second trigger 804 can be released gradually such that the user can control the rate at which the first pusher 76 is retracted, for example, by applying a force against the second trigger 804 in direction 838a that is less than the force applied by the spring 872 in direction 838b. The second trigger 804 can be released all at once (e.g., the user can completely let go of the second trigger 804) such that the spring 872 can return the second trigger 804 in direction 838b to the second trigger first position without any resistance from the user in direction 838a. The user can pull the second trigger 804 in direction 838b.

FIGS. 2A-3E illustrate that the second trigger 804 can move the pushers 803 with a mover 840. The mover 840 can move the pushers 803 in directions 814a and 814b. The mover 840 can push and pull the pushers 803. For example, the mover 840 can push the pushers 803 in direction 814a and the mover 840 can pull the pushers 803 in direction 814b. The pushers 803 can be moved in directions 814a and 814b by moving the mover 840 in directions 814a and 814b. The mover 840 can be moved in direction 814a, for example, by moving the second trigger 804 in direction 838a (e.g., by pressing the second trigger 804). The mover 840 can be moved in direction 814b, for example, by moving the second trigger 804 in direction 838b (e.g., by releasing the second trigger 804). When the second trigger 804 is pressed and released, the second trigger 804 can rotate about a pin 862. The mover 840 can be, for example, a bar. The mover 840 can be, for example, a rod. The mover 840 can be, for example, a ram. The mover 840 can be, for example, a ram bar. The mover 840 can ram the pushers 803 in directions 814a and 814b. The mover 840 can be, for example, an engager. The mover 840 can, for example, releasably engage with the first pusher 76. The mover 840 can, for example, releasably engage with the second pusher 86.

FIGS. 2A-3E illustrate that the second trigger 804 can be connected to the mover 840, for example, via a connector 842. The second trigger 804 can be linked to the mover 840 via the connector 842. The connector 842 can be, for example, a bar. The connector 842 can be, for example, a rod. The connector 842 can be, for example, a connecting rod. A first end of the connector 842 can be attached to the second trigger 804 and a second end of the connector 842 can be attached to the mover 840. The connector 842 can rotate relative to the second trigger 804. For example, the first end of the connector 842 can be attached to the second trigger 804 with a pin 844 such that the first end of the connector 842 can pivot about the pin 844. The connector 840 can rotate relative to the mover 840. For example, the second end of the connector 842 can be attached to the mover 840 with a pin 846 such that the second end of the connector 842 can pivot about the pin 846.

FIGS. 2A-3E illustrate that the selector 806 can move the mover 840 with a mover 856. The mover 856 can move the mover 840 in directions 857a and 857b. Direction 857a can be opposite direction 857b. Direction 857a can be, for example, downwards (e.g., toward a base of the grip 799) and direction 857b can be, for example, upwards (e.g., away from the base of the grip 799). Directions 857a and 857b can be at an angle (e.g., a perpendicular angle) to directions 814a and 814b. The mover 856 can push and/or pull the mover 840. For example, the mover 856 can push or pull the mover 840 in direction 857a and the mover 856 can push or pull the mover 840 in direction 857b. The mover 856 can be, for example, a connector. The mover 856 can be, for example, a plate. The mover 856 can be, for example, a switcher plate. The mover 840 can be moved in direction 857a by moving the mover 856 in direction 857b. The mover 856 can be moved in direction 857a, for example, by moving the selector 806 in direction 857a. The mover 840 can be moved in direction 857b by moving the mover 856 in direction 857b. The mover 856 can be moved in direction 857b, for example, by moving the selector 806 in direction 857b.

The handle 104 can have one or two selectors 806 such that a selector 806 can be on one or both sides of the handle 104. For example, FIGS. 2A-3E illustrate that the device 188 can have two selectors 806, one on each side (e.g., each lateral side) of the handle 104. The two selectors 806 can be opposite each other. The two selectors 806 can be linked via the mover 856 such that moving one of the two selectors 806 can move both of the two selectors 806. As another example, the two selectors 806 can be independently movable. An extension 850 (e.g., see FIG. 12) can extend from each of the selectors 806, through an opening 852 in a side of the handle 104, and into an opening 854 in the mover 856. The selector 806 can be linked to the mover 856 via the extension 850. The opening 852 can be a slot in the handle 104, for example, a curved slot in the handle 104. The opening 854 can be a recess or a through-hole in the mover 856. For example, FIGS. 2A-3E illustrate that the opening 854 can be a through-hole.

FIGS. 2A-3E illustrate that the mover 856 can have a channel 858 (e.g., which can be seen through the opening 852 in FIGS. 2A-3E). The channel 858 can be, for example, a longitudinal channel that extends through the mover 856 from a first longitudinal end to a second longitudinal end. The channel 858 can be a central longitudinal channel through the mover 856.

The channel 858 can have the position shown in FIGS. 2A-3E. The mover 840 can be movable in the channel 858, for example, along a longitudinal axis of the channel 858. The mover 840 can be movable in the channel 858, for example, in directions 814a and 814b. For example, the mover 840 can be advanced and retracted in the channel 858, for example, by moving the second trigger 804 in directions 838a and 838b (e.g., by pressing and releasing the second trigger 804). The connector 842 can be movable in the channel 858, for example, along the longitudinal axis of the channel 858. The connector 842 can be movable in the channel 858, for example, in directions 814a and 814b. For example, the connector 842 can be advanced and retracted in the channel 858, for example, by moving the second trigger 804 in directions 838a and 838b (e.g., by pressing and releasing the second trigger 804). The channel 858 can be a track for the mover 840. The channel 858 can guide the mover 840 as the second trigger 804 is moved in directions 838a and 838b. The mover 856 can, for example, restrict relative movement of the mover 840 along axes perpendicular to the longitudinal axis of the channel 858. For example, the surface or surfaces (e.g., wall or walls) of the mover 856 that define the channel 858 can prevent the mover 840 from moving relative to the mover 856 along axes perpendicular to the longitudinal axis of the channel 858.

The mover 856 can have a channel 860 on one or two sides of the mover 856. For example, FIGS. 2A-3E illustrate that the mover 856 can have a channel 860 on two opposite sides (e.g., two lateral sides) of the mover 856 such that the mover 856 can have two channels 860. The pin 846 can be movable in the channels 860. The pin 846 can extend into the channels 860.

The channels 860 can be, for example, a groove or a slot. FIGS. 2A-3E illustrate, for example, that the channels 860 can be a slot. The channels 860 can be tracks that the pin 846 (e.g., the ends of the pin 846) can ride in when the second trigger 804 is moved in directions 838a and 838b (e.g., when the second trigger 804 is pressed and released). The pin 846 can slide in the channels 860 in direction 814a as the second trigger 804 is moved in the first direction 838a. The pin 846 can slide in the channels 860 in direction 814b as the second trigger 804 is moved in the second direction 838b. The slots 860 can guide the pin 846 such that movement of the pin 846 in the slots 860 can be restricted to linear movement, for example, parallel to the longitudinal axis of the outer tube 810. The pin 846 and the slots 860 can thereby convert the rotational motion of the second trigger 804 into translational motion of the mover 840.

FIGS. 2A-3E illustrate that the mover 856 can have protrusions 864. The protrusions 864 can be, for example, fins. The protrusions 864 can ride in grooves 866 in the handle 104. The grooves 866 can be grooves in the wall of the of the handle 104. The protrusions 864 can be slidable in the grooves 866. The grooves 866 can be tracks that the protrusions 864 can slide in when the mover 856 is moved by the selector 806. The grooves 866 can provide travel guidance (e.g., linear travel guidance) for the mover 856. For example, the protrusions 864 and the grooves 866 can permit transverse movement of the mover 856 (e.g., directions 857a and 857b in FIGS. 2A-3E). For example, the extension 850 can move (e.g., push) the mover 856 in direction 857a when the selector 806 is moved in direction 857a and the extension 850 can move (e.g., push) the mover 856 in direction 857b when the selector 806 is moved in direction 857b. The protrusions 864 and the grooves 866 can restrict or prevent longitudinal movement of the mover 856 (e.g., directions 814a and 814b in FIGS. 2A-3E). The protrusions 864 and the grooves 866 can restrict or prevent lateral movement of the mover 856 (e.g., into and out of the page in FIGS. 2A-3E).

FIGS. 2A-3E illustrate that the proximal end of each of the pushers 803 can be connected to an engager 847. For example, the proximal end of the first pusher 76 can be attached to a first engager 847a and the proximal end of the second pusher 86 can be attached to a second engager 847b. The first engager 847a and the second engager 847b (also referred to collectively as the engagers 847) can be, for example, a block. The engagers 847, can be for example, a connector. The engagers 847 can each be, for example, a ram catch. The engagers 847 can, for example, catch the mover 840. The mover 840 can be releasably engageable with the engagers 847. The mover 840 can move the pushers 803 in direction 814a, for example, by moving the engagers 847 in direction 814a. The mover 840 can move the pushers 803 in direction 814b, for example, by moving the engagers 847 in direction 814b. The mover 840 can push and pull the engagers 847. For example, the mover 840 can push the engagers 847 in direction 814a and the mover 840 can pull the engagers 847 in direction 814b. The mover 840 can move the first and second engagers 847a, 847b independently of each other.

FIGS. 2A-3E illustrate that each of the engagers 847 can have a groove 848. The mover 840 can be movable into and out of the grooves 848, for example, via the selector 806. The mover 840 can be moved into and out of the grooves 848, for example, via the selector 806. The distal end of the mover 840 can be movable into and out of the grooves 848, for example, via the selector 806. The distal end of the mover 840 can be moved into and out of the grooves 848, for example, via the selector 806.

FIGS. 2A-3E illustrate, for example, that the selector 806 can move (e.g., push) the mover 840 in direction 857a into the groove 848 of the second engager 847b when the selector 806 is moved in direction 857a. FIGS. 2A-3E illustrate, for example, that the selector 806 can move (e.g., push) the mover 840 in direction 857b into the groove 848 of the first engager 847a when the selector 806 is moved in direction 857b. When the device 188 is upright, direction 857a can be downwards and direction 857b can be upwards. For example, the selector 806 can be moved from a selector first position (e.g., the up position shown in FIGS. 2A-2E, 4A-4E, and 6A-6E) to a selector second position (e.g., the down position shown in FIGS. 3A-3E, 5A-5E, and 7A-7E) to move the mover 840 in direction 857a out of the groove 848 of the first engager 847a and into the groove 848 of the second engager 847b to select the second pusher 86. As another example, the selector 806 can be moved from a selector second position (e.g., the down position shown in FIGS. 3A-3E, 5A-5E, and 7A-7E) to a selector first position (e.g., the up position shown in FIGS. 2A-2E, 4A-4E, and 6A-6E) to move the mover 840 in direction 857b out of the groove 848 of the second engager 847b and into the groove 848 of the first engager 847a to select the first pusher 76. The selector first and second positions can be top and bottom positions of the selector 806, respectively, such that selector positions can match the position of the shuttle 14. For example, the selector first position (e.g., up position) can be selected when the shuttle 14 is in the first jaw 78 (e.g., the upper jaw) and the user would like to move the shuttle from the first jaw 78 to the second jaw 80. As another example, the selector second position (e.g., down position) can be selected when the shuttle 14 is in the second jaw 80 (e.g., the lower jaw) and the user would like to move the shuttle from the second jaw 80 to the first jaw 78. Such an arrangement can give the user an intuitive feel for the device 188 and can let the user know at a glance which one of the pushers 803 is selected.

FIGS. 2A-3E illustrate that the mover 840 can be releasably engageable with the first engager 847a. For example, when the mover 840 is in the groove 848 of the first engager 847a, the mover 840 can be releasably engaged with the first engager 847a. When the mover 840 is in the groove 848 of the first engager 847a, the mover 840 can advance the first pusher 76 by advancing (e.g., pushing) the first engager 847a in direction 814a and the mover 840 can retract the first pusher 76 by retracting (e.g., pulling) the first engager 847a in direction 814b.

FIGS. 2A-3E illustrate that the mover 840 can be releasably engageable with the second engager 847b. For example, when the mover 840 is in the groove 848 of the second engager 847b, the mover 840 can be releasably engaged with the second engager 847b. When the mover 840 is in the groove 848 of the second engager 847b, the mover 840 can advance the second pusher 86 by advancing (e.g., pushing) the second engager 847b in direction 814a and the mover 840 can retract the second pusher 86 by retracting (e.g., pulling) the second engager 847b in direction 814b.

FIGS. 2A-3E illustrate that the selector 806 can change the position of the mover 840 by changing the position of the mover 856 such that the mover 840 can follow the mover 856 in the channel 858 as the mover 856 is moved by the selector 806. The mover 856 can move (e.g., push) the mover 840 as the mover 856 is moved by the selector 806. The selector 806 can thereby select which of the pushers 803 to engage with by aligning the mover 840 with the first engager 847a or by aligning the mover 840 with the second engager 847b. The mover 840 can be aligned with the first engager 847a, for example, by moving the mover 840 into the groove 848 in the first engager 847a. The mover 840 can be aligned with the second engager 847b, for example, by moving the mover 840 into the groove 848 in the second engager 847b. FIGS. 2A-2E illustrate exemplary configurations of the device 188 in which the mover 840 is aligned with the first engager 847a. FIGS. 3A-3E illustrate exemplary configurations of the device 188 in which the mover 840 is aligned with the second engager 847b.

The selector 806 can have two selectable positions, for example, a selector first position (e.g., a top position, an upper position) and a selector second position (e.g., a bottom position, a lower position). The selector 806 can be moved from the selector first position to the selector second position and from the selector second position to the selector first position. When the selector 806 is in the selector first position, the first pusher 76 can be selected by virtue of the mover 840 being aligned with the first engager 847a. When the selector 806 is in the selector first position, the mover 840 can be aligned with the first engager 847a such that the first pusher 76 can be advanced and retracted by pushing and pulling the first engager 847a with the mover 840 via the second trigger 804. When the selector 806 is in the selector second position, the second pusher 86 can be selected by virtue of the mover 840 being aligned with the second engager 847b. When the selector 806 is in the selector second position, the mover 840 can be aligned with the second engager 847b such that the second pusher 86 can be advanced and retracted by pushing and pulling the second engager 847b with the mover 840 via the second trigger 804.

When the user would like to move the shuttle 14 from the first jaw 78 to the second jaw 80, the selector 806 can be moved to the selector first position, for example, by moving the selector in direction 857b. When the user would like to move the shuttle 14 from the second jaw 80 to the first jaw 78, the selector 806 can be moved to the selector second position, for example, by moving the selector 806 in direction 857a. The mover 840 and the mover 856 can have the positions shown in FIGS. 2A-2E when the selector 806 is in the selector first position. The selector first position can be the position of the selector 806 shown in FIGS. 4A-4E. The mover 840 and the mover 856 can have the positions shown in FIGS. 3A-3E when the selector 806 is in the selector second position. The selector second position can be the position of the selector 806 shown in FIGS. 5A-5E. When the selector 806 is moved from the selector first position to the selector second position (e.g., in direction 857a), the extension 850 can move (e.g., push) the mover 856 in direction 857a. When the selector 806 is moved from the selector second position to the selector first position, the extension 850 can move (e.g., push) the mover 856 in direction 857b.

FIGS. 2A-3E illustrate that a detent 874 can provide tactile and/or audible feedback to the user when the selector 806 reaches the selector first position (e.g., top position) and when the selector 806 reaches the selector second position (e.g., bottom position). The detent 874 can be, for example, a slider. The detent 874 can be, for example, a bullnose slider, for example, having the shape shown in FIGS. 2A-3E. The detent 874 can be movable (e.g., slidable). For example, the detent 874 can be movable (e.g., slidable) in directions 814a and 814b, for example, in grooves 876 in the handle 104.

The detent 874 can interface with a surface of the mover 856. For example, FIGS. 2A-3E illustrate that the detent 874 can interface with the mover 856, for example, with a surface 878 of the mover 856. The surface 878 of the mover 856 can be a face of the mover 856. The surface 878 of the mover 856 can be, for example, a distal face of the mover 856. The mover 856 can move relative to the detent 874 such that the detent 874 can slide or drag along the surface 878 as the selector 806 is moved from the selector first position to the selector second position and as the selector 806 is moved from the selector second position the selector first position. The detent 874 can be loaded with a spring 875. The spring 875 can be, for example, a coil spring. The spring 875 can be, for example, a compression spring. The spring 875 can be, for example, a bullnose slider spring. The spring 875 can have the position illustrated, for example, in FIGS. 2A-3E. When the detent 874 is in contact with the surface 878 of the mover 856, the spring 875 can be compressed.

When the selector 806 reaches the selector first position, the detent 874 can releasably engage with the with the mover 856. For example, FIGS. 2A-2E illustrate that when the selector 806 is in the selector first position, the detent 874 can be releasably positioned in a recess 880 on the surface 878 of the mover 856. The recess 880 can be, for example, a groove or a hole. When the detent 874 reaches the recess 880, the spring 875 can move (e.g., snap) the detent 874 into the recess 880 in direction 814b. This can provide the user with a tactile click and/or an audible click to let the user know that the selector first position (e.g., top position) is selected. The spring 875 can move (e.g., snap) the detent 874 into the recess 880 in direction 814b by expanding. When the detent 874 is in the recess 880, the mover 840 can be aligned with the first pusher 76.

When the selector 806 reaches the selector second position, the detent 874 can releasably engage with the with the mover 856. For example, FIGS. 3A-3E illustrate that when the selector 806 is in the selector second position, the detent 874 can be releasably positioned in a space 882 adjacent the mover 856. The space 882 can be, for example, adjacent a face (e.g., the top face) and/or an edge (e.g., the top distal edge) of the mover 856. When the detent 874 reaches the space 882, the spring 875 can move (e.g., snap) the detent 874 into the space 882 (e.g., above the top face and/or top distal edge). This can provide the user with a tactile click and/or an audible click to let the user know that the selector second position (e.g., bottom position) is selected. The spring 875 can move (e.g., snap) the detent 874 into the space 882 by expanding. When the detent 874 is in the space 882, the mover 840 can be aligned with the second pusher 86.

As another example, the surface 878 can have two recesses 880, one recess 880 to indicate that the selector first position has been reached and one recess 880 to indicate that the selector second position has been reached.

FIGS. 2A-3E illustrate, for example, that the detent 874 can engage with a groove on the surface 878 of the mover 856 when the selector 806 is in the selector first position, and can engage with the top of the mover 856 when the selector 806 is in the selector second position.

The detent 874 and the recess 880 can hold the mover 856 and the selector 806 in the positions shown in FIGS. 2A-2E. For example, FIGS. 2A-2E illustrate that when the detent 874 is releasably positioned in the recess 880, the mover 856 can be held in a first position by the detent 874 and the recess 880. FIGS. 2A-2E thereby illustrate that the detent 874 can hold the selector 806 in the selector first position and can give the user feedback (e.g., audible and/or tactile feedback) when the selector 806 reaches the selector first position to let the user know that the second trigger 804 can be actuated (e.g., pushed) to advance the first pusher 76. The detent 874 and the recess 880 can let the user know when they have fully selected the first pusher 76 by indicating to the user that the selector 806 is in the selector first position (e.g., the top position).

The selector 806 can be in the selector first position, for example, when the mover 840 is fully positioned in the groove 848 of the first engager 847a such that the first pusher 76 can be advanced by the second trigger 804. For example, the user can hear and/or feel a click when the mover 856 becomes aligned with the first pusher 76.

The detent 874 and the space 882 can hold the mover 856 and the selector 806 in the positions shown in FIGS. 3A-3E. For example, FIGS. 3A-3E illustrate that when the detent 874 is releasably positioned in the space 882, the mover 856 can be held in a second position by the detent 874 and the space 882. FIGS. 3A-3E thereby illustrate that the detent 874 can hold the selector 806 in the selector second position and can give the user feedback (e.g., audible and/or tactile feedback) when the selector 806 reaches the selector second position to let the user know that the second trigger 804 can be actuated (e.g., pushed) to advance the second pusher 86. The detent 874 and the space 882 can let the user know when they have fully selected the second pusher 86 by indicating to the user that the selector 806 is in the selector second position (e.g., the bottom position). The selector 806 can be in the selector second position, for example, when the mover 840 is fully positioned in the groove 848 of the second engager 847b such that the second pusher 86 can be advanced by the second trigger 804. For example, the user can hear and/or feel a click when the mover 856 becomes aligned with the second pusher 86.

FIGS. 2B and 2C illustrate that the second trigger 804 can advance the first pusher 76 by moving the bar 840 in direction 814a when the mover 840 is aligned with the first engager 847a.

FIGS. 2C and 2D illustrate that the second trigger 804 can retract the first pusher 76 by moving the bar 840 in direction 814b when the mover 840 is aligned with the first engager 847a.

FIGS. 3B and 3C illustrate that the second trigger 804 can advance the second pusher 86 by moving the bar 840 in direction 814a when the mover 840 is aligned with the second engager 847b.

FIGS. 3C and 3D illustrate that the second trigger 804 can retract the second pusher 86 by moving the bar 840 in direction 814b when the mover 840 is aligned with the second engager 847b.

FIGS. 2A-3E illustrate that the engagers 847 can have protrusions 868. The protrusions 868 can be, for example, lateral protrusions. The protrusions 868 can be, for example, bosses. The protrusions 868 can be, for example, fins. The protrusions 868 can ride in grooves 870 in the handle 104. The protrusions 868 can be slidable in the grooves 870. The grooves 870 can be tracks that the protrusions 868 can slide in when the engagers 847 are moved by the second trigger 804. The grooves 870 can provide travel guidance (e.g., linear travel guidance) for the engagers 847. For example, the protrusions 868 and the grooves 870 can permit longitudinal movement of the engagers 847 (e.g., in directions 814a and 814b). The protrusions 868 and the grooves 870 can restrict or prevent lateral movement of the engagers 847 (e.g., into and out of the page in FIGS. 2A-3E). The protrusions 868 and the grooves 870 can restrict or prevent transverse movement of the engagers 847 (e.g., in directions 857a and 857b).

The engagers 847 can have protrusions 868 on two sides (e.g., two opposite sides). For example, FIGS. 2A-3E illustrate that the engagers 847 can each have four protrusions 868, two on a first lateral side and two on a second lateral side. The first lateral side can be opposite the second lateral side. Two of the protrusions 868 can be distal the groove 848 and two of the protrusions 868 can be proximal the groove 848. The two protrusions distal the groove 848 can be opposite each other. The two protrusions proximal the groove 848 can be opposite each other. As another example, the protrusions 868 can be a fin that extends along the length of the engagers 847 (e.g., along the entire length or along 70% or more of the entire length), for example, along both lateral sides of the engagers 847. The fins can be opposite each other. Using bosses for the protrusions 868 as opposed to fins can reduce friction between the engagers 847 and the handle 104. The pushers 803 (e.g., rods of the pushers 803) can buckle a little when the pushers 803 are compressed as the engagers 847 are advanced using the second trigger 804, and the bosses can allow the engagers 847 to wiggle around a little bit when the engagers 847 move, for example, more than fins would allow, which can be helpful to the overall function and reliability of the device 188.

FIGS. 2A-3E illustrate that a lockout 884 can prevent the second trigger 804 from being actuated until the first trigger 802 is actuated (e.g., pulled). The lockout 884 can prevent the second trigger 804 from being actuated until the first trigger 802 is fully actuated (e.g., fully pulled back). For example, the lockout 884 can prevent the second trigger 804 from being moved in direction 838a until the first trigger 802 is in the first trigger second position. The lockout 884 can prevent the second trigger 804 from being actuated (e.g., moved in direction 838a) until the jaws 801 are closed. The lockout 884 can prevent the second trigger 804 from being actuated (e.g., moved in direction 838a) until the jaws 801 are fully closed.

FIGS. 2A-3E illustrate that the lockout 884 can have a lockout gate 886 and a lockout bar 888. The lockout gate 886 can have a closed position and an open position. The closed position can be a lockout position. The open position can be a non-lockout position. When the lockout gate 886 is in the lockout position, the lockout gate 886 can block the lockout bar 888 from being moved in direction 814a. When the lockout gate 886 is in the open position, the lockout gate 886 may not block the lockout bar 888 from being moved in direction 814a. For example, when the lockout gate 886 is in the open position, the lockout bar 888 can moved past the lockout gate 886 in direction 814a, for example, through an opening 892 in the lockout gate 886 in direction 814a. The lockout bar 888 can be aligned with the opening 892 when the jaws 801 are fully closed. In less than fully closed positions, lockout bar 888 can be misaligned with the opening 892 such that the lockout gate 886 can block the lockout bar 888.

FIGS. 2A-3E illustrate that the lockout gate 886 can be raised and lowered to open and close the lockout gate 886, respectively. For example, the lockout gate 886 can be moved in directions 857b and 857a to open and close the lockout gate 886, respectively. The lockout gate 886 can be raised and lowered to unblock and block the lockout bar 888, respectively. For example, the lockout gate 886 can be moved in directions 857b and 857a to unblock and block the lockout bar 888, respectively.

The lockout gate 886 can be opened by the first trigger 802. The lockout gate 886 can be moved from the closed position to the open position by the first trigger 802. For example, squeezing the first trigger 802 can move (e.g., lift) the lockout gate 886 to align the opening 892 of the lockout gate 886 with the lockout bar 888, and once the opening 892 is aligned with the lockout bar 888, the lockout bar 888 can be free to move forward in direction 814a such that the second trigger 804 can be actuated to advance one of the pushers 803. FIGS. 2A, 2E, 3A, and 3E illustrate an exemplary closed position of the lockout gate 886. FIGS. 2B-2Dand 3B-3D illustrate an exemplary open position of the lockout gate 886. The closed position of the lockout gate 886 can prevent the second trigger 804 from being actuated until the jaws 801 are fully closed (e.g., until the first trigger 802 is fully pulled back in direction 812a). When the jaws 801 are in less than a fully closed position, the lockout gate 886 can prevent the second trigger 804 from being actuated by blocking movement of the lockout bar 888 in direction 814a. The lockout gate 886 can thereby block the lockout bar 888 unless the jaws 801 are fully closed, for example, unless the first trigger 802 is fully squeezed.

FIGS. 2A-3E illustrate that the lockout gate 886 can be movable, for example, in directions 857a and 857b. The lockout gate 886 can slide in grooves 890 in the handle 104. The lockout gate 886 can be slidable in the grooves 890. The lockout gate 886 can be, for example, a plate. The lockout gate 886 can have the opening 892. The opening 892 can be, for example, a through-hole. The opening 892 can be, for example, a window in the lockout gate 886.

FIGS. 2A-3E illustrate that the lockout bar 888 can be movable, for example, in directions 814a and 814b. The lockout bar 888 can be movable into and movable out of the opening 892. The lockout bar 888 can be, for example, a rod. The lockout bar 888 can have a slot 894. The slot 894 can be a track for an extension 896. The extension 896 can be, for example, a rod or a pin in the handle 104. The extension 896 can extend through the slot 894. The lockout bar 888 can be moved relative to the extension 896 such that the extension 896 can provide travel guidance (e.g., linear travel guidance) for the lockout bar 888 when the lockout bar 888 is moved, for example, by the second trigger 804. The lockout bar 888 can be attached to the second trigger 804. For example, a first end (e.g., a proximal end) of the lockout bar 888 can be attached to the second trigger 804 with a pin 897 such that the lockout bar 888 can pivot about the pin 897. The extension 896 can be, for example, non-round which can allow for altering the starting position of the second trigger 804.

FIGS. 2A-3E illustrate that the lockout gate 886 can be movable by the first trigger 802 and by a lockout spring 898. For example, the lockout gate 886 can be moved in direction 857b by the first trigger 802, for example, by an extension 900 of the first trigger 802 when the first trigger 802 is moved in the first direction 812a. When the first trigger 802 is moved in the first direction 812a, the extension 900 can move (e.g., push) the lockout gate 886, for example, by pushing against an extension 902 of the lockout gate 886. The extension 900 can be a tab (also referred to as a lifting tab) that extends from the first trigger 802. The extension 902 can be a tab that extends from the lockout gate 886. The lockout gate 886 can be moved in in direction 857a by the first trigger 802 and/or by the lockout spring 898. For example, FIGS. 2A-3E illustrate that the lockout gate 886 can be moved in direction 857a by the lockout spring 898 when the first trigger 802 is moved in the second direction 812b. The lockout spring 898 can bias the lockout gate 886 in a lockout position (e.g., the position shown in FIGS. 2A, 2E, 3A, and 3E).

The lockout spring 898 can keep the lockout gate 886 in a lockout position regardless of the orientation of the device 188, for example, regardless of whether the device 188 is upright, tilted to one side, or upside down.

FIGS. 2A-3E illustrate that the lockout bar 888 can be movable by the second trigger 804.

FIGS. 2A-3E illustrate that the lockout 884 can have a closed configuration and an open configuration. The closed configuration can be a lockout configuration. The open configuration can be a non-lockout configuration. The closed configuration can be any configuration in which the lockout bar 888 is blocked by the lockout gate 886. The closed configuration can be any configuration in which the lockout bar 888 is not movable through the opening 892. The open configuration can be any configuration in which the lockout bar 888 is not blocked by the lockout gate 886. The open configuration can be any configuration in which the lockout bar 888 is movable past the lockout gate 886, for example, through the opening 892. For example, FIGS. 2A, 2E, 3A, and 3E illustrate an exemplary closed configuration of the lockout 884, and FIGS. 2B-2D and 3B-3D illustrate an exemplary open configuration of the lockout 884.

When the lockout 884 is in a closed configuration, the lockout bar 888 can be blocked by the lockout gate 886. For example, when the lockout 884 is in a closed configuration, the lockout bar 888 can be misaligned with the opening 892 such that the lockout gate 886 blocks the lockout bar 888 from moving, for example, in direction 814a. This misalignment can prevent the second trigger 804 from being actuated (e.g., from being moved in direction 838a). This misalignment can lock the second trigger 804 in a non-actuated position (e.g., in the second trigger first position).

When the lockout 884 is in an open configuration, the lockout bar 888 can be movable past the lockout gate 886, for example, through the opening 892. For example, when the lockout 884 is in an open configuration, the lockout bar 888 can be aligned with the opening 892 such that the lockout bar 888 can be moved into and out of the opening 892, for example, in directions 814a and 814b, for example, by moving the second trigger 804 in directions 838a and 838b, respectively. When the opening 892 is aligned with the lockout bar 888, the lockout spring 898 can be compressed. When the opening 892 is misaligned with the lockout bar 888, the lockout spring 898 can be less compressed than when the opening 892 is aligned with the lockout bar 888.

FIGS. 2A and 3A illustrate that when the lockout 884 is in a closed configuration, the lockout gate 886 can be in a lockout gate first position and FIGS. 2B-2Dand 3B-3D illustrate that when the lockout 884 is in an open configuration, the lockout gate 886 can be in a lockout gate second position. The lockout gate first position can be a dropped position of the lockout gate 886. The lockout gate first position can be a neutral position of the lockout gate 886. The lockout gate second position can be a raised position of the lockout gate 886. The lockout gate second position can be a lifted position of the lockout gate 886. The lockout gate 886 can be moved (e.g., raised) from the lockout gate first position to the lockout gate second position, for example, via the first trigger 802. The lockout gate 886 can be moved (e.g., lowered) from the lockout gate second position to the lockout gate first position, for example, via the lockout spring 898.

FIGS. 2A-3E illustrate that the lockout gate 886 can be movable in a first direction to open the lockout 884 and in a second direction to close the lockout 884. The first direction can be, for example, direction 857b. The second direction can be, for example, direction 857a. The lockout gate 886 can be moved in direction 857b to open the lockout 884, for example, from a closed configuration (e.g., the closed configuration shown in FIGS. 2A and 3A) to an open configuration (e.g., the open configuration shown in FIGS. 2B and 3B). FIGS. 2A-2B and FIGS. 3A-3B illustrate, for example, that moving the lockout gate 886 in direction 857b can align the opening 892 with the lockout bar 888. The lockout gate 886 can be moved in direction 857a to close the lockout 884, for example, from an open configuration (e.g., the open configuration shown in FIGS. 2D and 3D) to a closed configuration (e.g., the closed configuration shown in FIGS. 2E and 3E). FIGS. 2D-2E and FIGS. 3D-3E illustrate, for example, that moving the lockout gate 886 in direction 857a can misalign the opening 892 with the lockout bar 888.

FIGS. 2A-2B and 3A-3B illustrate that as first trigger 802 is moved in the first direction 812a, the first trigger 802 (e.g., via the extension 900) can move the lockout gate 886 in direction 857b to align the opening 892 with the lockout bar 888 so that the second trigger 804 can be moved in direction 838a to advance the pushers 803. FIGS. 2A-2B and FIGS. 3A-3B thereby illustrate that the second trigger 804 can be unlocked by aligning the opening 892 with the lockout bar 888, for example, by squeezing the first trigger 802.

FIGS. 2B-2C and 3B-3C illustrate that as the second trigger 804 is moved in the first direction 838a when the lockout 884 is in an open configuration, the lockout bar 888 can move through the opening 892 in direction 814a. FIGS. 2C and 3C illustrate that when the second trigger 804 is in the second trigger second position (e.g., when the first pusher 76 is fully advanced as shown in FIG. 2C and when the second pusher 86 is fully advanced as shown in FIG. 3C), the lockout bar 888 can be in the opening 892. FIGS. 2C and 3C illustrate that when the first and second triggers 802, 804 are both actuated, a distal end of the lockout bar 888 can be in a recess 904 in the first trigger 802. When the distal end of the lockout bar 888 is in the recess 904, the first trigger 802 can be locked in the first trigger second position, thereby locking the jaws 801 closed. In such variations, the first trigger 802 can be unlocked, for example, by moving the second trigger 804 in direction 838b, for example, by releasing the second trigger 804. As another example, when the distal end of the lockout bar 888 is in the recess 904, the first trigger 802 may not be locked in the first trigger second position such that the jaws 801 may not be locked closed.

FIGS. 2C-2D and 3C-3D illustrate that as the second trigger 804 moves in the second direction 838b when the lockout 884 is in an open configuration, the lockout bar 888 can move through the opening 892 in direction 814b.

FIGS. 2D-2E and 3D-3E illustrate that as first trigger 802 is moved in the second direction 812b, the lockout spring 898 can move the lockout gate 886 in direction 857a from the lockout gate second position to the lockout gate first position to misalign the opening 892 with the lockout bar 888 to releasably lock the second trigger 804 in the second trigger first position.

FIGS. 2A-3E illustrate that the lockout 884 can prevent the user from passing the shuttle 14 between the jaws 801 when the jaws 801 are open. FIGS. 2A-3E illustrate that the lockout 884 can prevent the user from passing the shuttle 14 between the jaws 801 when the jaws 801 are too far apart. This can be advantageous because if the shuttle 14 is pushed by the second trigger 804 when the jaws 801 are too far apart, the shuttle 14 may miss the opposite jaw, so the lockout 884 can prevent that by releasably locking the second trigger 804 in the second trigger first position until the jaws 801 are closed.

FIGS. 2A-3E illustrate, for example, that the lockout 884 can prevent the second trigger 804 from being actuated until the jaws 801 are fully closed. FIGS. 2A-3E illustrate, for example, that the lockout 884 can releasably lock second trigger 804 in the second trigger first position until the jaws 801 are fully closed. FIGS. 2A-3E illustrate, for example, that the lockout 884 can releasably lock second trigger 804 in the second trigger first position until the lockout bar 888 is movable through the opening 892. The lockout bar 888 can be movable through the opening 892 when the jaws 801 are fully closed. The jaws 801 can be considered fully closed (e.g., in a fully closed configuration) when the first trigger 802 is in the first trigger second position. The jaws 801 can be considered fully closed (e.g., in a fully closed configuration) when a gap 906 between the tip of the first jaw 78 and the tip of the second jaw 80 is between 0.00 mm and 1.50 mm, including every 0.01 mm increment within this range (e.g., 0.00 mm, 0.81 mm, 1.50 mm). The tip of the first jaw 78 can be, for example, a distal terminal end of the first jaw 78. The tip of the second jaw 80 can be, for example, a distal terminal end of the second jaw 80. The lockout 884 can open (e.g., unlock) when the gap 906 between the tips of the jaws 801 is within this range. For example, when the gap 906 is 1.50 mm or less, the lockout 884 can open (e.g., unlock). As another example, when the gap 906 is 1.00 mm or less, the lockout 884 can open (e.g., unlock). As another example, when the gap 906 is 0.8128 mm or less (0.032 inches or less), the lockout 884 can open (e.g., unlock). When the gap 906 is within one of these ranges (e.g., within an open range), the lockout bar 888 can be me moved into and out of the opening 892 to advance and retract the pushers 803.

FIGS. 2A-3E illustrate that the lockout 884 can be in a closed configuration when the jaws 801 are in an open configuration. For example, the lockout 884 can be in the closed configuration when the gap 906 is greater than 0.00 mm to 1.50 mm. For example, the lockout 884 can have the closed configuration when the gap 906 is greater than 1.50 mm, when the gap 906 is greater than 1.00 mm, when the gap 906 is greater than 0.8128 mm (0.032 inches), or when the gap 906 is greater than 0.00 mm. For example, FIGS. 2A and 3A illustrate an exemplary closed configuration of the lockout 884. The lockout 884 can be in the open configuration when the jaws 801 are in a closed configuration (e.g., a fully closed configuration). For example, the lockout 884 can be in the open configuration when the gap 906 is between 0.00 mm and 1.50 mm. For example, the lockout 884 can have the open configuration when the gap 906 is 1.50 mm or less, when the gap 906 is 1.00 mm or less, when the gap 906 is 0.8128 mm or less (0.032 inches or less), or when the gap 906 is 0.00 mm or less. When the lockout 884 is unlocked (e.g., when the lockout 884 is in the open configuration), the pushers 803 can be advanced and retracted with the second trigger 804. For example, FIGS. 2B and 3B illustrate an exemplary open configuration of the lockout 884.

When the jaws 801 clamp tissue, the gap 906 may not close to zero because there can be tissue between the tips of the jaws 801. The lockout 884 can help guarantee that the gap 906 is small enough so that the shuttle 14 can transit across the gap 906 to find the opposite jaw. The jaws 801 may or may not be closable to zero when there is no tissue between the jaws 801. The gap 906 can be less when the jaws 801 are fully closed without tissue between the jaws 801 than when the jaws 801 are fully closed with tissue between the jaws 801. This is because the tissue can inhibit or prevent the jaws 801 from being clamped to the smallest allowable gap 906. In such variations, the lockout 884 can help guarantee that the gap 906 is still small enough when the jaws 801 are fully closed against tissue so that the shuttle 14 can transit across the gap 906 to find the opposite jaw. As long as the gap 906 is within an open range, the lockout bar 888 can be me moved into and out of the opening 892 to advance and retract the pushers 803. The smallest allowable gap 906 can be the gap 906 between the jaws 801 when the jaws 801 are fully closed without tissue between the jaws 801. The smallest allowable gap 906 can be, for example, between 0.00 mm and 1.50 mm, including every 0.01 mm increment within this range (e.g., 0.00 mm, 0.81 mm, 1.50 mm).

FIGS. 2A-3E illustrate that the lockout gate 886 can prevent the second trigger 804 from being moved forward until the first trigger 802 is pulled back. When the lockout gate 886 is down (e.g., in the lockout gate first position) and the lockout bar 888 is proximal the lockout gate 886 (e.g., see FIGS. 2A and 3A), the lockout bar 888 can hit the lockout gate 886 when the second trigger 806 is moved in direction 838a so the second trigger 806 can be prevented from moving forward (e.g., in direction 838a). After the first trigger 802 lifts the lockout gate 886 (e.g., to the lockout gate second position), the lockout bar 888 can pass through the opening 892. The lockout gate 886 can thereby prevent the user from moving the shuttle 14 when the jaws 801 are not completely closed, for example, when the gap 906 is too large (e.g., greater than an open range for the gap 906). This can be advantageous because if the second trigger 804 is moved when the gap 906 between the jaws 801 is too large (e.g., larger than 0.00 mm to 1.50 mm), there can be a risk that the shuttle 14 can miss the opposite jaw when the shuttle is moved from the first jaw to the second jaw or vice versa. The lockout gate 886 can allow the shuttle 14 to be passed between the jaws 801 (e.g., can allow the second trigger 804 to be pushed) when the lockout bar 888 is aligned with the opening 892 when the jaws 801 are fully closed. In less than fully closed positions, the lockout gate 886 can block the second trigger 804. The second trigger 804 can thereby be blocked unless the jaws 801 are fully closed (e.g., unless the first trigger 802 is fully squeezed). Squeezing the first trigger 802 can lift the lockout gate 886 in direction 857b, and once lifted, the lockout bar 888 can be free to move through the opening 892 in direction 814a such that the second trigger 804 can be squeezed to advance the first pusher 76 or the second pusher 86.

FIGS. 2A-3E illustrate that the lockout gate 886 can be tunable such that the gap 906 can range from 0.00 mm to 1.50 mm between the jaws 801 (e.g., 0.8128 mm (0.032 inches)) when the jaws 801 are clamped onto tissue. For example, when the jaws 801 are closed and there is 0.8128 mm (0.032 inches) of tissue between the jaws 801 such that the gap 906 is 0.8128 mm (0.032 inches) between the jaws 801, it can take about a 45 lb closing force to close the jaws 801 to have the gap 906 of 0.8128 mm (0.032 inches). The lockout gate 886 can be tunable, for example, by adjusting the trigger adjust screw 836. As another example, the lockout gate 886 can be tunable by adjusting the position and/or length of the extension 900 on the first trigger 802, by adjusting the position and/or length of the extension 902 on the lockout gate 886, or by adjusting the positions and/or lengths of both. The extensions 900 and 902 can be screws. In such variations, the first trigger 802 and the lockout gate 886 can have threaded holes for the extensions 900 and 902, where the lengths of the extensions 900 and 902 can be selected to tune the lockout gate 886 as desired. As another example, the extensions 900 and 902 can be pre-fabricated tabs that can be trimmed to length. Tightening or loosening the trigger adjust screw 836 can move the first trigger 802 forward (e.g., distal) and backward (e.g., proximal), which can move the outer tube 810 relative to the jaws 801, which can move the closer 34 relative to the jaws 801, thereby adjusting the size of the gap 906 when the first trigger 802 is actuated.

FIGS. 2A-3E illustrate that the lockout bar 888 can set the travel limit. For example, the slot 894 can set the travel distance of the second trigger 804, the mover 840, the mover 856, and the lockout bar 888, or any combination thereof. The lockout bar 888 can thereby control the overall travel distance of various features and can lock and unlock the second trigger 804 with the lockout gate 886.

FIGS. 2A-3E illustrate that a first trigger assembly can include, for example, the first trigger 802, the connector 816, the connector 818, the mover 820, the outer tube 810, the closer 34, the opener 40, the lockout gate 886, or any combination thereof. The first trigger 802 can be, for example, an arm or a lever.

FIGS. 2A-3E illustrate that a second trigger assembly can include, for example, the second trigger 804, the connector 842, the mover 840, the engagers 847, the pushers 803, the lockout gate 886, the lockout bar 888, or any combination thereof. The second trigger 804 can be, for example, an arm or a lever.

FIGS. 2A-3E illustrate that a selector assembly can include, for example, the selector 806, the extension 850, the mover 856, or any combination thereof. The selector 806 can be, for example, an arm or a lever.

FIGS. 2A-3E illustrate that the closer 34 can be moved by the first trigger 802 in direction 814a to leverage the jaws 801 closed.

FIGS. 2A-3E illustrate that the opener 40 can be moved by the first trigger 802 in direction 814b to leverage the jaws open.

A first stitch can be created at a first stitch location with the suture 70, for example, using the process shown in FIGS. 2A-2E, and a second stitch can be created at a second stitch location with the suture 70, for example, using the process shown in FIGS. 3A-3E. For example, once the first stitch has been created, the user can then move to the next stitch location and move the selector 806 to the selector second position and then perform the steps shown in FIGS. 3A-3E to create the second stitch.

A first stitch can be created at a first stitch location with the suture 70, for example, using the process shown in FIGS. 3A-3E, and a second stitch can be created at a second stitch location with the suture 70, for example, using the process shown in FIGS. 2A-2E. For example, once the first stitch has been created, the user can then move to the next stitch location and move the selector 806 to the selector first position and then perform the steps shown in FIGS. 2A-2E to create the second stitch.

FIGS. 2A-2E illustrate an exemplary process of moving the shuttle 14 from the first jaw 78 to the second jaw 80, for example, to create a stitch with the suture 70. The process can include, for example, selecting the first pusher 76 with the selector 806, closing the jaws 801 with the first trigger 802, advancing the first pusher 76 with the second trigger 804 to move the shuttle 14 from the first jaw 78 to the second jaw 80, retracting the first pusher 76 with the second trigger 804, and opening the jaws 801 with the first trigger 802. These steps or any sub-combination of these steps can be performed in any order, for example, in the order described in the previous sentence. Selecting the first pusher 76 with the selector 806 can include moving the selector 806 to the selector first position or confirming that that the selector 806 is already in the selector first position.

FIGS. 3A-3E illustrate an exemplary process of moving the shuttle 14 from the second jaw 80 to the first jaw 78, for example, to create a stitch with the suture 70. The process can include, for example, selecting the second pusher 86 with the selector 806, closing the jaws 801 with the first trigger 802, advancing the second pusher 86 with the second trigger 804 to move the shuttle 14 from the second jaw 80 to the first jaw 78, retracting the second pusher 86 with the second trigger 804, and opening the jaws 801 with the first trigger 802. These steps or any sub-combination of these steps can be performed in any order, for example, in the order described in the previous sentence. Selecting the second pusher 86 with the selector 806 can include moving the selector 806 to the selector second position or confirming that that the selector 806 is already in the selector second position.

FIGS. 2A-3E illustrate that the device 188 can have suture holders 908. The suture holders 908 can manage the position the suture 70, for example, by holding the suture 70, by allowing the suture 70 to move through the suture holders 908 during stitching, or both. For example, the device 188 can have 1 to 5 suture holders 908, including every 1 suture holder increment within this range (e.g., 1 suture holder, 2 suture holders, 5 suture holders). FIGS. 2A-3E illustrate, for example, that the device 188 can have one suture holder 908. The suture holders 908 can be, for example, clips. The suture holders 908 can be, for example, sleeves.

The suture holders 908 can be fixed to the outer tube 810. The suture holders 908 can be in a fixed position, for example, welded to the outer tube 810. The suture holders 908 can be removably attached to the outer tube 810. For example, the suture holders 908 can be removably attached to the outer tube 810 (e.g., shaft) via a clip fit. The suture holders 908 can be snapped into place over the outer tube 810. The suture holders 908 can be adjustable on the outer tube 810. For example, the position of the suture holders 908 can be adjusted by moving the suture holders 908 relative to the outer tube 810. The suture holders 908 can be translatable (e.g., slidable) along the outer tube 810. The suture holders 908 can be rotatable about the outer tube 810. The suture holders 908 can be translatable and/or rotatable relative to the outer tube 810.

FIGS. 2A-3E illustrate that the suture holders 908 can have arms 910. The arms 910 can be, for example, cleats. The arms 910 can be, for example, bars. The suture 70 can be attached to the arms 910. As the shuttle 14 is moved back and forth between the jaws 801, the suture 70 can be pulled through the arms 910, for example, through the space that can be between the arms 910 and the outer tube 810. FIGS. 2A-3E illustrate, for example, that the suture holders 908 can have four arms 910, two arms 910 each on opposite sides of the suture holder 908.

Having arms 910 on both sides can advantageously allow the user to suture tissue from left to right (e.g., use the two arms 910 shown in FIGS. 2A-3E) or from right to left (e.g., use the two arms 910 opposite the two arms 910 shown in FIGS. 2A-3E. Having arms 910 on both sides can allow right-handed and left-handed users to both use the device 188 with ease. As another example, the arms 910 can be on only one side of the suture holders 908.

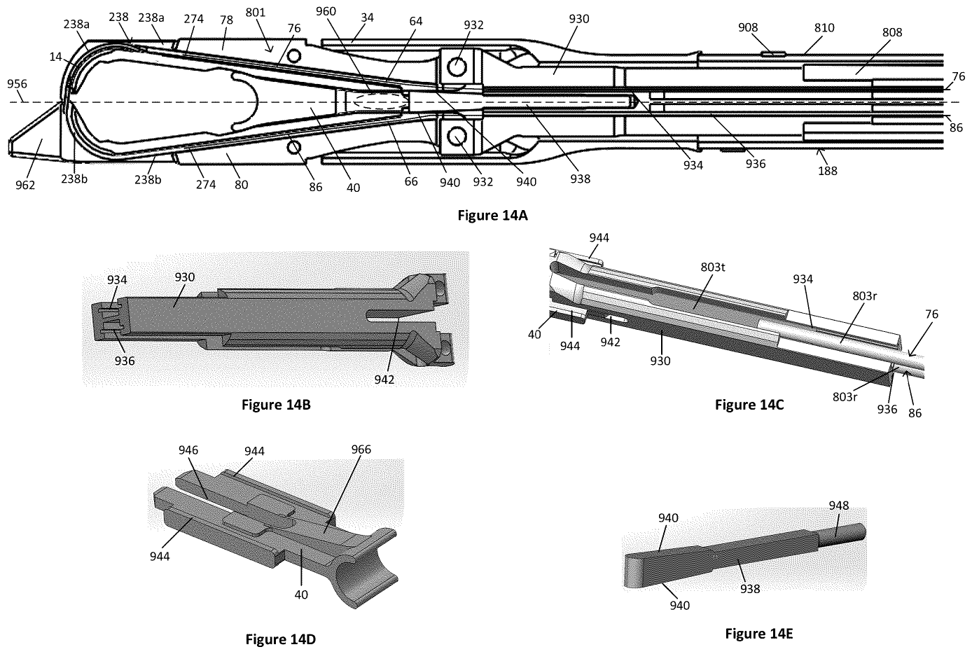

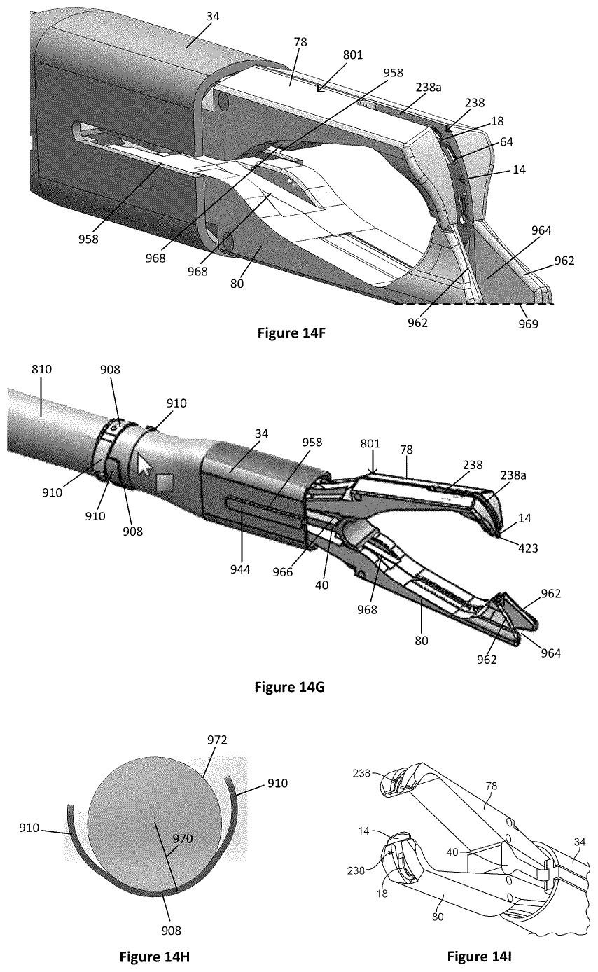

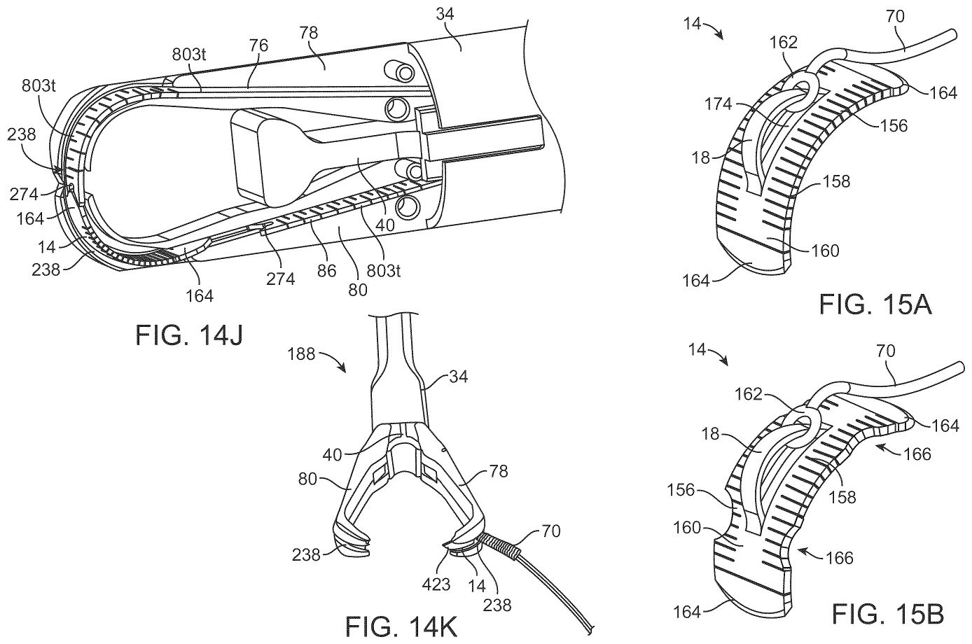

The suture holders 908 can manage the position of the suture 70. For example, the suture holders 908 can be positioned such that the suture holders 908 can hold the suture 70 to the side of the jaws 801 (e.g., instead of over the top of the jaws 801, where, for a frame of reference, the slots 238 in FIGS. 14A, 14F, and 14G on the outer surface of the jaws 801 can be considered over the top of the jaws 801). The suture holders 908 can allow the user to manage the suture 70 as the user goes from stitch to stitch so that the jaws 801 do not bite on the suture 70 and so that the suture 70 does not come over the top of the jaws 801. The suture holders 908 can position the suture 70 such that the biting the suture 70 with the jaws 801 can be prevented and so that the suture 70 can be prevented from coming over the top of the jaws 801. If the suture 70 is allowed to come over the top of the jaws 801, as the jaws 801 are opened, the suture 70 can be pulled tight, which the user can prevent with the suture holders 908. The suture holders 908 can keep the suture 70 off to the side so that the user does not have to worry about the suture 70 getting in the way when the jaws 801 are opened and closed. The suture holders 908 can guide the suture 70 back to the handle 104, for example, so that that suture can run along the side of the opener 40, down along the length of the outer tube 810, and out through the same cannula that the device 188 was introduced through.

The jaws 801 can have zero, one, or more (e.g., two) tissue guides 962. The first jaw 78 can have zero, one, or more (e.g., two) tissue guides 962. The second jaw 80 can have zero, one, or more (e.g., two) tissue guides 962. For example, FIGS. 1-7E illustrate that the jaws 801 can have tissue guides 962 (e.g., labeled in FIGS. 1 and 2A), for example, two tissue guides 962 as shown in FIGS. 1-7E. FIGS. 1-7E illustrate, for example, that one of the two tissue guides 962 can be on a first lateral side of the jaws 801 and that one of the two tissue guides 962 can be on a second lateral side of the jaws 801.

The tissue guides 962 can be tapered surfaces that can guide tissue between the jaws 801, for example, as the jaws 801 are advanced by the user against tissue that the user would like to pass suture through. The tissue guides 962 can be a tapered distal tips of the one or both of the jaws 801. For example, the tissue guides 962 can be a tissue guide angle of about 15 degrees to about 75 degrees, or more narrowly, from about 30 degrees to about 60 degrees, including every 1 degree increment within these ranges (e.g., 15 degrees, 30 degrees, 45 degrees, 60 degrees, 75 degrees). The tissue guide angle can be measured, for example, relative to the device longitudinal axis 956 (e.g., see FIG. 14A). The tissue guides 962 can be ramps for tissue that tissue can slide against. The tissue guides 962 can have the same or different angle as each other. For example, FIGS. 1-7E illustrate that the two tissue guides 962 that extend from the second jaw 80 can each have the same tissue guide angle.

The tissue guides 962 can be attached to or integrated with the first jaw 78 and/or with the second jaw 80. For example, FIGS. 1-7E illustrate that the tissue guides 962 can be integrated with the second jaw 80. The tissue guides 962 can extend from the first jaw 78 and/or from the second jaw 80. For example, FIGS. 1-7E illustrate that the tissue guides 962 can extend from the second jaw 80.

FIG. 1 illustrates that the two tissue guides 962 can form a split ramp with a space 964 between the tissue guides 962. The split ramp can guide tissue into the space between the jaws 801, which can be helpful for a tight cuff (e.g., tight rotator cuff), for example, when the rotator cuff is tight over the humerus which can make it more difficult to place the rotator cuff between the jaws 801 without the tissue guides 962 or without the split ramp. The split ramp can provide space for the suture 70 to move into and out of. For example, the suture 70 can be movable into and out of the space 964 as the shuttle is pushed into and out of the second jaw 80, respectively. The tissue guide 962 can be offset from the device longitudinal axis 956 such that the space 964 can be between the tissue guides 962. For example, FIG. 1 illustrates that a first lateral side of the second jaw 80 can have one of the tissue guides 962 and that a second lateral side of the second jaw 80 can have one of the tissue guides 962.

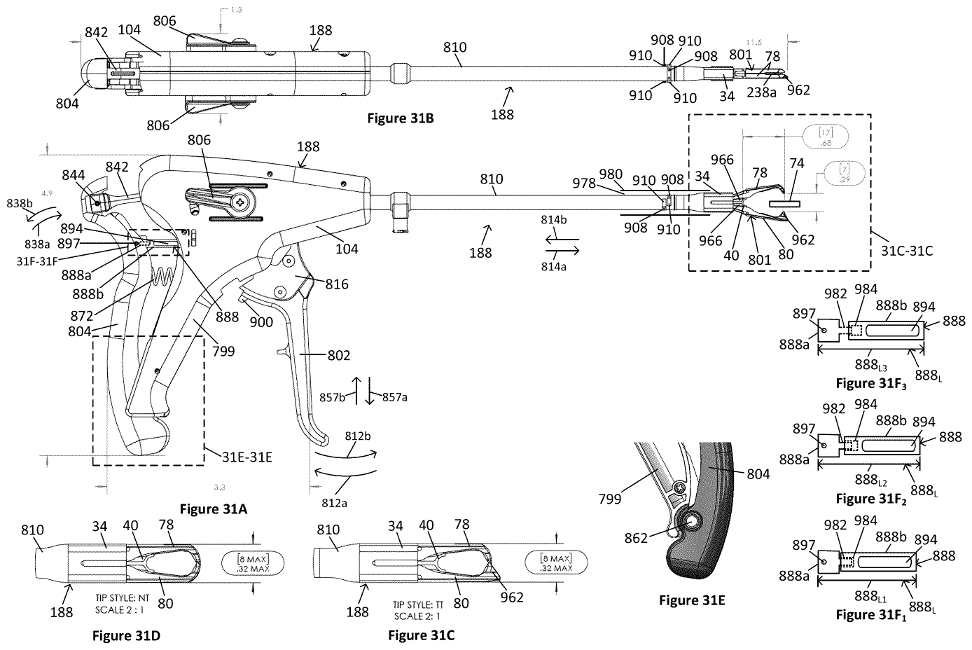

As another example, the jaws 801 can have either one of the tissue guides 962 shown in FIGS. 1-7E (e.g., see FIGS. 27A, 27B, and 31A-31C). For example, the jaws 801 can have only the tissue guide 962 on a first lateral side of the jaws 801 or can have only the tissue guide 962 on a second lateral side of the jaws 801. The tissue guides 962 can help the jaws 801 that a tissue guide 962 (e.g., the second jaw 80 in FIGS. 1-7E and the second jaw 80 in FIGS. 27A and 27B) to slide under the tissue. As another example, the first jaw 78 and the second jaw 80 can each have two tissue guides 962. In such variations, either the first jaw 78 or the second jaw 80 can be used to slide tissue into the space between the jaws 801, and in such variations, either the first jaw 78 or the second jaw 80 can be used as a bottom jaw and either the first jaw 78 or the second jaw 80 can be used as a top jaw.

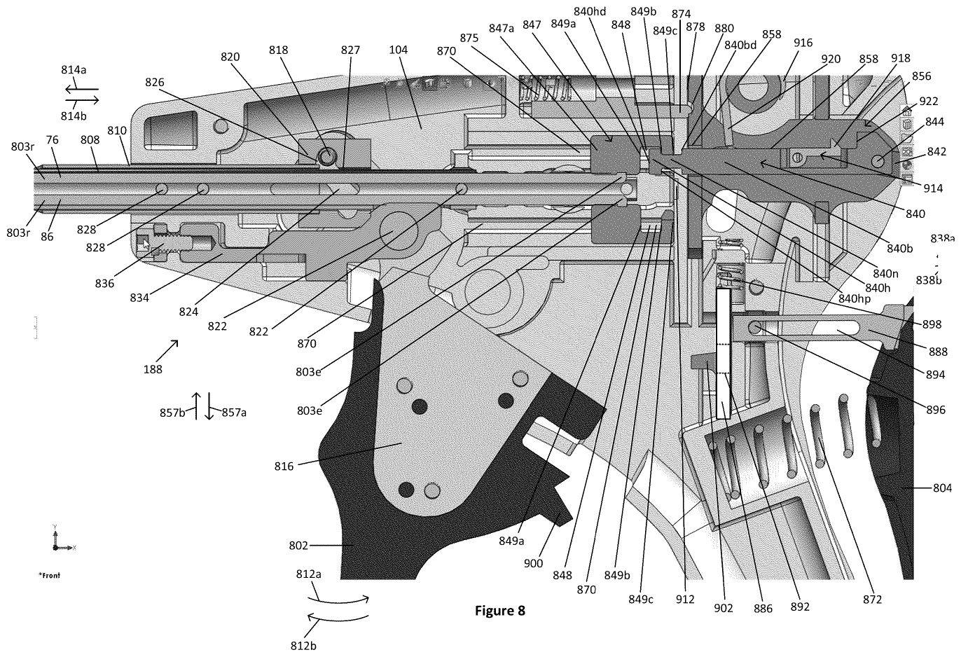

FIG. 8 illustrates mover 840 can have, for example, a mover head 840h, a mover neck 840n, and a mover body 840b. The mover head 840h can be a rim. The mover head 840h can be a collar. The mover head 840h can be a flange. The mover head 840h can be movable into and out of the groove 848 of the first engager 847a. The mover head 840h can be movable into and out of the groove 848 of the second engager 847b. The mover neck 840n can be narrower than the mover head 840h. The mover neck 840n can be narrower than the mover body 840b. The mover neck 840n can be a groove (e.g., a circumferential groove) around the mover 840. The mover neck 840n can be, for example, a groove (e.g., a circumferential groove) around the perimeter of a distal end of the mover body 840b. The mover neck 840n can be between the mover head 840h and the mover body 840b so that mover 840 can fit over the proximal end of the engagers 847 when the mover 840 is being moved into the grooves 848. The mover neck 840n can allow the mover 840 to fit over the proximal end of the engagers 847 so that the mover head 840h can be moved into the grooves 848. The mover neck 840n can allow the mover head 840h to push and pull the engagers 847.

FIG. 8 illustrates that the engagers 847 can have a first contact point 849a and a second contact point 849b. The first contact point 849a can be opposite the second contact point 849b. The first contact point 849a can be distal the second contact point 849b. The first contact point 849a can be a first contact surface of the engagers 847. The second contact point 849b can be a second contact surface. The first contact point 849a can be parallel to the second contact point 849b of the engagers 847. The first contact point 849a and the second contact point 849b can define the groove 848 of the engagers 847. For example, the first contact point 849a can define a distal end of the groove 848 and the second contact point 849b can define a proximal end of the groove 848. The mover 840 can contact and push against the first contact point 849a when the mover 840 is moved (e.g., advanced) in direction 814a. The mover 840 can contact and push against the second contact point 849b when the mover 840 is moved (e.g., retracted) in direction 814b.

FIG. 8 illustrates that when the mover 840 (e.g., the mover head 840h) is in the groove 848 of the first engager 847a and the mover 840 is moved in direction 814a, the mover 840 can contact and push against the first contact point 849a of the first engager 847a to advance the first pusher 76. For example, a distal end 840hd of the mover head 840h can contact and push against the first contact point 849a of the first engager 847a. As another example, when the mover 840 (e.g., the mover head 840h) is in the groove 848 of the first engager 847a and the mover 840 is moved in direction 814a, the distal end 840hd of the mover head 840h can contact and push against the first contact point 849a of the first engager 847a, a distal end 840bd of the mover body 840b can contact and push against a third contact point 849c of the first engager 847a, or both. The distal end 840bd of the mover body 840b can be, for example, a distal terminal end of the mover 840 proximal the mover neck 840n. The third contact point 849c of the first engager 847a can be proximal the second contact point 849b. The third contact point 849c of the first engager 847a can be a surface of the first engager 847a. FIG. 8 illustrates that when the mover 840 (e.g., the mover head 840h) is in the groove 848 of the first engager 847a and the mover 840 is moved in direction 814b, the mover 840 can contact and push against the second contact point 849b of the first engager 847a to retract the first pusher 76. For example, a proximal end 840hp of the mover head 840h can contact and pull against the second contact point 849b of the first engager 847a.

FIG. 8 illustrates that when the mover 840 (e.g., the mover head 840h) is in the groove 848 of the second engager 847b and the mover 840 is moved in direction 814a, the mover 840 can contact and push against the first contact point 849a of the second engager 847b to advance the second pusher 86. For example, the distal end 840hd of the mover head 840h can contact and push against the first contact point 849a of the second engager 847b. As another example, when the mover 840 (e.g., the mover head 840h) is in the groove 848 of the second engager 847b and the mover 840 is moved in direction 814a, the distal end 840hd of the mover head 840h can contact and push against the first contact point 849a of the second engager 847b, the distal end 840bd of the mover body 840b can contact and push against a third contact point 849c of the second engager 847b, or both. The third contact point 849c of the second engager 847b can be proximal the second contact point 849b. The third contact point 849c of the second engager 847b can be a surface of the second engager 847b. FIG. 8 illustrates that when the mover 840 (e.g., the mover head 840h) is in the groove 848 of the second engager 847b and the mover 840 is moved in direction 814b, the mover 840 can contact and push against the second contact point 849b of the second engager 847b to retract the second pusher 86. For example, the proximal end 840hp of the mover head 840h can contact and pull against the second contact point 849b of the second engager 847b.

FIG. 8 illustrates that an extension 912 can be between the first engager 847a and the second engager 847b. The extension 912 can be, for example, a tab. The extension 912 can be attached to or integrated with the housing. The mover neck 840n can be movable in directions 857a and 857b over the extension 912. The mover body 840b may not be movable in directions 857a and 857b over the extension 912. Such an arrangement can inhibit or prevent the mover 840 from becoming disengaged with the engagers 847 when the mover 840 is in an advanced position, for example, when the second trigger 804 is in an actuated position (e.g., when the second trigger 804 is squeezed). Such an arrangement can inhibit or prevent the mover 840 from becoming disengaged with the engagers 847 when the mover 840 is in an advanced position, for example, when the mover body 840b is distal the extension 912 (e.g., for reference, FIG. 8 shows the mover body 840b proximal the extension 912). Such an arrangement can releasably lock the selector 806 in the selector first position or in the selector second position when the second trigger 804 is in an actuated position (e.g., when the second trigger 804 is squeezed) and the selector 806 is in the selector first position or in the selector second position, respectively.

When the mover 840 is in a retracted position (e.g., when the second trigger 804 is in the second trigger first position), the mover 840 can be movable in directions 857a and 857b past the extension 912. When the mover 840 is in a retracted position and the selector 806 is in the selector first position, the mover 840 can be moved in direction 857a from a first side of the extension 912 to a second side of the extension 912. When the mover 840 is in a retracted position and the selector 806 is in the selector second position, the mover 840 can be moved in direction 857b from the second side of the extension 912 to the first side of the extension 912. FIG. 8 illustrates that when the device 188 is upright, the first side of the extension 912 can be the space superior to the extension 912 and that the second side of the extension 912 can be the space inferior to the extension 912. FIG. 8 illustrates that when the mover neck 840n is aligned with (e.g., in the same plane as) the extension 912, the selector 806 can be moved in directions 857a and 857b to select which of the pushers 803 to engage with. When the mover body 840b is aligned with (e.g., in the same plane as) the extension 912, the selector 806 can be prevented from being moved in directions 857a and 857b.

When the mover 840 is in an advanced position (e.g., when the second trigger 804 is in the second trigger second position), the extension 912 can block the mover 840 from being moved in directions 857a and 857b. For example, FIG. 8 illustrates that when the mover neck 840n is distal the extension 912 such that the mover body 840b is aligned with (e.g., in the same plane as) the extension 912 and the mover 840 is engaged with the first engager 847a, the extension 912 can prevent the mover 840 from being moved in direction 857a by blocking the mover body 840b when the mover 840 is in an advanced position (e.g., when the second trigger 804 is in the second trigger second position). As another example, FIG. 8 illustrates that when the mover neck 840n is distal the extension 912 such that the mover body 840b is aligned with (e.g., in the same plane as) the extension 912 and the mover 840 is engaged with the second engager 847b, the extension 912 can prevent the mover body mover 840 from being moved in direction 857b by blocking the mover body 840b when the mover 840 is in an advanced position (e.g., when the second trigger 804 is in the second trigger second position).

The extension 912 can help ensure that the mover 840 remains engaged with the same pusher during an advancement and retraction cycle of one of the pushers 803. The extension 912 can help ensure that both the pushers 803 are fully retracted before the first pusher 76 or the second pusher 86 can be advanced. Both lateral sides of the handle 104 (of which only one lateral side is shown in FIG. 8) can have an extension 912 such that both extensions 912 can form a channel that the mover neck 840n can fit through but through which the mover body 840b may not be able to fit through. An extension 912 can extend from one or both lateral sides of the handle 104.

FIG. 8 illustrates that an indicator 914 can provide auditory and/or tactile feedback to the user when the pushers 803 reach a fully advanced position by the second trigger 804 (e.g., when the second trigger 804 reaches the second trigger second position). The indictor 914 can have, for example, a spring 916 and a spur 918. The spring 916 can be, for example, a torsion spring. The spring 916 can be attached to the mover 856. The spur 918 can be attached to or integrated with the mover 840. The spur 918 can be an extension that extends from the mover 840. The spur 918 can be, for example, metal and the mover 840 can be, for example, plastic. The spur 918 can be moved by moving the mover 840. The spur 918 can move in unison with the mover 840. For example, the spur 918 can be moved in direction 814a when the second trigger 804 moves in direction 838a and can be moved in direction 814 when the second trigger 804 moves in direction 838b. The spur 918 can be movable (e.g., longitudinally movable) relative to the spring 916. The spur 918 can be slidable past (e.g., under) the spring 916. For example, the spur 918 can be slidable past (e.g., under) a spring arm 920 of the spring 916 in directions 814a and 814b. The spur 918 can be moved in direction 814a to push the spring arm 920 in direction 814a. As the first pusher 76 or the second pusher 86 nears a fully advanced position, the spur 918 can push the spring arm 920 in direction 814a. This can load the spring arm 920 in direction 814a. When the first pusher 76 or the second pusher 86 reaches a fully advanced position, the spur 918 can be pushed past the spring arm 920 at which point the spring arm 920 can unload (e.g., flip off) the spur 918 in direction 814b and hit the mover 840 (e.g., hit a surface 922 of the mover 840) or hit the mover 856. The contact of the spring arm 920 with the mover 840 or with the mover 856 can create audible and/or tactile feedback that can let the user know the second trigger 804 has been fully actuated (e.g., fully squeezed). For example, as the spur 918 is moved (e.g., slid) forward in direction 814a, the spur 918 can hit the spring arm 920 and load it up in direction 814a so that as the user continues to squeeze the second trigger 804, the spring 916 can load up until the spring arm 920 flips off the spur 918 and contacts the mover 840 (e.g., the surface 922 of the mover 840). This can provide an audible click and/or a tactile click letting the user know that they have pushed the pusher (e.g., first pusher 76 or second pusher 86) all the way forward.

FIG. 8 illustrates that the pins 828 can separate the pushers 803 from each other in the inner tube 808. The pins 828 can, for example, inhibit or prevent the pushers 803 from contacting each other as one of the pushers 803 is advanced and retracted. This can help ensure that while one of the pushers 803 (e.g., the first pusher 76) is advanced and retracted the other of the pushers 803 (e.g., the second pusher 86) can remain retracted.

FIG. 8 illustrates that a proximal end of the pushers 803 can have a rod 803r. The rod 803r can be, for example, a ram rod. FIG. 8 illustrates that the proximal end of the pushers can have an elbow 803e. FIG. 8 illustrates that the proximal terminal end of the pushers 803 can be attached to the engagers 847.

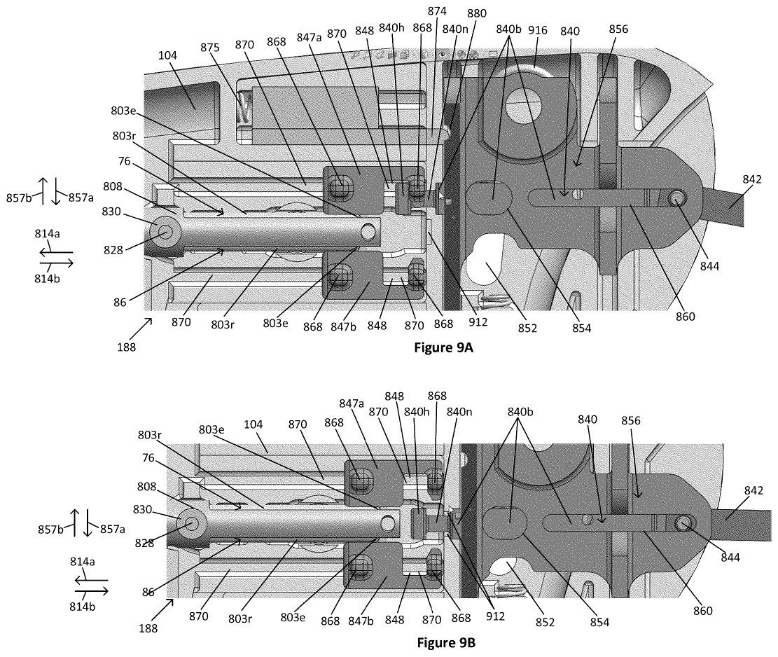

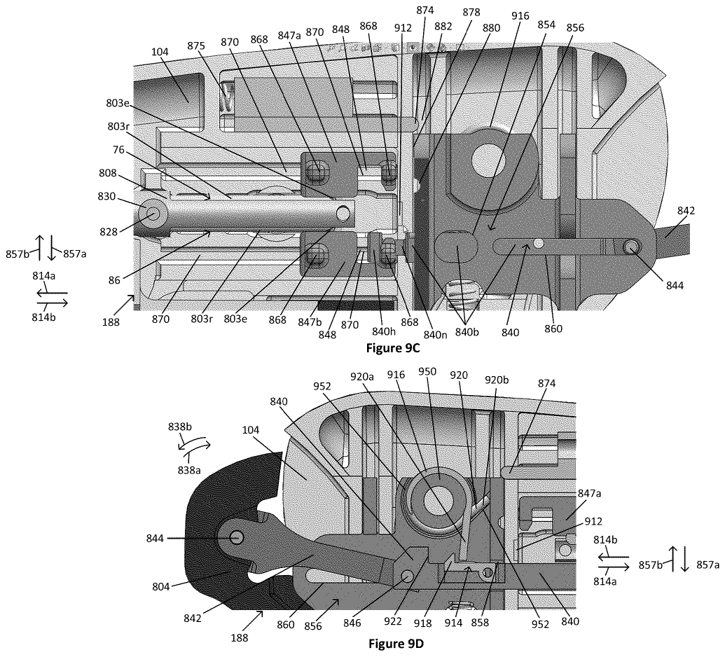

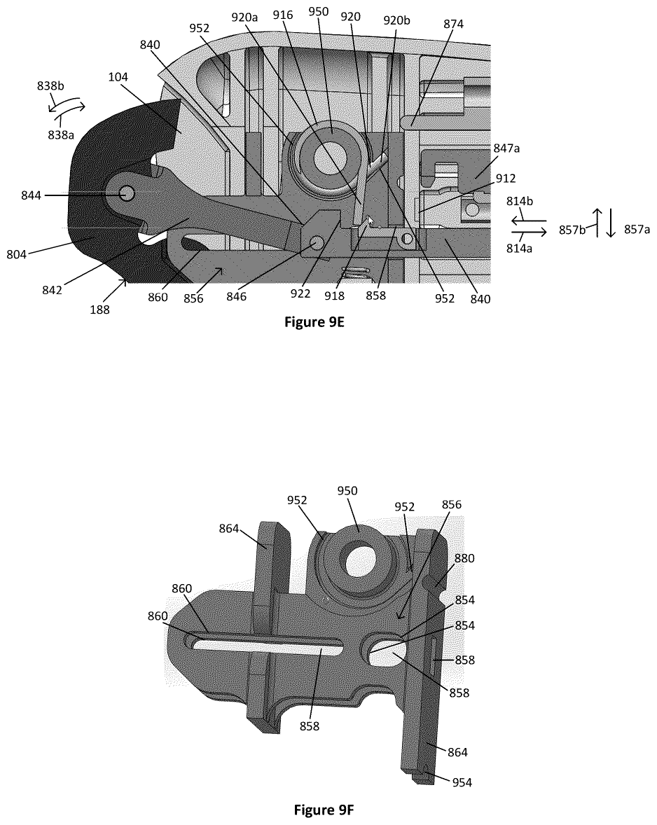

FIGS. 9A-9C illustrate that the mover 840 can be moved in directions 857a and 857b over the extension 912 when the mover 840 is in a fully retracted position. For example, FIGS. 9A-9C illustrate that the mover neck 840n can be moved in directions 857a and 857b over the extension 912 when the mover 840 is in a fully retracted position. The mover 840 can be in the fully retracted position when the second trigger 804 is in the second trigger first position. For example, from FIG. 9A to FIG. 9C, FIGS. 9A-9C illustrate that the mover neck 840n can be moved over the extension 912 in direction 857a, with FIG. 9B illustrating that the mover neck 840 can pass over the extension 912. As another example, from FIG. 9C to FIG. 9A, FIGS. 9C-9A illustrate that the mover neck 840n can be moved over the extension 912 in direction 857b, with FIG. 9B illustrating that the mover neck 840 can pass over the extension 912.

FIGS. 9D and 9E illustrate that when the mover 840 is in an advanced position, the extension 912 can block the mover 840 from being moved in directions 857a and 857b. FIG. 9D illustrates the mover 840 in a partially advanced position. FIG. 9E illustrates the mover 840 in a fully advanced position. FIGS. 9D and 9E illustrate that when the mover 840 is in a partially or fully advanced position, the mover neck 840n can be distal the extension 912 and the mover body 840b can be aligned with (e.g., in the same plane as) the extension 912 such that the extension 912 can inhibit the mover 840 from being moved in directions 857a and 857b by blocking the mover body 840b. For example, FIGS. 9D and 9E illustrate that when the mover 840 is in the groove 848 of the second engager 847b and the mover 840 is in an advanced position, the extension 912 can inhibit the mover 840 from being moved in direction 857b.

FIGS. 9D and 9E illustrate that the spring 916 can have two spring arms 920, for example, a spring first arm 920a and a spring second arm 920b. The spring first arm 920a can be the spring arm 920 referred to above that the spur 918 is engageable with. The spring first arm 920a can be movable relative to the spring second arm 920b. For example, when the spur 918 loads the spring 916 by pushing through the spring first arm 920a in direction 814a, the spring first arm 920a can be moved in direction 814a relative to the spring second arm 920b. The spur 918 can be moved in directions 814a and 814b by (e.g., under) the spring arm 920. For example, FIGS. 9D and 9E illustrate that the spur 918 can be moved in direction 814a through (e.g., under) the spring first arm 920a. As the spur 918 moves through (e.g., under) the spring first arm 920a in direction 814a, the spur 918 can push the spring first arm 920a in direction 814a. FIG. 9D illustrates the spring first arm 920a in a pre-loaded configuration. FIG. 9E illustrates the spring second arm 920b in a post-loaded configuration, for example, after the spring 920 has flipped off the spur 918 and contacted the mover 840 (e.g., hit the surface 922 of the mover 840). As another example, from FIG. 9E to FIG. 9D, FIGS. 9E and 9D illustrate that the spur 918 can be moved in direction 814b through (e.g., under) the spring first arm 920a. As the spur 918 moves through (e.g., under) the spring first arm 920a in direction 814b, the spring first arm 920a can slide over the spur 918. FIG. 9D illustrates the spring first arm 920a in a non-loaded configuration after the mover 840 has been retracted past the spring first arm 920a. FIGS. 9A-9E illustrate that the spur 918 can have the shape shown such that the spring first arm 920a can slide over the spur easier when the mover 840 is moved in direction 814b than when the mover 840 is moved in direction 814a.

FIGS. 9D and 9E illustrate that the spring 916 can sit on or over a boss 950, in a recess 952, or both over the boss 950 and in the recess 952. FIGS. 9D and 9E illustrate that the coil of the spring 916 can be in a first portion of the groove 952 and that spring second arm 920b can be a second portion of the groove 952.

FIG. 9F illustrates that the mover 856 can have any arrangement of the features shown. For example, FIG. 9F illustrates that the channel 858 can extend between the two channels 860 and can extend between the two openings 854. FIG. 9F illustrates that the mover can have a recess 954. The recess 954 can be, for example, a groove. The lockout bar 888 can fit in the recess 954. For example, FIGS. 2C and 3C illustrate that lockout bar 888 engaged with the recess 954. The recess 954 can, for example, increase the range of the mover 856 in direction 857a. The recess 954 can, for example, guide the lockout bar 888 into the recess 904. The recess 954 can, for example, inhibit or prevent the end of the protrusion 864 from blocking movement of the lockout bar 888 in direction 814a. The recess 954 can, for example, inhibit or prevent the distal end of the lockout bar 888 (e.g., the end that is closer to the jaws 801) from getting hung up on the protrusion 864 when the second trigger 804 is squeezed. As another example, when the lockout bar 888 is engaged with the recess 954 (e.g., as shown in FIGS. 2C and 3C), the recess 954 can inhibit or prevent lateral movement (e.g., movement into and out of the page as shown in FIGS. 2C and 3C) of the distal end of the lockout bar 888, which can give the device a more stable feel.

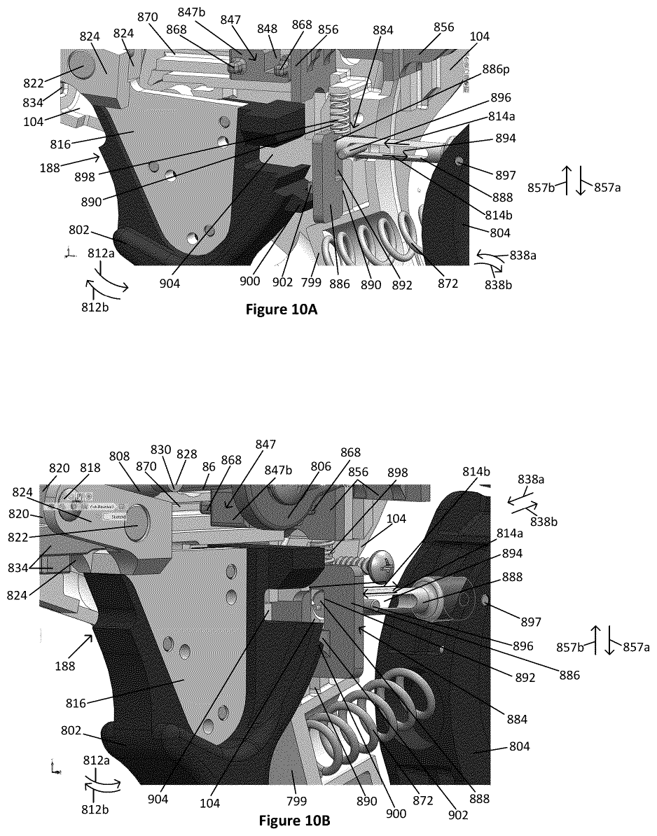

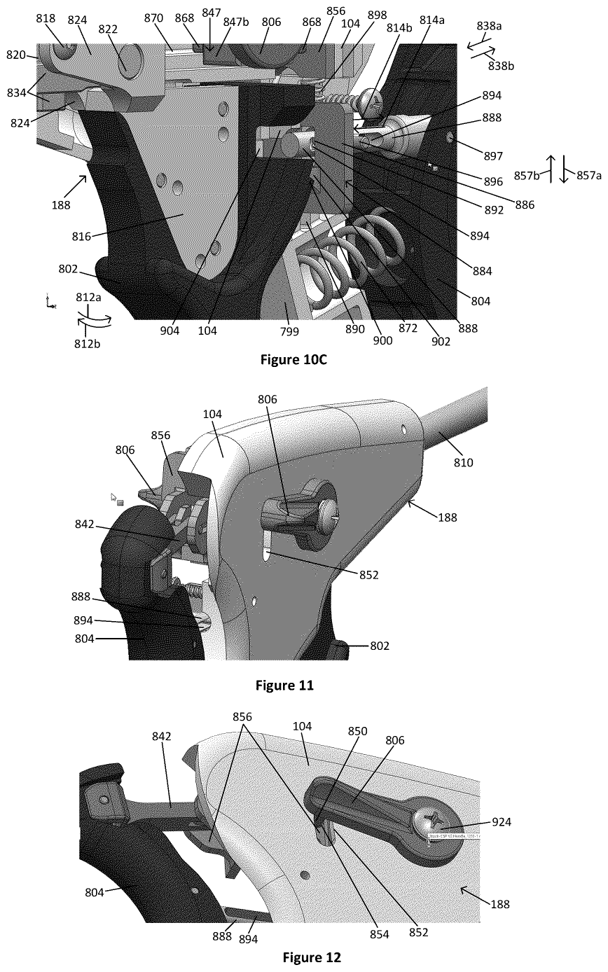

FIG. 10A illustrates that when the lockout gate 886 is in a lockout position, the lockout bar 888 may not be movable in direction 814a. For example, FIG. 10A illustrates that when the lockout gate 886 is in a lockout position, the lockout bar 888 can be aligned with a proximal surface 886p of the lockout gate 886 such that the proximal surface 886p can block the lockout bar 888 from moving in direction 814a. This can lock the second trigger 804 in the second trigger first position. The lockout gate 886 can be biased by the lockout spring 898 to be in the lockout position (e.g., the position shown in FIG. 10A). The lockout position of the lockout gate 886 can be a lowered position of the lockout gate 886. The lockout position of the lockout gate 886 can be a closed position of the lockout gate 886. FIG. 10A illustrates the first trigger 802 in an advanced position (e.g., having been advanced in direction 812a) just before the extension 900 contacts the extension 902. FIG. 10A illustrates an exemplary lockout configuration of the lockout 884.

FIG. 10B illustrates that when the lockout gate 886 is in a non-lockout position, the lockout bar 888 can be movable in direction 814a. For example, FIG. 10B illustrates that when the lockout gate 886 is in a non-lockout position, the lockout bar 888 can be aligned with the opening 892. FIG. 10B illustrates that the first trigger 802 (e.g., by pushingthe extension 902) can move the lockout gate 886 to the non-lockout position, for example, by moving the lockout gate 886 in direction 857b, for example, by pushing on the extension 902 in direction 857b.

FIG. 10C illustrates that when the lockout gate 886 is in a non-lockout position, the lockout bar 888 can be movable in directions 814a and 814b in the opening 892 as the second trigger moves in directions 838a and 838b, respectively. FIG. 10C illustrates, for example, the lockout bar 888 in the opening 892 and in the recess 904.

From FIG. 10A to FIG. 10C, FIGS. 10A-10C illustrate that the lockout gate 886 can be moved in direction 857b (e.g., can be raised by the first trigger 802) to align the opening 892 with the lockout bar 888 and that the lockout bar 888 can move through the opening 892 into the recess 904 in direction 814a when the second trigger 804 is actuated (e.g., when the second trigger 804 is pushed in direction 838a). FIGS. 10A-10C illustrate an exemplary process of unlocking the lockout 884.

From FIG. 10C to FIG. 10A, FIGS. 10C-10A illustrate that the lockout bar 888 can move out of the recess 904 and through the opening 892 in direction 814b when the second trigger 804 moves or is moved in direction 838b and that the lockout gate 886 can be moved in direction 857a (e.g., can be lowered by the lockout spring 898) to misalign the opening 892 and the lockout bar 888. FIGS. 10C-10A illustrate an exemplary process of locking the lockout 884.

FIGS. 10A-10C illustrate that the connector 816 can have two arms 824, with one on each side of the mover 820 such that one of the two connectors 818 can extend into one of the two channels 826 on a first side of the mover 820 and such that the other of the two connectors 818 can extend into the other of the two channels 826 on a second side of the mover 820.

FIG. 11 illustrates that the device can have two selectors 806. The two selectors 806 can be independent of each other. As another example, FIG. 11 illustrates that moving one of the selectors 806 can move the other selector 806 as well (e.g., via the mover 856). The left lateral half of the handle 104 is shown transparent in FIG. 11 for illustrate purposes so that both selectors 806 can be seen.

FIG. 12 illustrates the extension 850 can extend from the selectors 806, through the opening 852 in a side of the handle 104, and into the opening 854 in the mover 856. The extension 850 can be attached to or integrated with the selectors 806. FIG. 12 illustrates that the selector 806 can be attached to the handle 104 with a connector 924. The connector 924 can be, for example, a screw.

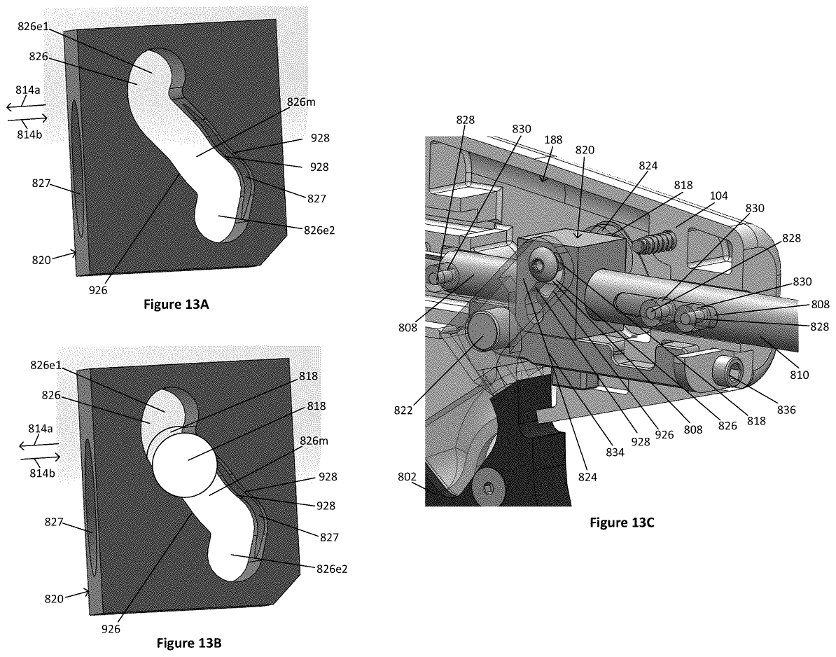

FIG. 13A illustrates that the channel 827 can extend through the mover 820. For example, FIG. 13A illustrates that the channel 827 can extend longitudinally through the mover 820.

FIG. 13A illustrates that the channel 826 can extend through the mover 820. For example, FIG. 13A illustrates that the channel 826 can extend transversely through the mover 820.

FIG. 13A illustrates that the channel 826 can intersect with the channel 827.

FIG. 13A illustrates that the channel 826 can be a single channel that can extend through the mover 820 from a first side (e.g., a first lateral side) to a second side (e.g., a second lateral side). As another example, FIG. 13A illustrates that the mover 820 can have two channels 826, for example, a first channel 826 on a first side (e.g., a first lateral side) of the mover 820 and a second channel 826 on a second side (e.g., a second lateral side) of the mover.

FIG. 13A illustrates that the channel 826 can have the shape shown. FIG. 13A illustrates that the channel 826 can be asymmetrical. For example, FIG. 13A illustrates that the channel 826 can be an S-shaped slot or a backwards S-shaped slot. The mover 820 can provide motion and can provide dwell. For example, the ends of the channel 826 (e.g., the ends of the S-shape) can provide dwell and the middle of the channel 826 (e.g., the middle of the S-shape) can provide motion. For example, the mover 820 and the connectors 818 can be a dwell mechanism such that the first end 82e1 and the second end 82e2 of the channel 826 can be holding or dwell segments of the channel 826 and such that middle 826m of the channel 826 can be the motion segment of the channel 826. The first end 82e1 of the channel 826 can be, for example, a first end of the S-shape slot. The second end 82e2 of the channel 826 can be, for example, a second end of the S-shaped slot. The middle 826m of the channel 826 can be between the first and second ends 826e1, 826e2.

When the connector 818 is in the first end 82e1 of the channel 826, further movement of the first trigger 802 in direction 812b may not move the mover 820 in direction 814a or 814b such that the mover 820 can remain stationary as the first trigger 802 is moved further in direction 812b. This can prevent the first trigger 802 from exerting too much force on the mover 820 in direction 814b. Thus, when the connector 818 is in the first end 82e1 of the channel 826, the user can continue to move the first trigger 802 in direction 812b without making the jaws 801 open any further. As another example, when the connector 818 is in the first end 82e1 of the channel 826, further movement of the first trigger 802 in direction 812b can move the mover 820 in direction 814a as the first trigger 802 is moved further in direction 812b. This can prevent the first trigger 802 from exerting too much force on the mover 820 in direction 814b.

When the connector 818 is in the second end 82e2 of the channel 826, further movement of the first trigger 802 in direction 812a may not move the mover 820 in direction 814a or 814b such that the mover 820 can remain stationary as the first trigger 802 is moved further in direction 812a. This can prevent the first trigger 802 from exerting too much force on the mover 820 in direction 814a and/or can allow the maximum clamp force exerted by the jaws 801 to be controlled, for example, by preventing the jaws 801 from getting closer to each other than the gap 906 when the jaws 801 are in a fully closed configuration. Thus, when the connector 818 is in the second end 82e2 of the channel 826, the user can continue to move the first trigger 802 in direction 812a without making the jaws 801 close any further. The channel 826 can thereby provide a little bit of dwell in the last bit of trigger stroke in direction 812a (e.g., last 10 degrees or less, last 15 degrees or less) which can let the user really squeeze the first trigger 802 without making the mover 820 travel further forward to further close the jaws 801. As another example, when the connector 818 is in the second end 82e2 of the channel 826, further movement of the first trigger 802 in direction 812a can move the mover 820 in direction 814b as the first trigger 802 is moved further in direction 812a. This can prevent the first trigger 802 from exerting too much force on the mover 820 in direction 814a.

When the connector 818 is in the middle 826m of the channel 826 and the first trigger 802 is moved in direction 812a, the connector 818 can slide against a guide 926 which can cause the mover 820 to move in direction 814a. The guide 926 can be an edge and/or surface of the mover 820. For example, FIG. 13A illustrates that the guide 926 can be an inner distal surface of the mover 820 that defines the channel 826.

When the connector 818 is in the middle 826m of the channel 826 and the first trigger 802 is moved in direction 812b, the connector 818 can slide against a guide 928 which can cause the mover 820 to move in direction 814b. The guide 928 can be an edge and/or surface of the mover 820. For example, FIG. 13A illustrates that the guide 928 can be an inner proximal surface of the mover 820 that defines the channel 826.

FIG. 13A illustrates that the guide 926 can be a first guide and that the guide 928 can be a second guide. FIG. 13A illustrates that the guide 926 can be a front face of the channel 826 and that the guide 928 can be a rear face of the channel 826. FIG. 13A illustrates that the guide 926 can face the guide 928. FIG. 13A illustrates that the guide 928 can face the guide 926. FIG. 13A illustrates that the guide 926 can be opposite the guide 928.

FIG. 13A illustrates that the guides 926 and 928 can be curved and/or can have undulations. The guides 926 and 928 can be curved and/or can have undulations, for example, to help smooth out the travel of the connector 818 in the channel 826. As another example, the guides 926 and 928 can be straight, non-curved, flat, or any combination thereof.

FIG. 13B illustrates that two connectors 818, for example, a first connector 818 and a second connector 818 can link the first trigger 802 to the mover 820. The connectors 818 can sit in the channel 826 or in two separate channels 826. The connectors 818 can translate in the channel 826 or in two separate channels as the first trigger 802 is moved in directions 812a and 812b.

FIG. 13C illustrates that there can be two connectors 818 that can extend into the channel 826. FIG. 13C illustrates, for example, that the connectors 818 can extend into the channel 826 and ride against the guides 926 and 928. The connector 816 is shown transparent for illustrative purposes so that the connector 818 on each side of the mover 820 can be seen. FIG. 13C illustrates that the mover 820 can move in directions 814a and 814b over the trigger adjust yoke 834. The connectors 830 in FIG. 13C are shown transparent so that the pins 828 can be more easily seen.

FIG. 14A illustrates the first jaw 78 can have a track 64 and that the second jaw 80 can have a track 66. The tracks 64 and 66 can be tracks for the shuttle 14, can be tracks for the pushers 803, or both. The shuttle 14 and the first pusher 76 can be, for example, translatable (e.g., slidable) in the track 64 so that the first pusher 76 can push the shuttle 14 from the first jaw 78 to the second jaw 80. The shuttle 14 and the second pusher 86 can be, for example, translatable (e.g., slidable) in the track 66 so that the second pusher 86 can push the shuttle 14 from the second jaw 80 to the first jaw 78. The track 64 can also be referred to, for example, as the first track 64, the first jaw track 64, the shuttle first track 64, the first shuttle track 64, the pusher first track 64, the first pusher track 64, the jaw track 64, the first jaw track 64, or variations thereof. The track 66 can also be referred to, for example, as the second track 66, the second jaw track 66, the shuttle second track 66, the second shuttle track 66, the pusher second track 66, the second pusher track 66, the jaw track 66, the second jaw track 66, or variations thereof.

FIG. 14A illustrates that the first and second jaws 78, 80 can have slots 238. For example, FIG. 14A illustrates that the first jaw can have a first slot 238a and that the second jaw 80 can have a second slot 238b. The suture 70 can be movable in the slots 238. The shuttle 14 (e.g., the suture holder 18 and/or male stops) can be movable in the slots 238. The suture 70 and the shuttle 14 can be movable in the slots 238. The suture holder 18 may or may not extend into the slots 238. For example, FIG. 14A illustrates that the suture holder 18 may not extend into the slots 238. The slots 238 can extend along the outer surface of the first and second jaws 78, 80.

For example, the first slot 238a is labeled twice in FIG. 14A to illustrate that that the first slot 238a can extend from the tip of the first jaw 78 along a length of the first jaw 78. For example, the second slot 238b is labeled twice in FIG. 14A to illustrate that that the second slot 238b can extend from the tip of the second jaw 80 along a length of the second jaw 80. The slots 238 can extend from the outer surface of the first and second jaws 78, 80 to the track that is in the jaws 801. For example, the first slot 238a can extend from the outer surface of the first jaw 78 to the first track 64, and the second slot 238b can extend from the outer surface of the second jaw 80 to the second track 66. The slots 238 can extend medially along the outer surface of the first and second jaws 78, 80. The suture 70 can be accessible through or extend out of the slots 238.

FIG. 14A illustrates that the first and second pushers 76, 86 can have a shuttle seat 274. The shuttle seat 274 can be a distal terminal end of the first and second pushers 76, 86. The shuttle seat 274 can be an inverse shape to the shape of the shuttle tip 164. For example, if the shuttle tip 164 has an angled end, the shuttle seat 274 can have the opposite angle. If the shuttle tip 164 has a convex curved end, the shuttle seat 274 can have a concave curved end with the same radius of curvature as the shuttle tip 164.

FIG. 14A illustrates that the inner tube 808 and the jaws 801 can be attached to each other via a connector 930. The connector 930 can be, for example, a jaw mount neck. The jaws 801 can be attached to the connector 930, for example, via pins 932. As the jaws 801 are opened and closed, the jaws 801 can rotate about the pins 932.

FIG. 14A illustrates that the connector 930 can have a first track 934 for the first pusher 76 and a second track 936 for the second pusher 86. The first pusher 76 can be translatable (e.g., slidable) in the first track 934. The second pusher 86 can be translatable (e.g., slidable) in the second track 936. The first track 934 can guide the first pusher 76 in the connector 930. The second track 936 can guide the second pusher 86 in the connector 930.

FIG. 14A illustrates that a guide 938 can be attached to the connector 930. As another example, the guide 938 can be attached to the opener 40 and/or to the opener 34. The guide 938 can guide the first pusher 76 into the first shuttle track 64 and can guide the second pusher 86 into the second shuttle track 66. The guide 938 can inhibit or prevent the pushers 803 from prolapsing out into the space (e.g., visually shown as the space 960 marked by the dashed oval in FIG. 14A) between the jaws 801 instead of translating into the first and second shuttle tracks 64, 66 as the pushers 803 are advanced by the second trigger 804. The guide 938 can, for example, inhibit or prevent the pushers 803 from buckling between the connector 930 and the jaws 801.

The guide 938 can guide the first pusher 76 into the first shuttle track 64, for example, from a retracted position. When the first pusher 76 is in a retracted position (e.g., a fully retracted position), the first pusher 76 can be distal the distal terminal end of the guide 938 and/or can be distal the proximal terminal end of the first shuttle track 64 in the first jaw 78. The proximal terminal end of the first shuttle track 64 in the first jaw 78 can be, for example, the entrance of the first shuttle track 64 at the proximal end of the first jaw 78. For example, FIG. 14A illustrates that when the first pusher 76 is in a retracted position (e.g., a fully retracted position), the distal terminal end of the first pusher 76 (e.g., the shuttle seat 274 of the first pusher 76) can be distal the distal terminal end of the guide 938 and can be distal the proximal terminal end of the first shuttle track 64. As another example, when the first pusher 76 is in a retracted position (e.g., a fully retracted position), the first pusher 76 can be proximal the distal terminal end of the guide 938 and/or can be proximal the proximal terminal end of the first shuttle track 64 in the first jaw 78. For example, when the first pusher 76 is in a retracted position (e.g., a fully retracted position), the distal terminal end of the first pusher 76 (e.g., the shuttle seat 274 of the first pusher 76) can be proximal the distal terminal end of the guide 938 and can be proximal the proximal terminal end of the first shuttle track 64.

The guide 938 can guide the second pusher 86 into the second shuttle track 66, for example, from a retracted position. When the second pusher 86 is in a retracted position (e.g., a fully retracted position), the second pusher 86 can be distal the distal terminal end of the guide 938 and/or can be distal the proximal terminal end of the second shuttle track 66 in the second jaw 80. The proximal terminal end of the second shuttle track 66 in the second jaw 80 can be, for example, the entrance of the second shuttle track 66 at the proximal end of the second jaw 80. For example, FIG. 14A illustrates that when the second pusher 86 is in a retracted position (e.g., a fully retracted position), the distal terminal end of the second pusher 86 (e.g., the shuttle seat 274 of the second pusher 86) can be distal the distal terminal end of the guide 938 and can be distal the proximal terminal end of the second shuttle track 66. As another example, when the second pusher 86 is in a retracted position (e.g., a fully retracted position), the second pusher 86 can be proximal the distal terminal end of the guide 938 and/or can be proximal the proximal terminal end of the second shuttle track 66 in the second jaw 80. For example, when the second pusher 86 is in a retracted position (e.g., a fully retracted position), the distal terminal end of the second pusher 86 (e.g., the shuttle seat 274 of the second pusher 86) can be proximal the distal terminal end of the guide 938 and can be proximal the proximal terminal end of the second shuttle track 66.

FIG. 14A illustrates the pushers 803 in a fully retracted configuration.

FIG. 14A illustrates that the distal end of the guide 938 can be tapered such that surfaces 940 can guide the pushers 803 into the first and second shuttle tracks 64, 66. The surfaces 940 can bridge the gap between the first and second tracks 934, 936 in the connector 930 and the first and second shuttle tracks 64, 66 in the jaws 801. This can inhibit or prevent the pushers 803 from prolapsing out into the space (e.g., visually shown as the space 960 marked by the dashed oval in FIG. 14A) between the jaws 801 instead of translating into the first and second shuttle tracks 64, 66 as the pushers 803 are advanced by the second trigger 804. The guide 938 can, for example, inhibit or prevent the pushers 803 from buckling between the connector 930 and the jaws 801. The surfaces 940 can be angled to guide the pushers 803 into the first and second shuttle tracks 64, 66. The angle of the surfaces 940 can be, for example, less angled (e.g., by about 1 degree to about 10 degrees, including every 1 degree increment within this range) than the straight portion of the first and second shuttle tracks 64, 66 at a proximal end of each of the jaws 801 when the jaws 801 are in a closed configuration (e.g., the fully closed configuration shown in FIG. 14A), for example, as measured relative to the device longitudinal axis 956. The angle of the surfaces 940 can be, for example, less angled than the straight portion of the first and second shuttle tracks 64, 66 at a proximal end of each of the jaws 801 when the jaws 801 are in an open configuration (e.g., a fully open configuration), for example, as measured relative to the device longitudinal axis 956.

FIG. 14B illustrates that the first track 934 in the connector 930 can have a t-shape, and that the second track 934 in the connector 930 can have a t-shape.

FIG. 14B illustrates that the connector 930 can have a slot 942 for the opener 40. The opener 40 can be slidable in the slot 942, for example, as the opener 40 is moved in directions 814a and 814b by the first trigger 802.

FIG. 14C illustrates that the opener 40 can have extensions 944 that can be slidable in the slot 940 of the connector 930. The extensions 944 can be, for example, lateral extensions. FIG. 14C illustrates that the opener 40 can be movable in the slot 942 of the connector 930. The extensions 944 can be attached to (e.g., welded to) the outer tube 810. The extensions 944 can, for example, be attached to an inner surface of the outer tube 810. As another example, the extensions 944 can extend be extend through slots 958 in the outer tube 810 (e.g., as shown in FIG. 2A). The extensions 844 can be attached to the outer tube 810 in the slots 958.

The slots 958 in the outer tube 810 can streamline efficient assembly of the device 188. The opener 40 can be a flanged separator such that the extensions 944 can be flanges of the opener 40.

FIG. 14C illustrates that the each of the pushers 803 can have the rod 803r and a tape 803t. The rod 803r can be, for example, stainless steel, and the tape 803t can be, for example, Nitinol or cobalt chrome. The tape 803t can be a harder material than the shuttle 14. For example, the shuttle 14 can be Nitinol and the tape 803t can be cobalt chrome.

The rod 803r can be proximal end of the pushers 803 and the tape 803t can be distal end of the pushers 803. The shuttle seat 274 can be a distal terminal end of the tape 803t. FIG. 14C illustrates that the tape 803t can be attached to the rod 803r. FIG. 14C illustrates that a distal end of the rod 803r can have a slot that the tape 803t can be attached to or integrated with. The rod 803r can be integrated with the tape 803t. As another example, the rod 803r can be attached to (e.g., welded to) the tape 803t. FIG. 14C illustrates that the rod 803r and the tape 803t of the first pusher 76 can be slidable in the first track 934 of the connector 930. FIG. 14C illustrates that the rod 803r and the tape 803t of the second pusher 86 can be slidable in the second track 936 of the connector 930.

FIG. 14D illustrates that the opener 40 can have a slot 946. The guide 938 can be in the slot 946. The opener 40 can move relative to the guide 938. For example, as the opener 40 is moved in directions 814a and 814b by the first trigger 802, the opener 40 can move relative to the guide 938 such that the guide 938 can be movable in the slot 946. As another example, the guide 938 can be fixed to the opener 40 such that the opener and the guide 938 can be moved in unison when the opener 40 is moved in directions 814a and 814b by the first trigger 802.

FIG. 14D illustrates that the opener 40 can have a ridge 966 on first and second sides. The ridge 966 can be a rib. The opener 40 can be symmetrical about the longitudinal axis such that the ridge 966 on the second side is shown obstructed in FIG. 14D but can be a mirror image of the ridge 966 shown in FIG. 14D.

FIG. 14E illustrates that the guide 938 can be, for example, a pin. FIG. 14E illustrates that a proximal end 948 of the guide 938 can be attached to the connector 930, for example, can be inserted into a hole or channel in the connector 930.

FIG. 14F illustrates a variation of a fully closed configuration of the jaws 801 with the opener 40 shown transparent for illustrative purposes. FIG. 14F illustrates that one or both of the jaws 801 can have a groove 968. For example, FIG. 14F illustrates that both the first jaw 78 and the second jaw 80 can have a groove 968. FIG. 14F illustrates that the groove 968 in the first jaw 78 can be opposite the groove 968 in the second jaw 80.

The grooves 968 of the first and second jaws 78, 80 can be guides for the ridges 966 on the opener 40. The ridge 966 of the opener 40 can be translatable (e.g., slidable) in the groove 968. The ridges and grooves 966, 968 can keep the jaws 801 aligned with each other as the jaws 801 are opened and closed. The ridges and grooves 966, 968 can keep the jaws 801 aligned with the device longitudinal axis 956 as the jaws 801 are opened and closed. The jaws 801 (e.g., the tips of the jaws 801) can be centered with each other via the slidable arrangement between the ridges and grooves 966, 968. The jaws 801 (e.g., the tips of the jaws 801) can be centered with the device longitudinal axis 956 via the slidable arrangement between the ridges and grooves 966, 968. The ridges and groves 966, 968 can keep the jaws 801 aligned so that the first jaw track 64 is aligned with the second jaw track 66 when the jaws 801 are in the fully closed configuration. This can help prevent the shuttle 14 from missing the opposite jaw when the shuttle 14 is passed to the opposite jaw or from being partially blocked by the opposite jaw when the shuttle 14 is passed to the opposite jaw. The ridges and grooves 966, 968 can keep the jaws 801 aligned during opening of the jaws 801, during closing of the jaws 801, or during opening and closing of the jaws 801. The ridges and grooves 966, 968 can keep the jaws 801 aligned, for example, by inhibiting or preventing relative lateral movement of the first jaw 78 relative to the second jaw 80, or vice versa, as the jaws 801 or pushed and prodded against tissue. FIG. 14A illustrates the ridges and grooves 966, 968 engaged with each other.

FIG. 14F illustrates that the outer tube 810 can have two slots 958. FIG. 14F illustrates that the slots 958 can be lateral slots. FIG. 14F illustrates that the slots 958 can be opposite each other.

A portion of the distal end of the jaws 801 is shown transparent in FIG. 14F for illustrative purposes (e.g., the portion below dashed line 969).

FIG. 14G illustrates that when the jaws 801 are in the fully open configuration, a distal end of the ridges 966 can be engaged with a proximal end of the grooves 968.

FIG. 14G illustrates the portion of the distal end of the jaws 801 that is shown transparent in FIG. 14F for illustrative purposes (e.g., the portion below dashed line 969).

FIG. 14G illustrates two suture holders 908, each having two arms 910.

FIGS. 14F and 14G illustrate that a portion 423 of the shuttle tips can remain exposed outside of the jaws 801 when the shuttle 14 is fully translated into the jaws. The exposed portion 423 can have an exposed length, for example, from about 0.25 mm to about 5.00 mm or more, including every 0.25 mm increment within this range (e.g., 0.50 mm, 1.00 mm, 1.50 mm). The exposed portion 423 can align the first and second jaws 78, 80 when they close. The exposed portion 423 can pierce tissue when the first and second jaws 78, 80 are closed against each other and before the shuttle 14 is translated to the other jaw. This can advantageously leverage the clamping force of the jaws to cut tissue with the shuttle 14. A portion of the exposed portion 423 can pierce tissue when the shuttle 14 is translated from the lower jaw to the upper jaw, or vice versa.

FIGS. 14F and 14G illustrate that when the shuttle 14 is in the first jaw 78 or the second jaw 80, the distal tip of the shuttle 14 (e.g., the exposed portion 423) can protrude from the jaw that the shuttle 14 is in. This can prevent distal male stop 412 from being deflected by the jaw and creating a moment, if deflected, that can undesirably disengage the proximal male stop 412 from the female stop 416.

FIGS. 14F and 14G illustrate that the suture holder 18 may not extend into the slots 238 when the shuttle 14 is in the first and second jaws 78, 80.

FIG. 14H illustrates the two suture holders 908 shown in FIG. 14G. FIG. 14H illustrates, for example, that a center portion of the suture holders 908 can have a radius 970, and that the arms 910 can extend away from the central portion. The circle 972 in FIG. 14H is shown for illustrative purposes only to show how the arms 910 in FIG. 14G can extend away from the outer tube 810. FIG. 14I illustrates that the suture holder 18 can extend into the slots 238 when the shuttle 14 is in the first and second jaws 78, 80.

FIG. 14J illustrates that the first and second pushers 76, 80 can be advanced into the opposite jaw when pushing the shuttle 14. As another example, the first and second pushers 76, 86 may not be advanceable into the opposite jaw when pushing the shuttle 14. FIG. 14J illustrates that the shuttle seat 274 of the pushers 803 can have, for example, a V-shape (or A-shape) or a curved (e.g., U-shape).

FIG. 14K illustrates a variation of the device 188 in a fully open configuration with the suture 70 attached to the shuttle 14. FIG. 14K illustrates that the suture 70 can extend through the slots 238 when the shuttle 14 is in the jaws 801.

FIG. 15A illustrates that the shuttle 14 can have a shuttle body 160 and that the suture holder 18 can extend from the shuttle body 160. FIG. 15A illustrates that the suture holder 18 can extend longitudinally along the shuttle body 160, for example, along a length of the shuttle 14. As another example, the suture holder 18 can extend transversely across the shuttle body 160, for example, across a width of the shuttle 14 (e.g., perpendicularly across a width of the shuttle 14). FIG. 15A illustrates, for example, that the suture holder 18 can be an arc integral with the shuttle body 160. For example, the shuttle 14 can be made from a single panel of material (e.g., metal). The lateral sides of the suture holder 18 can be cut, and the longitudinal ends can remain integrated with the shuttle body 160. The suture holder 18 can then be bent or otherwise deformed away from the plane of the shuttle body 160, for example forming an arc away from the plane of the shuttle body 160. FIG. 15A illustrates that the shuttle 14 can have shuttle fingers 156 and shuttle slots 158 between the shuttle fingers 156. The shuttle fingers 156 can be flexible or rigid. The shuttle slots 158 can be, for example, lateral shuttle slots. The shuttle fingers 156 can extend from the shuttle body 160. As another example, the shuttle body 160 can include the shuttle fingers 156 such that the shuttle slots 158 can be, for example, slots in the shuttle body 160.

FIG. 15A illustrates that the suture 70 can be attached to the shuttle 14, for example, to the suture holder 18. The suture 70 can have a suture loop 162 at the terminal end of the suture 70. The suture loop 162 can extend around and completely or partially circumscribe the suture holder 18. The remainder of the suture 70 can be integral with the suture loop 162, or can removably attached to the suture loop 162. The suture loop 162 can be circular or oval. As another example, the suture 70 may not have the loop 162. The suture 70 can be braided. The suture 70 may not be braided.

FIG. 15B illustrates that the shuttle 14 can have one or more shuttle notches 166 or cut-outs. For example, the shuttle 14 can have two shuttle notches 166 on each lateral site of the shuttle. The shuttle notches 166 can be even longitudinally spaced and distributed along the shuttle 14. The shuttle notches 166 can be curved. The sides of the shuttle 14, other than at the notches, can be straight. The shuttle notches 166 can extend into the shuttle body 160, into the shuttle fingers 156, into the shuttle slots 158, or into any combination thereof. A radius of curvature of the shuttle notch 166 can be from about 1 mm to about 2 mm.

FIGS. 15A and 15B illustrate that the shuttle 14 can have a shuttle tip 164 on each longitudinal end. FIGS. 15A and 15B illustrate, for example, that both of the longitudinal terminal ends of the shuttle 14 can be a shuttle tip 164. The shuttle tips 164 can be blunt. The shuttle tips can be sharpened, for example, to cut tissue. The shuttle tips 164 can, for example, be beveled. FIGS. 15A and 15B illustrate that the shuttle 14 can have a slot 174. The slot 174 can be, for example, a shuttle longitudinal slot. FIGS. 15A and 15B illustrate that the suture 70 can be tied or adhered directly to suture holder 18. The suture 70 can have a suture loop 162. The suture loop 162 can circumscribe the suture holder 18. The suture 70 can be attached to the shuttle 14, for example, to the suture holder 18. Accordingly, the suture 70 can be attached to the shuttle 14 and can follow the movement of the shuttle 14. Similarly, the suture 70 can be attached to and detached from the shuttle 14, for example, attached before and detached after the desired stitching or suturing is complete.

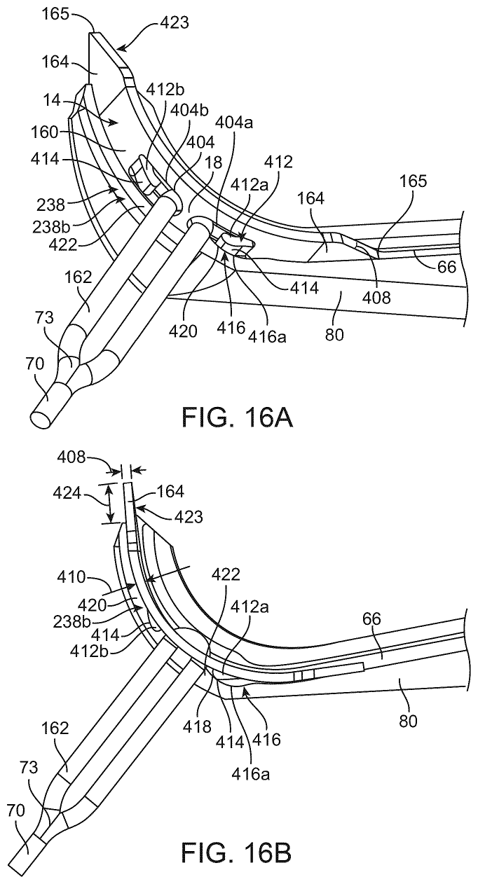

FIG. 16A illustrates that the suture holder 18 can be attached to or integrated with the shuttle 14. For example, the suture holder 18 can be a bridge integrated with the shuttle body 160. A portion of the shuttle body 160 can define the suture holder 18. As another example, the suture holder 18 can be removably attached to the shuttle 14. The suture holder 18 can be in the plane of the shuttle body 160, extend away from the plane of the shuttle body 160, or both. For example, FIG. 16A illustrates that the suture holder 18 may not extend away from the plane of the shuttle body 160. The suture loop 162 can extend around and completely or partially circumscribe the suture holder 18. The remainder of the suture 70 can be integral with the suture loop 162, or can removably attached to the suture loop 162. The suture loop 162 can be attached to or integrated with the suture 70 at a suture junction 73. The suture junction 73 can be a knot, a braid, or both. The suture loop 162 can be circular, oval, or stadium-shaped.

The shuttle 14 can have zero, one, or multiple suture holes 404, for example, 0 to 4 or more suture holes 404, including every 1 suture hole increment within this range. For example, FIG. 16A illustrates that the shuttle 14 can have a first suture hole 404a and a second suture hole 404b. The suture 70 can extend through the suture holes 404.

The shuttle tips 164 can be beveled, non-beveled, or both. For example, FIG. 16A illustrates that the shuttle tips 164 can be non-beveled.

FIG. 16A illustrates that the shuttle tips 164 can be tapered to form a terminal tip 165. The shuttle tips 164 can pierce or cut tissue with or without having a beveled surface.

FIG. 16A illustrates that the device 188 can have one or multiple male stops 412 (also referred to as male catches, male detents, stops, catchers, detents) and one or multiple female stops 416 (also referred to as female catches, female detents, stops, catchers, detents). The device 188 can have, for example, 1-10 or more male stops 412, including every 1 male stop increment within this range (e.g., 1, 2, 3, 4 or more male stops). The device 188 can have, for example, 1-10 or more female stops 416, including every 1 female stop increment within this range (e.g., 1, 2, 3, 4 or more female stops).

The male stops 412 can be attached to or integrated with the device 188. For example, the male stops 412 can be part of, attached to, or integrated with the shuttle 14. As another example, the male stops 412 can be part of, attached to, or integrated with the jaws 801. As yet another example, the device 188 can have some male stops 412 that are part of, attached to, or integrated with the shuttle 14 and can have some male stops 412 that are part of, attached to, or integrated with the jaws 801. As yet still another example, the male stops 412 can be part of, attached to, or integrated with the pushers (e.g., the first and second pushers 76, 86).

The female stops 416 can be attached to or integrated with the device 188. For example, the female stops 416 can be part of, attached to, or integrated with the shuttle 14. As another example, the female stops 416 can be part of, attached to, or integrated with the jaws 801. As yet another example, the device 188 can have some female stops 416 that are part of, attached to, or integrated with the shuttle 14 and can have some female stops 416 that are part of, attached to, or integrated with the jaws 801. As yet still another example, the female stops 416 can be part of, attached to, or integrated with the pushers (e.g., the first and second pushers 76, 86).

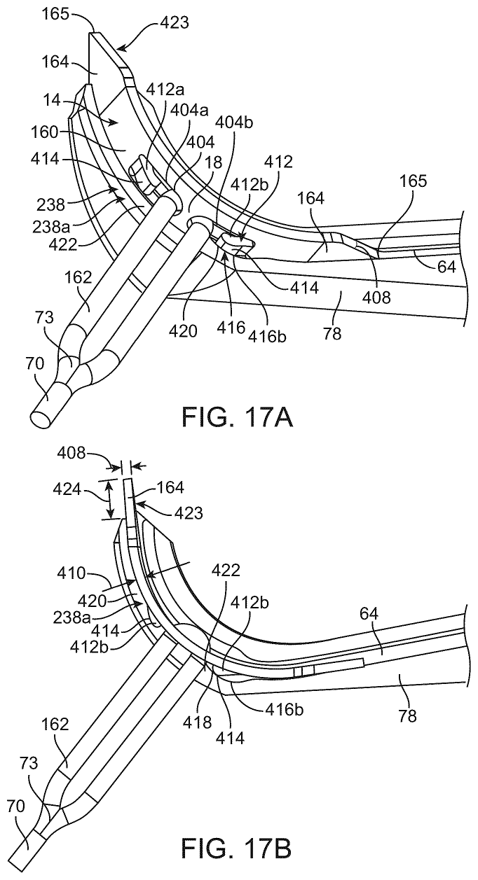

FIGS. 16A-17B illustrate, for example, that the shuttle 14 can have the male stops 412 and that the jaws 801 can have the female stops 416. For example, FIGS. 16A-17B illustrate that the shuttle 14 can have a first male stop 412a and a second male stop 412b, that the second jaw 80 can have a first female stop 416a configured to releasably engage with or releasably attach to the first male stop 412a, and that the first jaw 78 can have a second female stop 416b configured to releasably engage with or releasably attach to the second male stop 412b. As another example, the shuttle 14 can have the first male stop 412a and the second male stop 412b, that the first jaw 78 can have the first female stop 416a configured to releasably engage with or releasably attach to the first male stop 412a, and the second jaw 80 can have the second female stop 416b configured to releasably engage with or releasably attach to the second male stop 412b. Half of the first and second jaws 78, 80 are shown transparent in FIGS. 16A-17B so that the shuttle 14 can be more easily seen in the jaw tracks (e.g., the first and second tracks 64, 66), and so that the male and female stops 412, 416 can be more easily seen.

Each male stop 412 can releasably fit into, attach to, or engage with a corresponding female stop 416, for example, via a friction fit, snap fit, magnetic fit, ratchet fit, or any combination thereof. For example, the first male stop 412a can be releasably attachable to the first female stop 416a and the second male stop 412b can be releasably attachable to the second female stop 416b. When two stops (e.g., male and female stops 412, 416) are releasably attached to one another, a threshold release force can be required to release the stops from one another. The threshold release force can be from about 1.0 Newton to about 10.0 Newtons or more, including every 0.5 Newton increment within this range (e.g., 4.0 Newtons, 4.5 Newtons, 5.0 Newtons). As another example, the release force can be from about 0.5 lbs to about 1.5 lbs, including every 0.1 lb increment within this range (e.g., 1.0 lb).

The male stops 412 can be a positive feature such as a protrusion, bump, ridge, arm, extension, flexure, detent flexure, or any combination thereof. The male stops 412 can be straight and/or curved. The male stops 412 can be flexible, rigid, or both (e.g., a first portion can be flexible and a second portion can be rigid). The male stops 412 can be one or more springs. female stops 416 can be a negative feature such as a void, space, pocket, notch, hole, through hole, recess, detent recess, or any combination thereof. The female stops 416 can be flexible, rigid, or both (e.g., a first portion can be flexible and a second portion can be rigid). The male and female stops 412, 416 can include magnets that attract one another to keep the male and female stops releasably attached together.

The male stops 412 can have a male surface 414 and the female stops 416 can have a female surface 418. The male and female surfaces 414, 418 can be configured to engage with one another, slidably engage with one another, contact one another other, or any combination thereof. The female stops 416 can have a lip 420 configured to engage with, slidably engage with, or contact the male stop 412, or any combination thereof. The male stops 412 can be configured to engage with, slidably engage with, or contact the lip 420, or any combination thereof.

For each male-female stop pair (e.g., the first male and female stops 412a, 416a, and the second male and female stops 412b, 416b), the male and female surfaces 414,418 can engage with one another and/or the male stop 412 can engage with the lip 420, for example, when the male stop 412 is being forced into the female stop 416, when the male stop 412 is being withdrawn from the female stop 416, when the female stop 416 is being forced over or onto the male stop 412, when the female stop 416 is being withdrawn from the male stop 412, when the male and female stops 412, 416 are engaged with one another (also referred to as the stopped position, caught position, fixed position), or any combination thereof.

The male stops 412 can move relative to the female stops 416, vice versa, or both. The female stops 416 can move relative to the male stops 412, vice versa, or both. For example, FIGS. 16A and 16B illustrate that the male stops 412 can be translatable (e.g., slidably translatable) in the track 66, for example, in a first direction toward the first female stop 416a and in a second direction away from the first female stop 416a, or vice versa such that the first female stop 416a is translatable toward and away from a male stop 412 (e.g., for arrangements where the female stop 416 is integrated with or attached to the shuttle 14 and the male stop 412 is integrated with or attached to the second jaw 80). The first and second directions can be opposite from one another. As another example, FIGS. 17A and 17B illustrate that the male stops 412 can be translatable (e.g., slidably translatable) in the track 64, for example, in a first direction toward the second female stop 416b and in a second direction away from the second female stop 416b, or vice versa such that the second female stop 416b is translatable toward and away from a male stop 412 (e.g., for arrangements where the female stop 416 is integrated with or attached to the shuttle 14 and the male stop 412 is integrated with or attached to the first jaw 78). The first and second directions can be opposite from one another.

FIGS. 16A and 16B illustrate that when the shuttle 14 is being translated (e.g., pushed by the first pusher 76, pulled by the second pusher 86, or both) in a first direction in the second track 66 toward the first female stop 416a, the lip 420 can exert a force against the first male stop 412a that causes the first male stop 412a to flex toward a longitudinal axis of the shuttle 14. This flexure can allow the first male stop 412a to fit into the first female stop 416a. Upon passing by the lip 420 in the first direction, the first male stop 412a can rebound to its neutral position or to a less flexed position and releasably lock the shuttle 14 to the second jaw 80 via the releasable engagement between the first male stop 412a and the first female stop 416a. FIGS. 16A and 16B illustrate that when the shuttle 14 is being translated (e.g., pushed by the second pusher 86, pulled by the first pusher 76, or both) in a second direction (e.g., opposite the first direction) in the second track 66 away from the first female stop 416a, the female surface 418 can exert a force against the first male stop 412a that causes the first male stop 412a to flex toward a longitudinal axis of the shuttle 14. This flexure can allow the first male stop 412a to slide under and past the lip 420. Upon passing by the lip 420 in the second direction, the first male stop 412a can rebound to its neutral position (also referred to as a non-flexed position). When the first male stop 412a flexes, it can deflect into the first suture hole 404a. For example, the first female stop 416a can push the first male stop 412a toward and/or into the first suture hole 404a.

FIGS. 17A and 17B illustrate that when the shuttle 14 is being translated (e.g., pushed by the second pusher 86, pulled by the first pusher 76, or both) in a first direction in the first track 64 toward the second female stop 416b, the lip 420 can exert a force against the second male stop 412b that causes the second male stop 412b to flex toward a longitudinal axis of the shuttle 14. This flexure can allow the second male stop 412b to fit into the second female stop 416b. Upon passing by the lip 420 in the first direction, the second male stop 412b can rebound to its neutral position or to a less flexed position and releasably lock the shuttle 14 to the first jaw 78 via the releasable attachment between the second male stop 412b and the second female stop 416b. FIGS. 17A and 17B illustrate that when the shuttle 14 is being translated (e.g., pushed by the first pusher 76, pulled by the second pusher 86, or both) in a second direction (e.g., opposite the first direction) in the first track 64 away from the second female stop 416b, the female surface 418 can exert a force against the second male stop 412b that causes the second male stop 412b to flex toward a longitudinal axis of the shuttle 14. This flexure can allow the second male stop 412b to slide under and past the lip 420. Upon passing by the lip 420 in the second direction, the second male stop 412b can rebound to its neutral position (also referred to as a non-flexed position). When the second male stop 412b flexes, it can deflect into the second suture hole 404b. For example, the second female stop 416b can push the second male stop 412b toward and/or into the second suture hole 404b.

The lip 420 can resist passage of the first and second male stops 412a, 412b along the first and second directions into and out of the first and second female stops 416a, 416b with the threshold release force. The female surface 418 can be an inner surface of the lip 420. The lip 420 can resist passage of the first and second male stops 412a, 412b along the first direction into the first and second female stops 416a, 416b with the threshold release force or a lesser force (e.g., a force 10% to 75% of the threshold release force) than along the second direction.

The device 188 can have zero, one, or multiple male stops 412 and zero, one, or multiple female stops 416 on the device distal end 2 (e.g., closer to the jaws than to the handle 104) and/or on the device proximal end (e.g., closer to the handle 104 than to the jaws). For example, the first jaw 78 can have one or multiple male stops 412, one or multiple female stops 416, or any combination thereof. The second jaw 80 can have one or multiple male stops 412, one or multiple female stops 416, or any combination thereof. The male and/or female stops 412 and/or 416 can be attached to or integrated with the jaw, the jaw track, or both. The shuttle 14 can have one or multiple male stops 412, one or multiple female stops 416, or any combination thereof. The male stops 412 can extend away from and/or toward a longitudinal axis of the shuttle 14. The male stops 412 can extend away from and/or toward a longitudinal axis of a jaw track (e.g., tracks 64 and 66). The female stops 416 can extend away from and/or toward a longitudinal axis of the shuttle 14. The female stops 416 can extend away from and/or toward a longitudinal axis of a jaw track (e.g., tracks 64 and 66).

For example, FIGS. 16A-17B illustrate that the shuttle 14 can have a first male stop 412a and a second male stop 412b, and that the tracks 64 and 66 can each define a female stop 416 (e.g., a first female stop 416a in the second jaw 80 and a second female stop 416b in the first jaw 78 or vice versa). The first and second jaws 78, 80 can each define one or multiple female stops 416. The first male stop 412a can releasably attach to the first female stop 416a and the second male stop 412b can releasably attach to a second female stop 416b. FIGS. 16A-17B illustrate that the first and second male stops 412a, 412b can extend away from a longitudinal axis (e.g., center longitudinal axis) of the shuttle toward a longitudinal center of the shuttle 14. The first and second male stops 412a, 412b can be the same or a different dimension away from the longitudinal center of the shuttle 14.

FIGS. 16A-17B illustrate a surface 422 of the slots 238, for example, of the first slot 238a and of the second slot 238b.

FIGS. 16B and 17B illustrate that the shuttle tips 164 can have a shuttle tip thickness 408 of about 0.05 mm to about 0.75 mm, including every 0.05 mm increment within this range (e.g., 0.15 mm, 0.20 mm, 0.25 mm). As another example, the shuttle tip thickness 408 can be from about 0.0080 in. to about 0.0090 in. (e.g., 0.0085 in.).

FIGS. 16B and 17B illustrate that the shuttle 14 can have a shuttle thickness 410 (also referred to as the shuttle thickness 14_{T}) of about 0.05 mm to about 0.75 mm, including every 0.05 mm increment within this range (e.g., 0.15 mm, 0.20 mm, 0.25 mm). The shuttle tip thickness 408 can be the same or different from the shuttle thickness 410. The shuttle tip thickness 408 can be less than, equal to, or greater than the shuttle thickness 410. For example, the shuttle tip thickness 408 can be about 0.15 mm and the shuttle thickness 410 can be about 0.25 mm, or vice versa.

The shuttle 14 can be made from a single panel of material (e.g., metal). The suture holes 404 can be cut, leaving the shuttle 14 and the male stops 412. The shuttle 14 can then be bent, which can result in the male stops 412 extending out of the plane of the shuttle body 160.

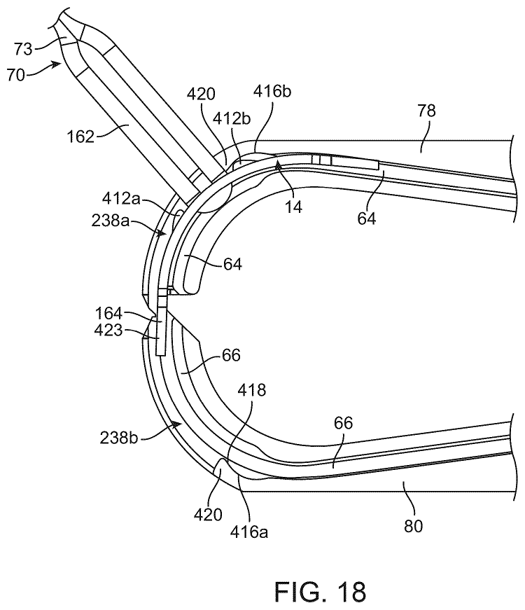

FIG. 18 illustrates that when the first and second jaws 78, 80 are closed the jaws can define a continuous track for the shuttle 14 such the tracks 64 and 66 are continuous with one another. The tracks of the first and second jaws 78, 80 can form a continuous path when the jaws 801 are in a closed configuration.

FIG. 18 illustrates that the tip of the first jaw 78 can interdigitate with the tip of the second jaw 80 when the jaws 801 are in a fully closed configuration. For example, the tip of the first jaw 78 can interdigitate with and be adjacent or in contact with the tip of the second jaw 80 when the jaws 801 are in a fully closed configuration. The jaw tips can be sharpened. The jaw tips can be tapered. The jaw tips can be sharp and seat into each other to form a continuous track when the jaws 801 are in a fully closed configuration. The jaw tips can seat into each other to connect the tracks 64 and 66 to each other. As another example, the tips of the first and second jaws 801 may not interdigitate or contact each other when the jaws 801 are in a fully closed configuration. For example, a gap (e.g., the gap 906) can be between the jaws 801 when the jaws 801 are in a fully closed configuration.

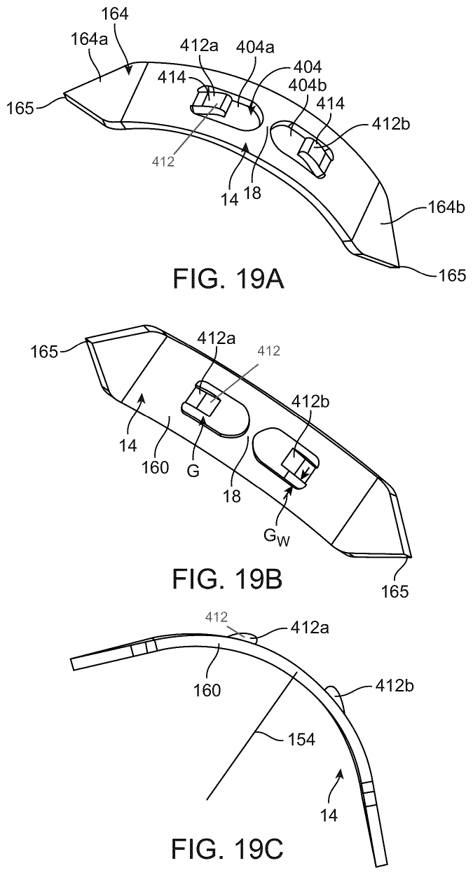

FIG. 19A illustrates the shuttle 14 in FIGS. 16A-18. FIG. 19A illustrates that the shuttle 14 can have a shuttle first tip 164a and a shuttle second tip 164b.

FIG. 19B illustrates that there can be a gap G on each side of the male stop 412 between the male stop 412 and the shuttle body 160. The gap G can advantageously inhibit or prevent pressure from forming in the jaws by allowing gas, liquid, or solids to flow or pass through the gap G as the shuttle 14 is advanced into the jaws. There can be a gap on each lateral side of the male stop 412 as shown in FIG. 19B. As another example, there may not be any gaps G between the male stops 412 and the shuttle body 160, or the gap G can be on only one side of the male stop 412 instead of both sides as shown in FIG. 19B. The gaps G can have a gap width G_{W} that can be, for example, constant (e.g., as shown in FIG. 19B) or tapered. As another example, the gaps G can have multiple gap widths G_{W}. For example, FIG. 19B illustrates that the gap G can have a constant width, for example, as measured between the lateral edge or surface of the male stop 412 and the lateral edge or surface of the shuttle body 160.

FIG. 19B illustrates that the terminal tips 165 can have a sharpened edge. The sharpened edge can be configured to pierce tissue.

FIG. 19C illustrates that the male stops 412 (e.g., first and second male stops 412a, 412b) can extend away from a longitudinal axis of the shuttle 14 out of the plane of the shuttle body 160.

The shuttle longitudinal axis (e.g., longitudinal axis 157) can be flat or curved.

FIG. 19C illustrates that the shuttle radius of curvature 154 can be from about 2.00 mm to about 5.00 mm or more, including every 0.01 mm increment within this range (e.g., 2.84 mm).

The shuttle 14 can be straight or have a preformed bend or curve (e.g., having the radius of curvature 154). The shuttle 14 can have a preformed bend having a radius of curvature of about 40% to about 200% of the radius of curvature 154, including every 1% increment within this range (e.g., 50%).

The curvature of the shuttle 14 can be constant. The curvature of the shuttle 14 can be fixed. The shuttle 14 can be flexible. The shuttle 14 can be rigid. The shuttle 14 can transition between curved and straight configurations. Having a preformed bend within this range can reduce the strain on the shuttle 14. For example, the strain can be reduced for variations where the shuttle 14 shifts between straight and curved configurations when moving within and/or between the jaws.

Each male stop 412 can be a radial male stop 412_{R} and/or a lateral male stop 412_{L}. The shuttle 14 can have radial male stops 412_{R} and/or lateral male stops 412_{L}. FIGS. 19A-19C illustrate, for example, that the male stops 412 can be radial male stops. Radial male stops 412_{R} can extend radially away from the shuttle 14. Radial male stops 412_{R} can, for example, extend radially away from a shuttle longitudinal axis 14_{A1}. Lateral male stops 412_{L} can extend laterally away from the shuttle 14. Lateral male stops 412_{L} can, for example, extend laterally away from the shuttle longitudinal axis 14_{A1}.

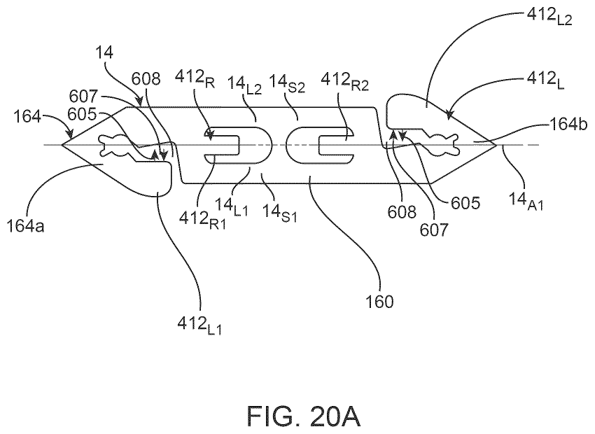

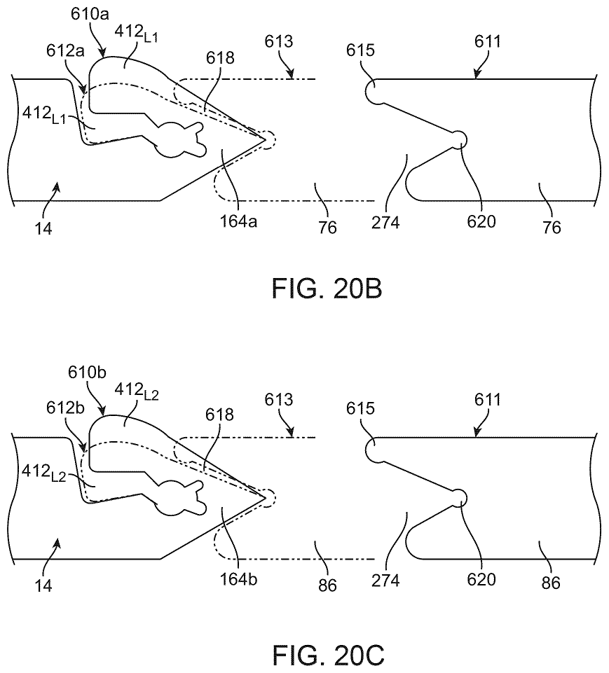

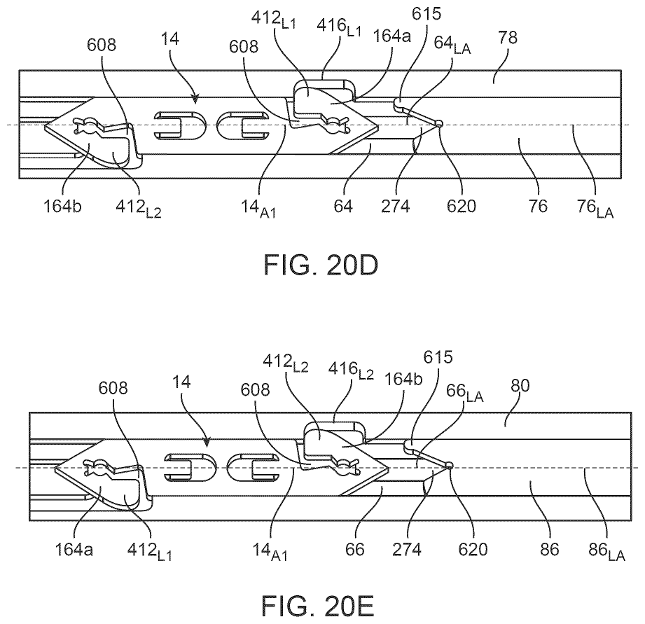

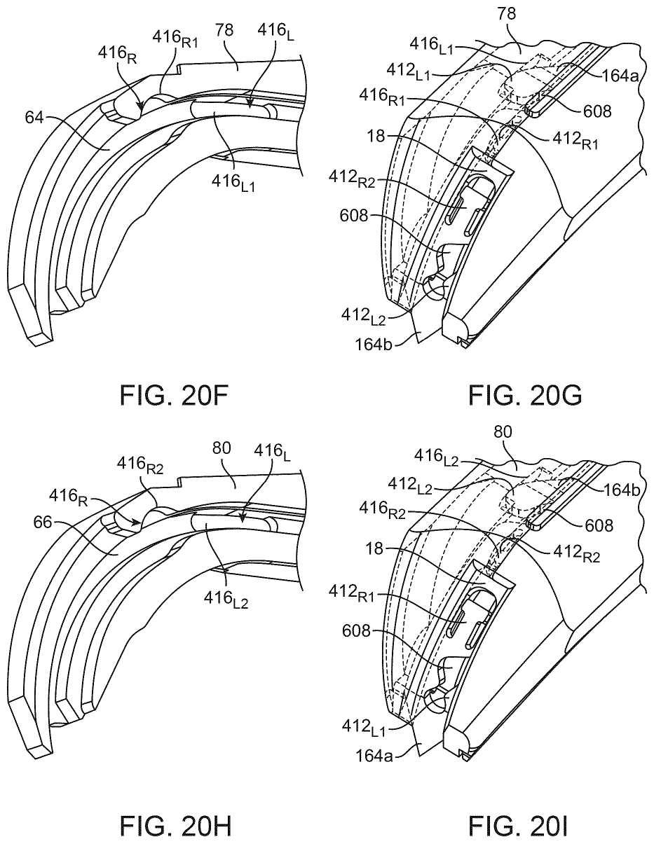

FIG. 20A shows, for example, that the shuttle 14 can have radial male stops 412_{R} and/or lateral male stops 412_{L}. The shuttle 14 can have, for example, 0-8 radial male stops 412_{R}, including every 1 radial male stop increment within this range (e.g., 0 radial male stops, 1 radial male stop, 2 radial male stops). The shuttle 14 can have, for example, 0-8 lateral male stops 412_{L}, including every 1 lateral male stop increment within this range (e.g., 0 lateral male stops, 1 lateral male stop, 2 lateral male stops). For example, FIG. 20A illustrates that the shuttle 14 can have a first radial male stop 412_{R1}, a second radial male stop 412_{R2}, a first lateral male stop 412_{L1}, and a second lateral male stop 412_{L2}, or any combination thereof (e.g., only the first and second lateral male stops 412_{L1}, 412_{L2}).

FIG. 20A illustrates that the shuttle 14 can have a shuttle first longitudinal end 14_{S1}, a shuttle second longitudinal end 14_{S2}, a shuttle first lateral side 14_{L1}, and a shuttle second lateral side 14_{L2}. The lateral male stops 412_{L} can be anywhere along the length of the shuttle 14 and can be on one or both lateral sides of the shuttle 14. For example, FIG. 20A illustrates that the shuttle first longitudinal end 14_{S1} and the shuttle first lateral side 14_{L1} can have a lateral male stop (e.g., the first lateral male stop 412_{L1}) and that the shuttle second longitudinal end 14_{S2} and the shuttle second lateral side 14_{L2} can have a lateral male stop (e.g., the second lateral male stop 412_{L2}) in the arrangement shown.

The lateral male stops 412_{L} can be, for example, springs moveable (e.g., deflectable) toward and away from the shuttle longitudinal axis 14_{A1} as shown by arrows 605 and 607 when engaging and/or disengaging with a female stop 416. As shown in FIG. 20A, for example, the first lateral male stop 412_{L1} can be moveable toward (e.g., arrow 605) the shuttle longitudinal axis 14_{A1} when exiting a female stop 416 and can be moveable away from (e.g., arrow 607) the shuttle longitudinal axis 14_{A1} when entering a female stop 416. The first lateral male stop 412_{L1} can be moveable toward (e.g., arrow 605) the shuttle second lateral side 14_{L2} when exiting a female stop 416 and can be moveable away from (e.g., arrow 607) the shuttle second lateral side 14_{L2} when entering a female stop 416. As further shown in FIG. 20A, the second lateral male stop 412_{L2} can be moveable toward (e.g., arrow 605) the shuttle longitudinal axis 14_{A1} when exiting a female stop 416 and can be moveable away from (e.g., arrow 607) the shuttle longitudinal axis 14_{A1} when entering a female stop 416. The second lateral male stop 412_{L2} can be moveable toward (e.g., arrow 605) the shuttle first lateral side 14_{L1} when exiting a female stop 416 and can be moveable away from (e.g., arrow 607) the shuttle first lateral side 14_{L1} when entering a female stop 416.

FIG. 20A illustrates that the lateral male stops 412_{L} can form part of the shuttle tips 164 (e.g., first and second tips 164a, 164b) and/or can form part of the shuttle body 160.

The lateral male stops 412_{L} can be moveable (e.g., deflectable) between multiple configurations. For example, FIG. 20A illustrates that the lateral male stops 412_{L} (e.g., the first and second lateral male stops 412_{L1}, 412_{L2}) can be moveable (e.g., deflectable) back and forth between a male stop first configuration (e.g., the configurations shown in FIG. 20A) and a male stop second configuration (e.g., see FIGS. 20B and 20C).

The male stop first configuration can be a neutral configuration or a non-deflected configuration of the lateral male stops 412_{L}. The lateral male stops 412_{L} can have the male stop first configuration, for example, when engaged with a female stop 416.

The male stop second configuration can be a deflected configuration of the lateral male stops 412_{L}. The male stop second configuration can be, for example, a deflected configuration. The male stop second configuration can be, for example, a deflected configuration in which the lateral male stop 412_{L} has been moved (e.g., deflected) in direction 605 away from the male stop first configuration. The lateral male stops 412_{L} can have the male stop second configuration, for example, when the lateral male stop 412 is in a shuttle track (e.g., tracks 64 and/or 66) between the tip of a jaw and a female stop 416, when the lateral male stop 412_{L} is partially engaged with a female stop 416, or both.

FIG. 20A illustrates that the shuttle 14 can have a space 608 when the lateral male stops 412_{L} are in the male stop first configuration. The space 608 can be, for example, between the lateral male stops 412_{L} and the shuttle body 160. The space 608 can be smaller when the lateral male stops 412_{L} are in the male stop second configuration than when in the male stop first configuration. For example, the space 608 can be partially or completely closed when the lateral male stops 412_{L} are in the male stop second configuration than when in the male stop first configuration. When the lateral male stops 412_{L} are moved from the male stop first configuration to the male stop second configuration, the lateral male stops 412_{L} can move into the space 608. When the lateral male stops 412_{L} move into the space 608, the lateral male stops 412_{L} can pivot about the shuttle tips 164 (e.g., about shuttle first and second tips 164a, 164b).

FIG. 20A illustrates the shuttle 14 and the male stops 412 (e.g., the radial and lateral male stops 412_{R}, 412_{L}) in a flat configuration before being shape-set into a curved configuration.

FIG. 20B illustrates two configurations of the first pusher 76 and the first lateral male stop 412_{L1}, for example, a first configuration in which the first pusher 76 is not engaged with the first lateral male stop 412_{L1}, and a second configuration in which the first pusher 76 is engaged with the first lateral male stop 412_{L1}. When the first pusher 76 is not engaged with the first lateral male stop 412_{L1} and the first lateral stop 412_{L1} is engaged with a female stop 416, the shuttle 14 (e.g., the first lateral male stop 412_{L1}) can have the features and arrangement shown by the solid lines in FIG. 20B (e.g., as shown by arrow 610a) and the first pusher 76 can have the features and arrangement shown by the solid lines in FIG. 20B (e.g., as shown by arrow 611). When the first pusher 76 is engaged with the first lateral male stop 412_{L1} and the first pusher 76 has disengaged the first lateral stop 412_{L1} from a female stop 416, the shuttle 14 (e.g., the first lateral male stop 412_{L1}) can have the features and arrangement shown by the dashed lines in FIG. 20B (as shown by arrow 612a) and the first pusher 76 can have the features and arrangement shown by the dashed lines in FIG. 20B (as shown by arrow 613). The configuration of the first lateral male stop 412_{L1} shown by arrow 610a is also referred to as the configuration 610a, where the configuration 610a can be, for example, the male stop first configuration of the first lateral male stop 412_{L1}. The configuration of the first lateral male stop 412_{L1} shown by arrow 612a is also referred to as the configuration 612a, where the configuration 612a can be, for example, the male stop second configuration of the first lateral male stop 412_{L1}. The configuration 610a can be, for example, a non-deflected configuration of the first lateral male stop 412_{L1}. The configuration 612a can be, for example, a fully deflected configuration of the first lateral male stop 412_{L1}.

FIG. 20B illustrates that when the first pusher 76 is moved into contact with the second lateral stop 412_{L2}, the second lateral stop 412_{L2} can be moved from the first configuration as shown by arrow 610a to the second configuration as shown by arrow 612a. The arrow 610a shows, for example, the male stop first configuration of the second lateral stop 412_{L2}, and the arrow 612a shows, for example, the male stop second configuration of the second lateral stop 412_{L2}. FIG. 20B illustrates that the first pusher 76 can have a lateral stop engager 615. When the first pusher 76 is moved into contact with the second lateral stop 412_{L2}, the lateral stop engager 615 can move the second lateral stop 412_{L2} from the male stop first configuration (e.g., the configuration shown by arrow 610a) to the male stop second configuration (e.g., the configuration shown by arrow 612a). The lateral stop engager 615 can advantageously inhibit or prevent the shuttle 14 and the first pusher 76 from becoming stuck together by creating a space 618 between the shuttle first tip 164a and the first pusher 76 when the shuttle first tip 164a is in the shuttle seat 274 of the first pusher 76. As another example, the first pusher 76 may not have the lateral stop engager 615 such that there may not be the space 618 when the shuttle first tip 164a is in the shuttle seat 274 of the first pusher 76. FIG. 20B illustrates that the first pusher 76 can have a tip space 620 that inhibits or prevents the terminal tip 165 of the shuttle first tip 164a from contacting the first pusher 76 to inhibit or prevent the first pusher 76 from dulling or chipping the shuttle first tip 164a (e.g., the terminal tip 165).

FIG. 20C illustrates two configurations of the second pusher 86 and the second lateral male stop 412_{L2}, for example, a first configuration in which the second pusher 86 is not engaged with the second lateral male stop 412_{L2}, and a second configuration in which the second pusher 86 is engaged with the second lateral male stop 412_{L2}. When the second pusher 86 is not engaged with the second lateral male stop 412_{L2} and the second lateral stop 412_{L2} is engaged with a female stop 416, the shuttle 14 (e.g., the second lateral male stop 412_{L2}) can have the features and arrangement shown by the solid lines in FIG. 20C (e.g., as shown by arrow 610b) and the second pusher 86 can have the features and arrangement shown by the solid lines in FIG. 20C (e.g., as shown by arrow 611). When the second pusher 86 is engaged with the second lateral male stop 412_{L2} and the second pusher 86 has disengaged the second lateral stop 412_{L2} from a female stop 416, the shuttle 14 (e.g., the second lateral male stop 412_{L2}) can have the features and arrangement shown by the dashed lines in FIG. 20C (as shown by arrow 612b) and the second pusher 86 can have the features and arrangement shown by the dashed lines in FIG. 20C (as shown by arrow 613). The configuration of the second lateral male stop 412_{L2} shown by arrow 610b is also referred to as the configuration 610b, where the configuration 610b can be, for example, the male stop first configuration of the second lateral male stop 412_{L2}. The configuration of the second lateral male stop 412_{L2} shown by arrow 612b is also referred to as the configuration 612b, where the configuration 612b can be, for example, the male stop second configuration of the second lateral male stop 412_{L2}. The configuration 610b can be, for example, a non-deflected configuration of the second lateral male stop 412_{L2}. The configuration 612b can be, for example, a fully deflected configuration of the second lateral male stop 412_{L2}.

FIG. 20C illustrates that when the second pusher 86 is moved into contact with the second lateral stop 412_{L2}, the second lateral stop 412_{L2} can be moved from the first configuration as shown by arrow 610b to the second configuration as shown by arrow 612b. The arrow 610b shows, for example, the male stop first configuration of the second lateral stop 412_{L2}, and the arrow 612b shows, for example, the male stop second configuration of the second lateral stop 412_{L2}. FIG. 20C illustrates that the second pusher 86 can have a lateral stop engager 615. When the second pusher 86 is moved into contact with the second lateral stop 412_{L2}, the lateral stop engager 615 can move the second lateral stop 412_{L2} from the male stop first configuration (e.g., the configuration shown by arrow 610b) to the male stop second configuration (e.g., the configuration shown by arrow 612b). The lateral stop engager 615 can advantageously inhibit or prevent the shuttle 14 and the second pusher 86 from becoming stuck together by creating a space 618 between the shuttle second tip 164b and the second pusher 86 when the shuttle second tip 164b is in the shuttle seat 274 of the second pusher 86. As another example, the second pusher 86 may not have the lateral stop engager 615 such that there may not be the space 618 when the shuttle second tip 164b is in the shuttle seat 274 of the second pusher 86. FIG. 20C illustrates that the second pusher 86 can have a tip space 620 that inhibits or prevents the terminal tip 165 of the shuttle second tip 164b from contacting the second pusher 86 to inhibit or prevent the second pusher 86 from dulling or chipping the shuttle second tip 164b (e.g., the terminal tip 165).

FIGS. 20D and 20E illustrate that first and second jaws 78, 80 can each have one or multiple lateral female stops 416_{L} that can, for example, extend laterally away from the tracks 64 and 66. The first jaw 78 can have, for example, 0-8 lateral female stops 416_{L}, including every 1 lateral female stop increment within this range (e.g., 0 lateral female stops, 1 lateral female stop, 2 lateral female stops). The second jaw 80 can have, for example, 0-8 lateral female stops 416_{L}, including every 1 lateral female stop increment within this range (e.g., 0 lateral female stops, 1lateral female stop, 2 lateral female stops). For example, FIGS. 20D and 20E illustrate that the first jaw 78 can have a first lateral female stop 416_{L1} and that the second jaw 80 can have a second lateral female stop 416_{L2}. FIG. 20D illustrates that the first lateral female stop 416_{L1} can extend laterally away from the track 64 and FIG. 20E illustrates that the second lateral female stop 416_{L2} can extend laterally away from the track 66. For example, the first lateral female stop 416_{L1} can extend laterally away from the longitudinal axis (e.g., a center longitudinal axis 64_{LA}) of the track 64, and the second lateral female stop 416_{L2} can extend laterally away from the longitudinal axis (e.g., a center longitudinal axis 66_{LA}) of the track 64.

FIG. 20D illustrates that when the first lateral male stop 412_{L1} is engaged with the first lateral female stop 416_{L1}, the first lateral male stop 412_{L1} can have the male stop first configuration (e.g., a partially deflected configuration or a non-deflected configuration such as, for example, the configuration 610a). FIG. 20D illustrates that when the first lateral male stop 412_{L1} is engaged with the first lateral female stop 416_{L1}, the second lateral male stop 412_{L2} can have the male stop first configuration (e.g., a partially deflected configuration or a non-deflected configuration such as, for example, the configuration 610b). When the first lateral male stop 412_{L1} is engaged with the first lateral female stop 416_{L1}, the male stop first configuration of the second lateral male stop 412_{L2} can be the same as the male stop first configuration of the first lateral male stop 412_{L1} (e.g., as shown in FIG. 20D). The configuration 610a can be, for example, the same as the configuration 610b. As another example, when the first lateral male stop 412_{L1} is engaged with the first lateral female stop 416_{L1}, the configuration of the second lateral male stop 412_{L2} can be different from the configuration of the first lateral male stop 412_{L1}. For example, when the first lateral male stop 412_{L1} is engaged with the first lateral female stop 416_{L1} as shown in FIG. 20D, the first lateral male stop 412_{L1} can have the male stop first configuration and the second lateral male stop 412_{L2} can have the male stop second configuration (e.g., the configuration 612b or a partially deflected configuration somewhere between the configurations 610b and 612b). FIG. 20D illustrates that the longitudinal axis 76_{LA} of the first pusher 76, the longitudinal axis 64_{LA} of the track 64, and the shuttle longitudinal axis 14_{A1} can be coincident with each other when the shuttle 14 is in the first jaw 78. The shuttle 14, the track 64, and the first pusher 76 are shown flat in FIG. 20D for illustrative purposes to more easily see the relationship between the features shown.

FIG. 20E illustrates that when the second lateral male stop 412_{L2} is engaged with the second lateral female stop 416_{L2}, the second lateral male stop 412_{L2} can have the male stop first configuration (e.g., a partially deflected configuration or a non-deflected configuration such as, for example, the configuration 610b). FIG. 20E illustrates that when the second lateral male stop 412_{L2} is engaged with the second lateral female stop 416_{L2}, the first lateral male stop 412_{L1} can have the male stop first configuration (e.g., a partially deflected configuration or a non-deflected configuration such as, for example, the configuration 610a). When the second lateral male stop 412_{L2} is engaged with the second lateral female stop 416_{L2}, the male stop first configuration of the first lateral male stop 412_{L1} can be the same as the male stop first configuration of the second lateral male stop 412_{L2} (e.g., as shown in FIG. 20E). The configuration 610a can be, for example, the same as the configuration 610b. As another example, when the second lateral male stop 412_{L2} is engaged with the second lateral female stop 416_{L2}, the configuration of the first lateral male stop 412_{L1} can be different from the configuration of the second lateral male stop 412_{L2}. For example, when the second lateral male stop 412_{L2} is engaged with the second lateral female stop 416_{L2} as shown in FIG. 20E, the second lateral male stop 412_{L2} can have the male stop first configuration and the first lateral male stop 412_{L1} can have the male stop second configuration (e.g., the configuration 612a or a partially deflected configuration somewhere between the configurations 610a and 612a). FIG. 20E illustrates that the longitudinal axis 86_{LA} of the second pusher 86, the longitudinal axis 66_{LA} of the track 66, and the shuttle longitudinal axis 14_{A1} can be coincident with each other when the shuttle 14 is in the second jaw 80. The shuttle 14, the track 66, and the second pusher 86 are shown flat in FIG. 20E for illustrative purposes to more easily see the relationship between the features shown.

FIGS. 20D and 20E illustrate that the lateral male stops 412_{L} can be springs biased to have the male stop first configuration. For example, when the lateral male stops 412_{L} (e.g., the first and second lateral male stops 412_{L1}, 412_{L2}) are in the male stop second configuration, the lateral male stops 412_{L} can be biased to return (e.g., passively return) to the male stop first configuration. For example, when the lateral male stops 412_{L} are in the male stop second configuration, the lateral male stops 412_{L} can be biased to rebound or spring back to the male stop first configuration. This can advantageously allow the lateral male stops 412_{L} (e.g., the first and second lateral male stops 412_{L1}, 412_{L2}) to passively engage with the lateral female stops 416_{L} (e.g., the first and second lateral female stops 416_{L1}, 416_{L2}) as lateral male stops 412_{L} are moved into the lateral female stops 416_{L}.

FIG. 20F illustrates the track 64 of FIG. 20D having a curve in the first jaw 78, with half of the first jaw 78 shown transparent for illustrative purposes.

FIG. 20F illustrates that the first lateral female stop 416_{L1} can be straight. As another example, the first lateral female stop 416_{L1} can have a curve having a radius of curvature that is the same as, for example, the radius of curvature of the shuttle 14.

FIG. 20F illustrates that the first jaw 78 can have a radial female stop 416_{R}, a lateral female stop 416_{L}, or both. For example, FIG. 20F illustrates that the first jaw 78 can have a first radial female stop 416_{R1} and the first lateral female stop 416_{L1}.

FIG. 20G illustrates the shuttle 14 in the first jaw 78 of FIG. 20F with half of the first jaw 78 shown transparent for illustrative purposes. FIG. 20G illustrates the first lateral male stop 412_{L1} engaged with the first lateral female stop 416_{L1}, and the first radial male stop 412_{R1} engaged with the first radial female stop 416_{R1}.

FIG. 20H illustrates the track 66 of FIG. 20E having a curve in the second jaw 80, with half of the second jaw 80 shown transparent for illustrative purposes.

FIG. 20H illustrates that the second lateral female stop 416_{L2} can be straight. As another example, the second lateral female stop 416_{L2} can have a curve having a radius of curvature that is the same as, for example, the radius of curvature of the shuttle 14.

FIG. 20H illustrates that the second jaw 80 can have a radial female stop 416_{R}, a lateral female stop 416_{L}, or both. For example, FIG. 20H illustrates that the second jaw 80 can have a second radial female stop 416_{R2} and the second lateral female stop 416_{L2}.

FIG. 20I illustrates the shuttle 14 in the second jaw 80 of FIG. 20H with half of the second jaw 80 shown transparent for illustrative purposes. FIG. 20I illustrates the second lateral male stop 412_{L2} engaged with the second lateral female stop 416_{L2}, and the second radial male stop 412_{R2} engaged with the second radial female stop 416_{R2}.

FIGS. 20G and 20I illustrate that the first and second radial male stops 412_{R1} can have a hammerhead shape.

The first and second jaws 78, 80 can have lateral female stops 416_{L} in addition to or in lieu of radial female stops 412_{R}.

The first and second jaws 78, 80 can have radial female stops 412_{R} in addition to or in lieu of lateral female stops 416_{L}.

The lateral female stops 416_{L} can have the same features and functions as the radial female stops 416_{R} but can extend laterally from the shuttle 14 as opposed to radially from the shuttle 14.

FIGS. 20F-20I illustrate, for example, the shuttle 14 with four male stops 412 and the first and second jaws 78, 80 each with two female stops 416. The male stops 412 can deflect toward the shuttle longitudinal axis 14_{A1} when the shuttle 14 is moved away from the female stops 416 the shuttle 14 is engaged with. The male stops 412 can deflect toward the shuttle longitudinal axis 14_{A1} when the shuttle 14 is moved out of the female stops 416 the shuttle 14 is engaged with.

The first lateral male stop 412_{L1} can deflect from the male stop first configuration to the male stop second configuration when the second pusher 86 pushes the shuttle 14 into the track 64. For example, the tip of the first jaw 78 (e.g., a wall of the first jaw 78 near the tip) can defect the first lateral male stop 412_{L1} into the male stop second configuration when the shuttle 14 enters the track 64. The first lateral male stop 412_{L1} can passively return to a less deflected configuration (e.g., to the male stop first configuration) when the first lateral male stop 412_{L1} enters the first lateral female stop 416_{L1}. The second pusher 86 can, for example, push the first lateral male stop 412_{L1} into the first lateral female stop 416_{L1}. The first lateral male stop 412_{L1} can deflect from the less deflected configuration (e.g., from the male stop first configuration) to a more deflected configuration (e.g., to the male stop second configuration) when the first lateral male stop 412_{L1} exits the first lateral female stop 416_{L1}. The first pusher 76 can, for example, push the first lateral male stop 412_{L1} out of the first lateral female stop 416_{L1}. The first pusher 76 and/or an edge or surface of the first lateral female stop 416_{L1} can cause the first lateral male stop 412_{L1} to be deflected when the first pusher 76 moves the first lateral male stop 412_{L1} out of the first lateral female stop 416_{L1} toward the track 66.

The second lateral male stop 412_{L2} can deflect from the male stop first configuration to the male stop second configuration when the first pusher 76 pushes the shuttle 14 into the track 66. For example, the tip of the second jaw 80 (e.g., a wall of the second jaw 80 near the tip) can defect the second lateral male stop 412_{L2} into the male stop second configuration when the shuttle 14 enters the track 66. The second lateral male stop 412_{L2} can passively return to a less deflected configuration (e.g., to the male stop first configuration) when the second lateral male stop 412_{L2} enters the second lateral female stop 416_{L2}. The first pusher 76 can, for example, push the second lateral male stop 412_{L2} into the second lateral female stop 416_{L2}. The second lateral male stop 412_{L2} can deflect from the less deflected configuration (e.g., from the male stop first configuration) to a more deflected configuration (e.g., to the male stop second configuration) when the second lateral male stop 412_{L2} exits the second lateral female stop 416_{L2}. The second pusher 86 can, for example, push the second lateral male stop 412_{L2} out of the second lateral female stop 416_{L2}. The second pusher 86 and/or an edge or surface of the second lateral female stop 416_{L2} can cause the second lateral male stop 412_{L2} to be deflected when the second pusher 86 moves the second lateral male stop 412_{L2} out of the second lateral female stop 416_{L2} toward the track 64.

When the second pusher 86 is advanced, the second pusher 86 can push the shuttle 14 from the track 66 to the track 64 toward the first lateral female stop 416_{L1} until the first lateral male stop 412_{L1} enters the first lateral female stop 416_{L1}. The second pusher 86 can push the first lateral male stop 412_{L1} into the first lateral female stop 416_{L1}. When the first lateral male stop 412_{L1} is engaged with the first lateral female stop 416_{L1}, the shuttle 14 can be passively retained in the first jaw 78 until the first pusher 76 pushes the first lateral male stop 412_{L1} out of the first lateral female stop 416_{L1}. When the first pusher 76 is advanced, the first pusher 76 can push the first lateral male stop 412_{L1} out of the first lateral female stop 416_{L1} such that the first pusher 76 can push the shuttle 14 from the track 64 to the track 66 toward the second lateral female stop 416_{L2} until the second lateral male stop 412_{L2} enters the second lateral female stop 416_{L2}.

When the first pusher 76 is advanced, the first pusher 76 can push the shuttle 14 from the track 64 to the track 66 toward the second lateral female stop 416_{L2} until the second lateral male stop 412_{L2} enters the second lateral female stop 416_{L2}. The first pusher 76 can push the second lateral male stop 412_{L2} into the second lateral female stop 416_{L2}. When the second lateral male stop 412_{L2} is engaged with the second lateral female stop 416_{L2}, the shuttle 14 can be passively retained in the second jaw 80 until the second pusher 86 pushes the second lateral male stop 412_{L2} out of the second lateral female stop 416_{L2}. When the second pusher 86 is advanced, the second pusher 86 can push the second lateral male stop 412_{L2} out of the second lateral female stop 416_{L2} such that the second pusher 86 can push the shuttle 14 from the track 66 to the track 64 toward the first lateral female stop 416_{L1} until the first lateral male stop 412_{L1} enters the first lateral female stop 416_{L1}.

FIGS. 20G and 20I illustrate that the male stops 412 can be passively retained in the female stops 416. FIGS. 20G and 20I illustrate that when the first lateral male stop 412_{L1} is engaged with the first lateral female stop 416_{L1}, the shuttle 14 can be passively retained in the first jaw 78, and that when the second lateral male stop 412_{L2} is engaged with the second lateral female stop 416_{L2}, the shuttle 14 can be passively retained in the second jaw 80. Movement of the shuttle 14, for example, via the first and second pushers 76, 86, can engage and disengage the male and female stops 412, 416 from each other. For example, for engagement, the first pusher 76 can move the second lateral male stop 412_{L2} into the second lateral female stop 416_{L2} to engage the second lateral male stop 412_{L2} with the second lateral female stop 416_{L2}, and the second pusher 86 can move the first lateral male stop 412_{L1} into the first lateral female stop 416_{L1} to engage the first lateral male stop 412_{L1} with the first lateral female stop 416_{L1}. As another example, for disengagement, the first pusher 76 can move the first lateral male stop 412_{L1} out of the first lateral female stop 416_{L1} to disengage the first lateral male stop 412_{L1} from the first lateral female stop 416_{L1}, and the second pusher 86 can move the second lateral male stop 412_{L2} out of the second lateral female stop 416_{L2} to disengage the second lateral male stop 412_{L2} from the second lateral female stop 416_{L2}. The male stops 412 and the female stops 416 can thereby be passive stops, where movement of the shuttle 14 can cause the male stops 412 to engage with and disengage from the female stops 416.

When the first pusher 76 moves the shuttle 14 (e.g., the second lateral male stop 412_{L2}) into engagement with the second jaw 80 (e.g., the second lateral female stop 416_{L2}), the second lateral male stop 412_{L2} can be deflected from the male stop first configuration to the male stop second configuration, for example, by the tip of the second jaw 80, and the second lateral male stop 412_{L2} can rebound from the male stop second configuration back to the male stop first configuration, for example, as the second lateral male stop 412_{L2} is moved into the second lateral female stop 416_{L2}.

When the second pusher 86 moves the shuttle 14 (e.g., the second lateral male stop 412_{L2}) out of engagement with the second jaw 80 (e.g., the second lateral female stop 416_{L2}), the second lateral male stop 412_{L2} can be deflected from the male stop first configuration to the male stop second configuration, for example, by an edge or surface of the track 66 and/or by an edge or surface of the second lateral female stop 416_{L2} as the second pusher 86 moves the shuttle 14 toward the first jaw 78. The second lateral male stop 412_{L2} can remain in the male stop second configuration until the second lateral male stop 412_{L2} exits the second jaw 80 or until the second lateral male stop 412_{L2} reenters the second lateral female stop 416_{L2}, at which point the second lateral male stop 412_{L2} can rebound back to the male stop first configuration.

When the second pusher 86 moves the shuttle 14 (e.g., the first lateral male stop 412_{L1}) into engagement with the first jaw 78 (e.g., the first lateral female stop 416_{L1}), the first lateral male stop 412_{L1} can be deflected from the male stop first configuration to the male stop second configuration, for example, by the tip of the first jaw 78, and the first lateral male stop 412_{L1} can rebound from the male stop second configuration back to the male stop first configuration, for example, as the first lateral male stop 412_{L1} is moved into the first lateral female stop 416_{L1}.

When the first pusher 76 moves the shuttle 14 (e.g., the first lateral male stop 412_{L1}) out of engagement with the first jaw 78 (e.g., the first lateral female stop 416_{L1}), the first lateral male stop 412_{L1} can be deflected from the male stop first configuration to the male stop second configuration, for example, by an edge or surface of the track 64 and/or by an edge or surface of the first lateral female stop 416_{L1} as the first pusher 76 moves the shuttle 14 toward the second jaw 80. The first lateral male stop 412_{L1} can remain in the male stop second configuration until the first lateral male stop 412_{L1} exits the first jaw 78 or until the first lateral male stop 412_{L1} reenters the first lateral female stop 416_{L1}, at which point the first lateral male stop 412_{L1} can rebound back to the male stop first configuration.

FIGS. 20F and 20G illustrate that the first radial male stop 412_{R1} and the first lateral male stop 412_{L1} can engage with and disengage from the first radial female stop 416_{R1} and the first lateral female stop 416_{L1}, respectively, at the same time.

FIGS. 20H and 20I illustrate that the second radial male stop 412_{R2} and the second lateral male stop 412_{L2} can engage with and disengage from the second radial female stop 416_{R2} and the second lateral female stop 416_{L2}, respectively, at the same time.

FIGS. 20A-20I illustrate that the shuttle 14 can have radial male stops and lateral male stops. As another example, the shuttle 14 in FIGS. 20A-20I may not have the radial male stops.

FIGS. 20A-20I illustrate that the jaws 801 can have radial female stops and lateral female stops. As another example, the jaws 801 in FIGS. 20A-20I may not have the radial male stops.

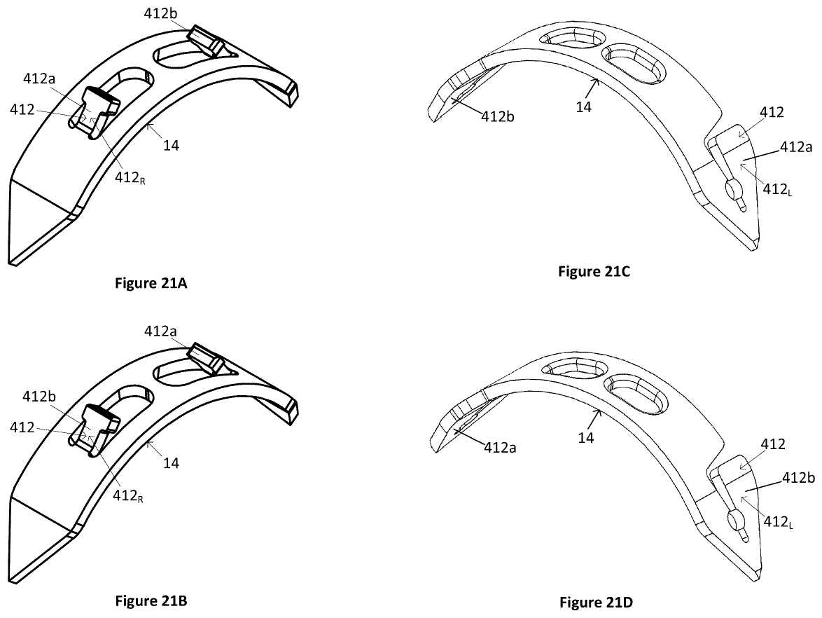

FIGS. 21A and 21B illustrate that the male stops 412 (e.g., the first and second males stops 412a, 412b) can be radial male stops 412_{R}. The first and second male stops 412a, 412b in FIGS. 21A and 21B can be, for example, first and second radial male stops 412_{R1}, 412_{R2}, respectively. FIGS. 21A and 21B illustrate that the male stops 412 (e.g., the first and second males stops 412a, 412b) can extend radially away from the shuttle 14. FIGS. 21A and 21B illustrate that the male stops 412 (e.g., the first and second males stops 412a, 412b) can have a hammerhead shape such that a first end of the males stops 412 can be smaller (e.g., narrower) than a second end of the male stops 412. FIGS. 21A and 21B illustrate that the male stops 412 (e.g., the first and second male stops 412a, 412b) can be deflectable toward and away a longitudinal axis of the shuttle 14. For example, FIGS. 21A and 21B illustrate that the male stops 412 (e.g., the first and second male stops 412a, 412b) can be deflectable toward and away from the openings in the shuttle 14 shown in FIGS. 21A and 21B.

FIGS. 21C and 21D illustrate that the male stops 412 (e.g., the first and second males stops 412a, 412b) can be lateral male stops 412_{L}. The first and second male stops 412a, 412b in FIGS. 21C and 21D can be, for example, first and second lateral male stops 412_{L1}, 412_{L2}, respectively. FIGS. 21C and 21D illustrate that the male stops 412 (e.g., the first and second males stops 412a, 412b) can extend laterally away from the shuttle 14. FIGS. 21C and 21D illustrate that the male stops 412 (e.g., the first and second males stops 412a, 412b) can have a hammerhead shape such that a first end of the males stops 412 can be smaller (e.g., narrower) than a second end of the male stops 412. FIGS. 21C and 21D illustrate that the male stops 412 (e.g., the first and second male stops 412a, 412b) can be deflectable toward and away from a longitudinal axis of the shuttle 14. For example, FIGS. 21C and 21D illustrate that the male stops 412 (e.g., the first and second male stops 412a, 412b) can be deflectable toward and away from the opposite lateral side of the shuttle 14 than the lateral side of the shuttle 14 that the male stops 412 are on.

The shuttle 14 can have any combination of the male stops 412 shown in FIGS. 15A-21D. For example, the shuttle 14 can have 1, 2, 3, or 4 male stops 412. The shuttle 14 can have any combination of male stops 412. For example, the shuttle 14 can have any combination of radial male stops (e.g., the male stops 412 shown in FIGS. 16A-21B) and lateral male stops (e.g., the male stops 412 shown in FIGS. 21C and 21D), including, for example, two radial male stops (e.g., two of the male stops 412 shown in FIGS. 16A-21B), two lateral male stops (e.g., the male stops 412 shown in FIGS. 21C and 21D), one radial male stop on a first end of the shuttle 14 and one lateral male stop on a second end of the shuttle 14 (e.g., one of the male stops 412 shown in FIGS. 16A-21B and one of the male stops 412 shown in FIGS. 21C and 21D), and two radial male stops and two lateral male stops for a total of four male stops 412 (e.g., two of the male stops 412 shown in FIGS. 16A-21B and the two male stops 412 shown in FIGS. 21C and 21D). As another example, the shuttle 14 may not have any male stops 412.

FIGS. 16A-21D illustrate that the male stops 412 can be attached to, integrated with, or integrally formed with the shuttle 14 such that the one of the ends of the male stop 412 extends from the shuttle body 160, is a portion of the shuttle body 160, or both extends from the shuttle body 160 and is a portion of the shuttle body 160.

One or multiple portions of the male stops 412 can be engageable with a female stop 416. For example, the wider portion of the male stops 412 having a hammerhead shape (e.g., the hammerhead shape shown in FIGS. 21A-21D) can be engageable with a female stop 416 (e.g., can be moveable into and out of a female stop 416).

The male stop 412 can be bendable or deflectable, for example, about the base of the male stops 412 or anywhere along their length. For example, when the male stops 412 in FIGS. 21A-21D into and out of a female stop 416, the smaller portion (e.g., narrower portion) of the male stop 412 can deflect to allow the larger portion (e.g., wider portion) of the male stops 412 to pass into the female stop 416. In this way, the female stop 416 can passively retain the shuttle 14 in a jaw of the device 188 by passively retaining the male stop 412. The deflection of the base of the male stop 412 (e.g., the narrower portion of a hammerhead shape) can allow male stop 412 to pass into and out of a female stop 416, and can, for example, allow the tip of the male stop 412 (e.g., the wider portion of a hammerhead shape) to engage with and disengage with the female stop 416. In this way the male stops 412 can be a spring which can be deflectable (e.g., compressible) when the male stops 412 are moved out of a female stop 416.

Any male stop 412 can be replaced with a female stop 416 and vice versa. For example, any of the male and female stops 412, 416 disclosed herein can be swapped with each other such that the shuttle 14 has the female stops 416 and the jaws 801 have the male stops 412.

The first and second jaws 78, 80 can be upper and lower jaws, respectively. The first and second jaws 78, 80 can be lower and upper jaws, respectively. Any reference to an upper or lower jaw herein can be any of the jaws 801 disclosed, illustrated, and/or contemplated herein.

When the shuttle 14 is in the first jaw 78, the first male stop 412a (e.g., the first lateral male stop 412_{L1}) can be proximal the second male stop 412b (e.g., the second lateral male stop 412_{L2}) or vice versa. When the shuttle 14 is in the second jaw 80, the second male stop 412b (e.g., the second lateral male stop 412_{L2}) can be proximal the first male stop 412a (e.g., the first lateral male stop 412_{L1}).

When the first pusher 76 pushes the shuttle 14 from the track 64 to the track 66, the second male stop 412b (e.g., the second lateral male stop 412_{L2}) can enter the track 66 before the first male stop 412a (e.g., the first lateral male stop 412_{L1}) such that when the first pusher 76 pushes the shuttle 14 from the track 64 to the track 66, the second male stop 412b can be a leading male stop and the first male stop 412a can be a trailing male stop, or vice versa.

When the second pusher 86 pushes the shuttle 14 from the track 66 to the track 64, the first male stop 412a (e.g., the first lateral male stop 412_{L1}) can enter the track 64 before the second male stop 412b (e.g., the second lateral male stop 412_{L2}) such that when the second pusher 86 pushes the shuttle 14 from the track 66 to the track 64, the first male stop 412a can be a leading male stop and the second male stop 412b can be a trailing male stop, or vice versa.

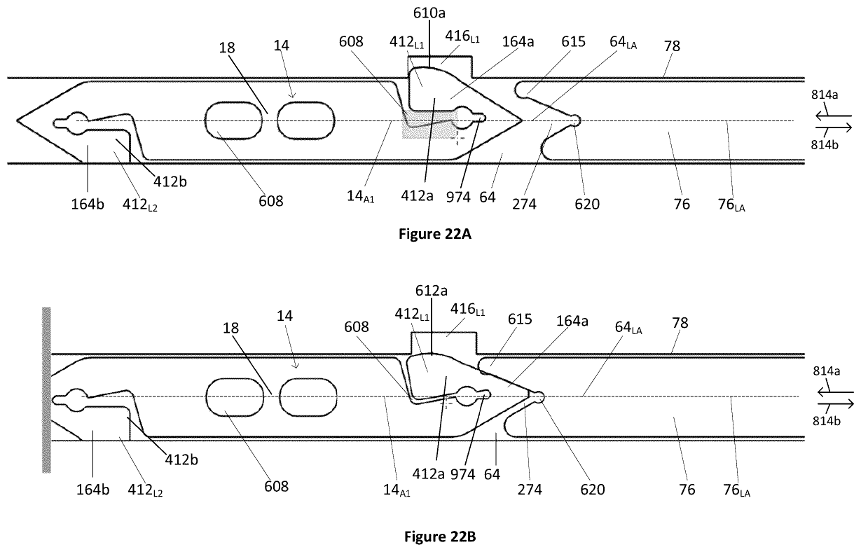

FIG. 22A illustrates the arrangement of features shown in FIG. 20D without the radial male stops extending from the suture holder 18 (e.g., radial male stops 412) and FIG. 22B illustrates that when the first pusher 76 is moved in direction 814a via the second trigger 804, the first pusher 76 can be engaged with the shuttle first tip 164a and move the shuttle first tip 164a from configuration 610a shown in FIG. 22A to configuration 612a shown in FIG. 22B. FIGS. 22A and 22B illustrate that the first pusher 76 can deflect the first lateral male stop 412_{L1} out of the first lateral female stop 416_{L1} toward the shuttle longitudinal axis 14_{A1} to release the shuttle 14 from the first jaw 78 and that further movement of the first pusher 76 in direction 814a can push the shuttle 14 into the opposite jaw.

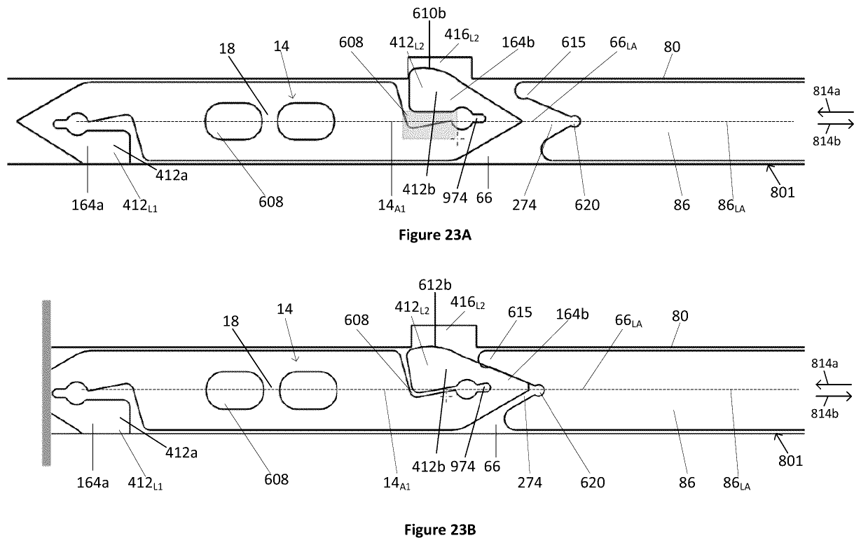

FIG. 23A illustrates the arrangement of features shown in FIG. 20E without the radial male stops extending from the suture holder 18 (e.g., radial male stops 412) and FIG. 23B illustrates that when the second pusher 86 is moved in direction 814a via the second trigger 804, the second pusher 86 can be engaged with the shuttle second tip 164b and move the shuttle second tip 164b from the configuration 610b shown in FIG. 23A to the configuration 612b shown in FIG. 23B. FIGS. 23A and 23B illustrate that the second pusher 86 can deflect the second lateral male stop 412_{L2} out of the second lateral female stop 416_{L2} toward the shuttle longitudinal axis 14_{A1} to release the shuttle 14 from the second jaw 80 and that further movement of the second pusher 86 in direction 814a can push the shuttle 14 into the opposite jaw.

FIGS. 22A-23B illustrate that the ends of the shuttle 14 can have a groove 974 (also shown but not labeled in FIGS. 20A-20E—for example, FIGS. 20A-20E illustrate that the ends of the shuttle 14 can each have two grooves 974). The groove 974 can manage the stress and strain of the male stops 412 (e.g., the first male stop 412a and the second male stop 412b) as the male stops 412 flex.

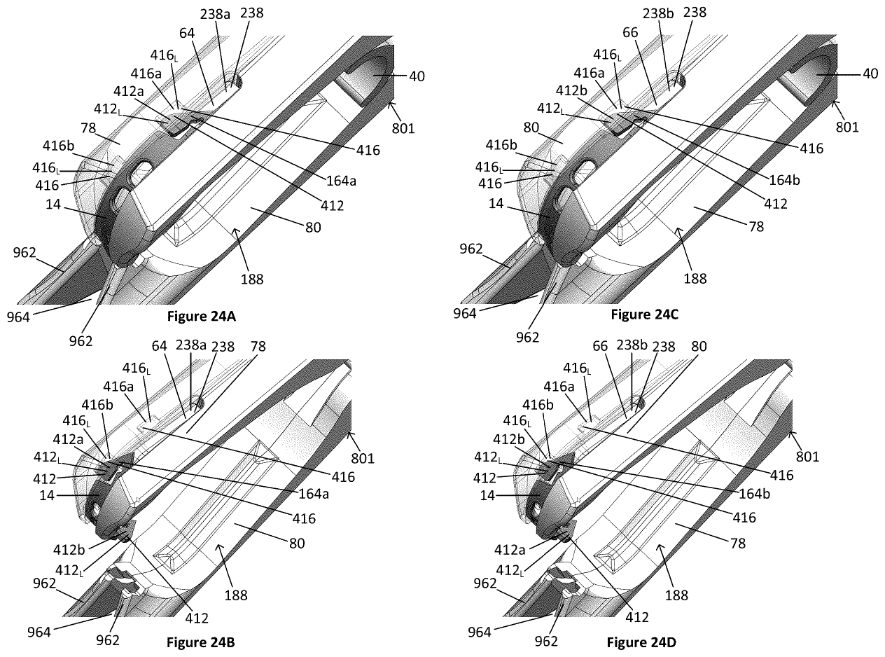

FIGS. 24A and 24B illustrate that the first jaw 78 can have multiple female stops 416, for example, a first female stop 416a and a second female stop 416b. The first female stop 416a can be proximal the second female stop 416b. The second female stop 416b can be closer to the tip of the first jaw 78 than the first female stop 416a. FIGS. 24A and 24B illustrate that the female stops 416 (e.g., the first and second female stops 416a, 416b) in the first jaw 78 can be recesses in the first jaw 78 that can extend away (e.g., laterally away) from the first jaw track 64. The female stops 416 in the first jaw 78 can be on the same side or on opposite sides (e.g., a first side and a second side) of the first jaw track 64. For example, example, FIGS. 24A and 24B illustrate that the first and second female stops 416a, 416b can be on the same side, for example, a first side (e.g., a first lateral side) of the first jaw track 64. As another example, the first female stop 416a can be on a first side (e.g., a first lateral side) and the second female stop 416b can be on a side (e.g., a second lateral side, a radial side) of the first jaw track 64. The female stops 416 in the first jaw 78 can be lateral female stops 416_{L}, can be radial female stops 416_{R}, can extend laterally and radially away from the first jaw track (e.g., can be a lateral and radial female stop), or any combination thereof. For example, FIGS. 24A and 24B illustrate that the first and second female stops 416a, 416b in the first jaw 78 can be lateral female stops 416_{L}.

FIGS. 24C and 24D illustrate that the second jaw 80 can have multiple female stops 416, for example, a first female stop 416a and a second female stop 416b. The first female stop 416a can be proximal the second female stop 416b. The second female stop 416b can be closer to the tip of the second jaw 80 than the first female stop 416a. FIGS. 24C and 24D illustrate that the female stops 416 (e.g., the first and second female stops 416a, 416b) in the second jaw 80 can be recesses in the second jaw 80 that can extend away (e.g., laterally away) from the second jaw track 66. The female stops 416 in the second jaw 80 can be on the same side or on opposite sides (e.g., a first side and a second side) of the second jaw track 66. For example, example, FIGS. 24C and 24D illustrate that the first and second female stops 416a, 416b can be on the same side, for example, a first side (e.g., a first lateral side) of the second jaw track 66. As another example, the first female stop 416a can be on a first side (e.g., a first lateral side) and the second female stop 416b can be on a side (e.g., a second lateral side, a radial side) of the second jaw track 66. The female stops 416 in the second jaw 80 can be lateral female stops 416_{L}, can be radial female stops 416_{R}, can extend laterally and radially away from the first jaw track (e.g., can be a lateral and radial female stop), or any combination thereof. For example, FIGS. 24C and 24D illustrate that the first and second female stops 416a, 416b in the second jaw 80 can be lateral female stops 416_{L}.

FIGS. 24A-24D illustrate that the female stops 416 in the first jaw 78 and the female stops 416 in the second jaw 80 can be on opposite sides of the device 188. For example, FIGS. 24A and 24B illustrate the female stops 416 in the first jaw 78 can be on a first lateral side of the device 188, and FIGS. 24C and 24D illustrate that the female stops 416 in the second jaw 80 can be on a second lateral side of the device 188. FIGS. 24A-24D illustrate that the first and second lateral sides of the device 188 can be opposite each other. FIGS. 24A-24D illustrate that the female stops 416 in the first jaw 78 and the female stops 416 in the second jaw 80 can extend in opposite directions. For example, FIGS. 24A and 24B illustrate that the female stops 416 in the first jaw 78 can be on a first side of the first jaw track 64 and FIGS. 24C and 24D illustrate that the female stops 416 in the second jaw 80 can be on a second side of the second jaw track 66. The female stops 416 in the first jaw 78 can extend in a first direction away from the first jaw track 64 (e.g., in a first direction away from the device longitudinal axis, in a first direction away from a longitudinal axis of the first jaw track 64) and the female stops 416 in the second jaw 80 can extend in a second direction away from the second jaw track 66 (e.g., in a second direction away from the device longitudinal axis, in a second direction away from a longitudinal axis of the second jaw track 66). The first direction can be opposite the second direction.

FIGS. 24A-24D illustrate that the shuttle 14 can have multiple male stops 412, for example, a first male stop 412a and a second male stop 412b. The first male stop 412a and the second male stop 412b can be on opposite ends of the shuttle 14. The male stops 412 of the shuttle 14 can be lateral male stops 412_{L}, radial male stops 412_{R}, can be a combination of both (e.g., such that one or more of the male stops 412 extends laterally and radially away from the shuttle longitudinal axis), or any combination thereof. For example, FIGS. 24A-24D illustrate that the first and second male stops 412a, 412b of the shuttle 14 can be lateral male stops 412_{L}.

The same male stop 412 can be engageable with the first and second female stops 416a, 416b in the first jaw 78. For example, FIGS. 24A and 24B illustrate that the same male stop 412 (e.g., the first male stop 412a) can be engageable with the first and second female stops 416a, 416b in the first jaw 78, and FIGS. 24C and 24D illustrate that the same male stop 412 (e.g., the second male stop 412b) can be engageable with the first and second female stops 416a, 416b in the second jaw 80. As another example, different male stops 412 can be engageable with the first and second female stops 416a, 416b in the first and second jaws 78, 80. For example, a first male stop 412a can be engageable with the first female stop 416a in the first jaw 78, a second male stop 412b can be engageable with the second female stop 416b in the first jaw 78, the second male stop 412b can be engageable with a first female stop 416a in the second jaw 80, and the first male stop 412a can be engageable with a second female stop 416b in the second jaw 80.

FIGS. 24A and 24B illustrate that the first male stop 412a can be movable into and out of the first female stop 416a in the first jaw 78. FIGS. 24A and 24B illustrate that the first male stop 412a can be movable into and out of the second female stop 416b in the first jaw 78. When the first male stop 412a is in the first female stop 416a in the first jaw 78, the shuttle 14 can be passively retained in the first jaw 78. When the first male stop 412a is in the second female stop 416b in the first jaw 78, the shuttle 14 can be passively retained in the first jaw 78. FIGS. 24A and 24B illustrate that the first male stop 412a can be releasably engageable with the first female stop 416a in the first jaw 78. FIGS. 24A and 24B illustrate that the first male stop 412a can be releasably engageable with the second female stop 416b in the first jaw 78.

FIGS. 24C and 24D illustrate that the second male stop 412b can be movable into and out of the first female stop 416a in the second jaw 80. FIGS. 24C and 24D illustrate that the second male stop 412b can be movable into and out of the second female stop 416b in the second jaw 80. When the second male stop 412b is in the first female stop 416a in the second jaw 80, the shuttle 14 can be passively retained in the second jaw 80. When the second male stop 412b is in the second female stop 416b in the second jaw 80, the shuttle 14 can be passively retained in the second jaw 80. FIGS. 24C and 24D illustrate that the second male stop 412b can be releasably engageable with the first female stop 416a in the second jaw 80. FIGS. 24C and 24D illustrate that the second male stop 412b can be releasably engageable with the second female stop 416b in the second jaw 80.

FIGS. 24A and 24C illustrate the jaws 801 in a closed configuration in which the shuttle 14 can be passed between the jaws 801 by the pushers 803.

FIGS. 24B and 24D illustrate the jaws 801 in an open configuration in which the shuttle has been caught (e.g., passively caught) and prevented from falling out of the jaws 801.

FIGS. 24A and 24B illustrate that the male stop 412 on a first end of the shuttle 14 (e.g., the first male stop 412a) can deflect into and out of the female shuttle stops 416 (e.g., the first female stop 416a and the second female stop 416b) in the first jaw 78 as the shuttle 14 is pushed into and out of the first jaw 78. As the shuttle 14 is pushed into the first jaw 78 by the second pusher 86, FIG. 24A illustrates that the second pusher 86 can advance the first male stop 412a into and out of the distal female stop in the first jaw 78 (e.g., the second female stop 416b in the first jaw 78) and into the proximal female stop in the first jaw 78 (e.g., the first female stop 416a in the first jaw 78). The first male stop 412a can move into and out of the female stops 416, for example, by deflecting (e.g., bending, rotating, or both) about its base, and/or as described elsewhere herein.

As the first male stop 412a is advanced (e.g., pushed) past the second female stop 416b in the first jaw 78 toward the first female stop 416a in the first jaw 78, the first male stop 412a can move into and out of the second female stop 416b such that the engagement between the first male stop 412a and the second female stop 416b in the first jaw 78 can allow the second pusher 86 to advance the shuttle 14 into the first jaw 78 until the first male stop 412a is caught (e.g., becomes engaged with) the first female stop 416a. The second female stop 416b in the first jaw 78 thereby may not prevent the shuttle 14 from being fully advanced into the first jaw 78.

As the first male stop 412a moves into and out of the second female stop 416b in the first jaw 78 as the first male stop 412a is advanced toward the first female stop 416a, the first male stop 412a can move away from the first jaw track 64 (e.g., away from the longitudinal axis of the first jaw track 64) as the first male stop 412a moves into the second female stop 416b and can move toward the first jaw track 64 (e.g., toward the longitudinal axis of the first jaw track 64) as the first male stop 412a moves out of the second female stop 416b. When the first male stop 412a is in (e.g., is fully in) the second female stop 416b in the first jaw 78, the first male stop 412a can be in a non-deflected configuration or in a partially deflected configuration (e.g., a partially compressed configuration). For example, when the first male stop 412a is in the second female stop 416b in the first jaw 78, the first male stop 412a can have the male stop first configuration.

As the first male stop 412a is advanced by the second pusher 86 from the second female stop 416b to the first female stop 416a in the first jaw 78, the first male stop 412a can be between the first and second female stops 416a, 416b. When the first male stop 412a is between the first and second female stops 416a, 416b in the first jaw 78, the first male stop 412a can be in a partially deflected configuration or in a fully deflected configuration (e.g., a partially or fully compressed configuration). For example, when the first male stop 412a is between the first and second female stops 416a, 416b in the first jaw 78, the first male stop 412a can have the male stop second configuration. The first male stop 412a can be closer to the first jaw track 64 (e.g., closer to the longitudinal axis of the first jaw track 64) when the first male stop 412a is between the first and second female stops 416a, 416b than when the first male stop 412a is in the second female stop 416b, than when the first male stop 412a is in the first female stop 416a, or both when the first male stop 412a is in the second female stop 416b and when the first male stop 412a is in the first female stop 416a.

As the first male stop 412a is advanced (e.g., pushed) into the first female stop 416a in the first jaw 78, the first male stop 412a can move into the first female stop 416a such that the first female stop 416a can catch (e.g., become engaged with) the first male stop 412a. The first female stop 416a can, for example, passively catch the first male stop 412a. As the first male stop 412a moves into the first female stop 416a in the first jaw 78 as the shuttle 14 is advanced by the second pusher 86, the first male stop 412a can move away from the first jaw track 64 (e.g., away from the longitudinal axis of the first jaw track 64) as the first male stop 412a moves into the first female stop 416a. When the first male stop 412a is in (e.g., is fully in) the first female stop 416a in the first jaw 78, the first male stop 412a can be in a non-deflected configuration or in a partially deflected configuration (e.g., the same or different configuration as the configuration the first male stop 412a has when in the second female stop 416b). For example, when the first male stop 412a is in the first female stop 416a in the first jaw 78, the first male stop 412a can have the male stop first configuration.

When the first male stop 412a is in the first female stop 416a, the first female stop 416a in the first jaw 78 can retain (e.g., passively retain) the first male stop 412a until the first pusher 76 is advanced to move (e.g., push) the shuttle 14 out of the first jaw 78 and into the second jaw 80.

As the shuttle 14 is pushed out of the first jaw 78 by the first pusher 76, FIG. 24A illustrates that the first pusher 76 can advance the first male stop 412a out of the first female stop 416a in the first jaw 78 and can advance the first male stop 412a past the second female stop 416b in the first jaw 78 to move the shuttle 14 into the second jaw 80.

As the first male stop 412a is advanced (e.g., pushed) out of the first female stop 416a in the first jaw 78 by the first pusher 76, the first male stop 412a can move toward the first jaw track 64 (e.g., toward the longitudinal axis of the first jaw track 64) as the first male stop 412a is forced out the first female stop 416a by the first pusher 76 and into the first jaw track 64 between the first and second female stops 416a, 416b in the first jaw 78. The first pusher 76 can continue to push the shuttle 14 past the second female stop 416b and into the second jaw 80.

As the first male stop 412a is moved past the second female stop 416b in the first jaw 78 as the first male stop 412a is advanced toward the second jaw 80 by the first pusher 76, the first male stop 412a can move into and out of the second female stop 416b. For example, the first male stop 412a can move away from the first jaw track 64 (e.g., away from the longitudinal axis of the first jaw track 64) as the first male stop 412a moves into the second female stop 416b and can move toward the first jaw track 64 (e.g., toward the longitudinal axis of the first jaw track 64) as the first male stop 412a moves out of the second female stop 416b. When the first male stop 412a is in (e.g., is fully in) the second female stop 416b, the first male stop 412a can be in a non-deflected configuration or in a partially deflected configuration (e.g., a partially compressed configuration).

FIGS. 24A and 24B illustrate that first female stop 416a in the first jaw 78 can be a primary catch and that the second female stop 416b in the first jaw 78 can be a secondary catch. For example, FIG. 24B illustrates that the second female stop 416b in the first jaw 78 can catch (e.g., become engaged with) the shuttle 14 if the shuttle 14 is not advanced far enough into the first jaw 78, if the first male stop 412a slips out of the first female stop 416a, or both. For example, the second female stop 416b can catch (e.g., passively catch) the first male stop 412a as the shuttle 14 moves out (e.g., slips out) of the first jaw 78 without the assistance of the first pusher 76. As another example, the second female stop 416b can catch (e.g., passively catch) the first male stop 412a as the shuttle 14 falls out of the first jaw 78 if the jaws 801 are opened when the shuttle 14 has not been advanced far enough into the first jaw 78 by the second pusher 86 to have been caught by the first female stop 416a in the first jaw 78 (e.g., if the jaws 801 are opened when the first stop 412a is between the first and second female stops 416a, 416b).

FIGS. 24C and 24D illustrate that the male stop 412 on a second end of the shuttle 14 (e.g., the second male stop 412b) can deflect into and out of the female shuttle stops 416 (e.g., the first female stop 416a and the second female stop 416b) in the second jaw 80 as the shuttle 14 is pushed into and out of the second jaw 80. As the shuttle 14 is pushed into the second jaw 80 by the first pusher 76, FIG. 24C illustrates that the first pusher 76 can advance the second male stop 412b into and out of the distal female stop in the second jaw 80 (e.g., the second female stop 416b in the second jaw 80) and into the proximal female stop in the second jaw 80 (e.g., the first female stop 416a in the second jaw 80). The second male stop 412b can move into and out of the female stops 416, for example, by deflecting (e.g., bending, rotating, or both) about its base, and/or as described elsewhere herein.

As the second male stop 412b is advanced (e.g., pushed) past the second female stop 416b in the second jaw 80 toward the first female stop 416a in the second jaw 80, the second male stop 412b can move into and out of the second female stop 416b such that the engagement between the second male stop 412b and the second female stop 416b in the second jaw 80 can allow the first pusher 76 to advance the shuttle 14 into the second jaw 80 until the second male stop 412b is caught (e.g., becomes engaged with) the first female stop 416a. The second female stop 416b in the second jaw 80 thereby may not prevent the shuttle 14 from being fully advanced into the second jaw 80.

As the second male stop 412b moves into and out of the second female stop 416b in the second jaw 80 as the second male stop 412b is advanced toward the first female stop 416a, the second male stop 412b can move away from the second jaw track 66 (e.g., away from the longitudinal axis of the second jaw track 66) as the second male stop 412b moves into the second female stop 416b and can move toward the second jaw track 66 (e.g., toward the longitudinal axis of the second jaw track 66) as the second male stop 412b moves out of the second female stop 416b. When the second male stop 412b is in (e.g., is fully in) the second female stop 416b in the second jaw 80, the second male stop 412b can be in a non-deflected configuration or in a partially deflected configuration (e.g., a partially compressed configuration). For example, when the second male stop 412b is in the second female stop 416b in the second jaw 80, the second male stop 412b can have the male stop first configuration.

As the second male stop 412b is advanced by the first pusher 76 from the second female stop 416b to the first female stop 416a in the second jaw 80, the second male stop 412b can be between the first and second female stops 416a, 416b. When the second male stop 412b is between the first and second female stops 416a, 416b in the second jaw 80, the second male stop 412b can be in a partially deflected configuration or in a fully deflected configuration (e.g., a partially or fully compressed configuration). For example, when the second male stop 412b is between the first and second female stops 416a, 416b in the second jaw 80, the second male stop 412b can have the male stop second configuration. The second male stop 412b can be closer to the second jaw track 66 (e.g., closer to the longitudinal axis of the second jaw track 66) when the second male stop 412b is between the first and second female stops 416a, 416b than when the second male stop 412b is in the second female stop 416b, than when the second male stop 412b is in the first female stop 416a, or both when the second male stop 412b is in the second female stop 416b and when the second male stop 412b is in the first female stop 416a.

As the second male stop 412b is advanced (e.g., pushed) into the first female stop 416a in the second jaw 80, the second male stop 412b can move into the first female stop 416a such that the first female stop 416a can catch (e.g., become engaged with) the second male stop 412b. The first female stop 416a can, for example, passively catch the second male stop 412b. As the second male stop 412b moves into the first female stop 416a in the second jaw 80 as the shuttle 14 is advanced by the first pusher 76, the second male stop 412b can move away from the second jaw track 66 (e.g., away from the longitudinal axis of the second jaw track 66) as the second male stop 412b moves into the first female stop 416a. When the second male stop 412b is in (e.g., is fully in) the first female stop 416a in the second jaw 80, the second male stop 412b can be in a non-deflected configuration or in a partially deflected configuration (e.g., the same or different configuration as the configuration the second male stop 412b has when in the second female stop 416b). For example, when the second male stop 412b is in the first female stop 416a in the second jaw 80, the second male stop 412b can have the male stop first configuration.

When the second male stop 412b is in the first female stop 416a, the first female stop 416a in the second jaw 80 can retain (e.g., passively retain) the second male stop 412b until the second pusher 86 is advanced to move (e.g., push) the shuttle 14 out of the second jaw 80 and into the first jaw 78.

As the shuttle 14 is pushed out of the second jaw 80 by the second pusher 86, FIG. 24C illustrates that the second pusher 86 can advance the second male stop 412b out of the first female stop 416a in the second jaw 80 and can advance the second male stop 412b past the second female stop 416b in the second jaw 80 to move the shuttle 14 into the first jaw 78.

As the second male stop 412b is advanced (e.g., pushed) out of the first female stop 416a in the second jaw 80 by the second pusher 86, the second male stop 412b can move toward the second jaw track 66 (e.g., toward the longitudinal axis of the second jaw track 66) as the second male stop 412b is forced out the first female stop 416a by the second pusher 86 and into the second jaw track 66 between the first and second female stops 416a, 416b in the second jaw 80. The second pusher 86 can continue to push the shuttle 14 past the second female stop 416b and into the first jaw 78.

As the second male stop 412b is moved past the second female stop 416b in the second jaw 80 as the second male stop 412b is advanced toward the first jaw 78 by the second pusher 86, the second male stop 412b can move into and out of the second female stop 416b. For example, the second male stop 412b can move away from the second jaw track 66 (e.g., away from the longitudinal axis of the second jaw track 66) as the second male stop 412b moves into the second female stop 416b and can move toward the second jaw track 66 (e.g., toward the longitudinal axis of the second jaw track 66) as the second male stop 412b moves out of the second female stop 416b. When the second male stop 412b is in (e.g., is fully in) the second female stop 416b, the second male stop 412b can be in a non-deflected configuration or in a partially deflected configuration (e.g., a partially compressed configuration).

FIGS. 24C and 24D illustrate that first female stop 416a in the second jaw 80 can be a primary catch and that the second female stop 416b in the second jaw 80 can be a secondary catch. For example, FIG. 24D illustrates that the second female stop 416b in the second jaw 80 can catch (e.g., become engaged with) the shuttle 14 if the shuttle 14 is not advanced far enough into the second jaw 80, if the second male stop 412b slips out of the first female stop 416a, or both. For example, the second female stop 416b can catch (e.g., passively catch) the second male stop 412b as the shuttle 14 moves out (e.g., slips out) of the second jaw 80 without the assistance of the second pusher 86. As another example, the second female stop 416b can catch (e.g., passively catch) the second male stop 412b as the shuttle 14 falls out of the second jaw 80 if the jaws 801 are opened when the shuttle 14 has not been advanced far enough into the second jaw 80 by the first pusher 76 to have been caught by the first female stop 416a in the second jaw 80 (e.g., if the jaws 801 are opened when the first stop 412a is between the first and second female stops 416a, 416b).

FIGS. 24A-24D illustrate that the jaws 801 can have four female stops 416 such that each jaw can have a first female stop 416a and a second female stop 416b. The first female stop 416a can be proximal the second female stop 416b. In the first jaw 78, for example, the first female stop 416a can be proximal the second female stop 416b, for example, along the first jaw track 64. In the second jaw 80, for example, the first female stop 416a can be proximal the second female stop 416b, for example, along the second jaw track 66. The second female stops 416b can be fail safe stops, or backup stops, that can inhibit or prevent the shuttle 14 from falling out of the first jaw 78 or the second jaw 80 if the shuttle 14 does not engage with the first female stop 416a when the shuttle 14 is pushed into the first jaw 78 or the second jaw 80, respectively. The first female stops 416a can be primary catches for the shuttle 14 and the second female stops 416b can be secondary catches for the shuttle 14. FIGS. 24A-24D illustrate that the first and second female stops 416a, 416b can be lateral female stops 416_{L}. As another example, the first and second female stops 416a, 416b can be radial female stops 416_{R}. FIGS. 24A-24D illustrate that the first and second male stops 412a, 412b can be lateral male stops 412_{L}. As another example, the first and second male stops 412a, 412b can be radial male stops 412_{R}.

FIGS. 24A and 24B illustrate that if the shuttle 14 is not pushed far enough into the first jaw 78 or if the shuttle 14 slips out of the first female stop 416a in the first jaw 78, the secondary catch (e.g., the second female stop 416b) can catch the proximal male stop 412 (e.g., the first male stop 412a or the second male stop 412b) to prevent the shuttle 14 from completely falling out of the first jaw 78 and becoming detached from the device 188. As another example, FIGS. 24A and 24B illustrate that if the shuttle first tip 164a is not pushed far enough into the first jaw 78 or if the male stop 412 of the shuttle first tip 164a (e.g., the first male stop 412a) slips out of the first female stop 416a, the secondary catch (e.g., the second female stop 416b) can catch one of the male stops 412 (e.g., the male stop 412 of the shuttle first tip 164a) to prevent the shuttle 14 from completely falling out of the first jaw 78 and becoming detached from the device 188.

FIGS. 24A and 24B illustrate that when the shuttle 14 is in the first jaw 78, the proximal male stop 412 can be the first male stop 412a. FIG. 24A illustrates that the first male stop 412a can be pushed past the second female stop 416b until the first male stop 412a is engaged with the first female stop 416a. FIG. 24A illustrates an exemplary arrangement before the first male stop 412a slips out of the first female stop 416a and is caught by the second female stop 416b as shown in FIG. 24B. The second female stop 416b in the first jaw 78 can catch the shuttle 14 (e.g., the first male stop 412a) when the jaws 801 are in an open configuration or in a closed configuration. For example, FIG. 24B illustrates the shuttle 14 in a caught configuration in which the jaws 801 are in an open configuration.

FIGS. 24C and 24D illustrate that if the shuttle 14 is not pushed far enough into the second jaw 80 or if the shuttle 14 slips out of the first female stop 416a in the second jaw 80, the secondary catch (e.g., the second female stop 416b) can catch the proximal male stop 412 (e.g., the first male stop 412a or the second male stop 412b) to prevent the shuttle 14 completely falling out of the second jaw 80 and becoming detached from the device 188. As another example, FIGS. 24C and 24D illustrate that if the shuttle second tip 164b is not pushed far enough into the second jaw 80 or if the male stop 412 of the shuttle second tip 164b (e.g., the second male stop 412b) slips out of the first female stop 416a, the secondary catch (e.g., the second female stop 416b) can catch one of the male stops 412 (e.g., the male stop 412 of the shuttle second tip 164b) to prevent the shuttle 14 from completely falling out of the second jaw 80 and becoming detached from the device 188. FIGS. 24C and 24D illustrate that when the shuttle 14 is in the second jaw 80, the proximal male stop 412 can be the second male stop 412b. FIG. 24C illustrates that the second male stop 412b can be pushed past the second female stop 416b until the second male stop 412b is engaged with the first female stop 416a. FIG. 24C illustrates an exemplary arrangement before the second male stop 412b slips out of the first female stop 416a and is caught by the second female stop 416b as shown in FIG. 24D. The second female stop 416b in the second jaw 80 can catch the shuttle 14 (e.g., the second male stop 412b) when the jaws 801 are in an open configuration or in a closed configuration. For example, FIG. 24D illustrates the shuttle 14 in a caught configuration in which the jaws 801 are in an open configuration.

FIGS. 24A-24D thereby illustrate that second female stop 416b in the first jaw 78 and the second female stop 416b in the second jaw 80 can inhibit or prevent the shuttle 14 from becoming detached from the jaws 801.

FIG. 24A illustrates that when the first male stop 412a is engaged with the first female stop 416a in the first jaw 78, the second male stop 412b can have a deflected configuration. The moment of the deflected second male stop 412b can torque the shuttle 14 in the first jaw track 64, which can decrease the hold between the first male stop 412a and the first female stop 416a that is shown in FIG. 24A. FIG. 24B illustrates that when the shuttle 14 is caught by the second female stop 416b in the first jaw 78, the second male stop 412b can be outside of the jaws 801, which can remove the moment created by the second male stop 412b in the first jaw 78. This can result in a tighter hold between the first male stop 412a and the second female stop 416b than between the first male stop 412a and the first female stop 416a. When the shuttle 14 is in the caught configuration as shown in FIG. 24B, the jaws 801 can be closed before removing the device 188 from the patient. As the jaws 801 are closed, the shuttle 14 can bend and deflect out of the way, for example, into the space between the jaws 801. Once the device 188 is removed from the patient, the shuttle 14 can be removed from the device 188 and reloaded or a new shuttle 14 can be loaded into the first jaw 78 or into the second jaw 80. As another example, when the first male stop 412a is engaged with the first female stop 416a in the first jaw 78, the second male stop 412b can have a non-deflected configuration inside the first jaw 78 (e.g., in the first jaw track 64) or outside the first jaw 78. In such variations, the hold between the first male stop 412a and the second female stop 416b and between the first male stop 412a and the first female stop 416a can be the same.

FIG. 24C illustrates that when the second male stop 412b is engaged with the first female stop 416a in the second jaw 80, the first male stop 412a can have a deflected configuration. The moment of the deflected first male stop 412a can torque the shuttle 14 in the second jaw track 66, which can decrease the hold between the second male stop 412b and the first female stop 416a that is shown in FIG. 24C. FIG. 24D illustrates that when the shuttle 14 is caught by the second female stop 416b in the second jaw 80, the first male stop 412a can be outside of the jaws 801, which can remove the moment created by the first male stop 412a in the second jaw 80. This can result in a tighter hold between the second male stop 412b and the second female stop 416b than between the second male stop 412b and the first female stop 416a. When the shuttle 14 is in the caught configuration as shown in FIG. 24D, the jaws 801 can be closed before removing the device 188 from the patient. As the jaws 801 are closed, the shuttle 14 can bend and deflect out of the way, for example, into the space between the jaws 801. Once the device 188 is removed from the patient, the shuttle 14 can be removed from the device 188 and reloaded or a new shuttle 14 can be loaded into the first jaw 78 or into the second jaw 80. As another example, when the second male stop 412b is engaged with the first female stop 416a in the second jaw 80, the first male stop 412a can have a non-deflected configuration inside the second jaw 80 (e.g., in the second jaw track 66) or outside the second jaw 80. In such variations, the hold between the second male stop 412b and the second female stop 416b and between the second male stop 412b and the first female stop 416a can be the same.

The pushers 803 (e.g., the first pusher 76 and the second pusher 86) in FIGS. 24A-24D are shown transparent for illustrative purposes only.

Half of the first jaw 78 is shown transparent in FIGS. 24A and 24B so that the female stops 416 can be seen.

Half of the second jaw 80 is shown transparent in FIGS. 24C and 24D so that the female stops 416 can be seen.

FIGS. 24A-24D illustrate that the first jaw 78, the second jaw 80, or the first and second jaws 78, 80 can have tissue guides 962.

FIGS. 24A-24D illustrate that the shuttle 14 can be passively retainable in the jaws 801 via the male and female stops 412, 416. As another example, the shuttle 14 can be actively retainable in the jaws 801 via the male and female stops 412, 416, for example, using one or more controls on the handle of the device 188 that are linked to one or more of the male stops 412 and/or to one or more of the female stops 416, for example, to actively engage and/or actively disengage the male and female stops 412, 416 from each other as the shuttle 14 is passed between the jaws 801.

FIG. 24A illustrates that when the first male stop 412a is engaged with the first female stop 416a in first jaw 78, the second female stop 416b in the first jaw 78 can be between (e.g., halfway between) the first male stop 412a and the second male stop 412b. As another example, when the first male stop 412a is engaged with the first female stop 416a in first jaw 78, the second female stop 416b in the first jaw 78 can be closer to the second male stop 412b than to the first male stop 412a. As yet another example, when the first male stop 412a is engaged with the first female stop 416a in first jaw 78, the second female stop 416b in the first jaw 78 can be closer to the first male stop 412a than to the second male stop 412b.

FIG. 24C illustrates that when the second male stop 412b is engaged with the first female stop 416a in second jaw 80, the second female stop 416b in the second jaw 80 can be between (e.g., halfway between) the second male stop 412b and the first male stop 412a.

As another example, when the second male stop 412b is engaged with the first female stop 416a in second jaw 80, the second female stop 416b in the second jaw 80 can be closer to the first male stop 412a than to the second male stop 412b. As yet another example, when the second male stop 412b is engaged with the first female stop 416a in second jaw 80, the second female stop 416b in the second jaw 80 can be closer to the second male stop 412b than to the first male stop 412a.

FIGS. 20B, 20C, 25A, and 25B illustrate that the pushers 803 can keep the trailing male stop 412 closed (e.g., configurations 612a and 612b in FIGS. 20B and 20C, respectively) as the pushers 803 move the shuttle 14 between the jaws 801. When the shuttle 14 is moved by the first pusher 76 into the second jaw 80, the trailing male stop 412 can be, for example, the first male stop 412a (e.g., the first male stop 412a in FIGS. 24A-24D). When the shuttle 14 is moved by the second pusher 86 into the first jaw 78, the trailing male stop 412 can be, for example, the second male stop 412b (e.g., the second male stop 412b in FIGS. 24A-24D).

Keeping the trailing male stop 412 closed as the shuttle 14 is moved between the jaws 801 can inhibit or prevent the trailing male stop 412 from grabbing onto tissue (e.g., the tissue 74) as the shuttle 14 is pushed into the opposite jaw (e.g., as shown in FIG. 25A, can inhibit or prevent the trailing male stop 412 from hitting or grabbing onto the opposite jaw (e.g., as shown in FIG. 25B) in such a way that would prevent the trailing male stop 412 from entering the opposite jaw, or can inhibit or prevent both. As another example, the trailing male stop 412 can enter the opposite jaw and may not grab onto tissue without keeping the trailing male stop 412 closed as the shuttle 14 is moved between the jaws 801.

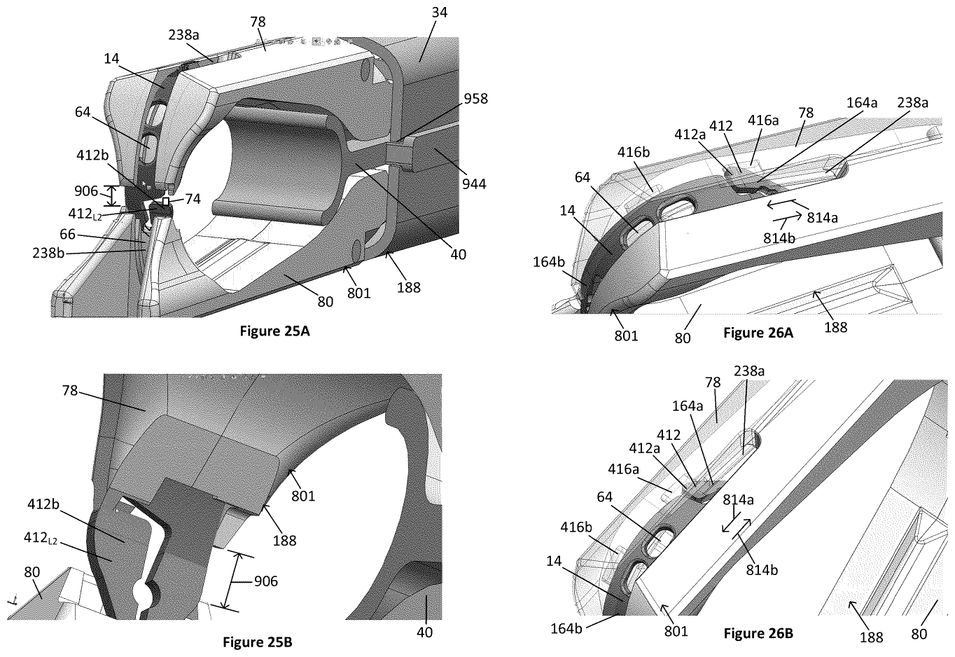

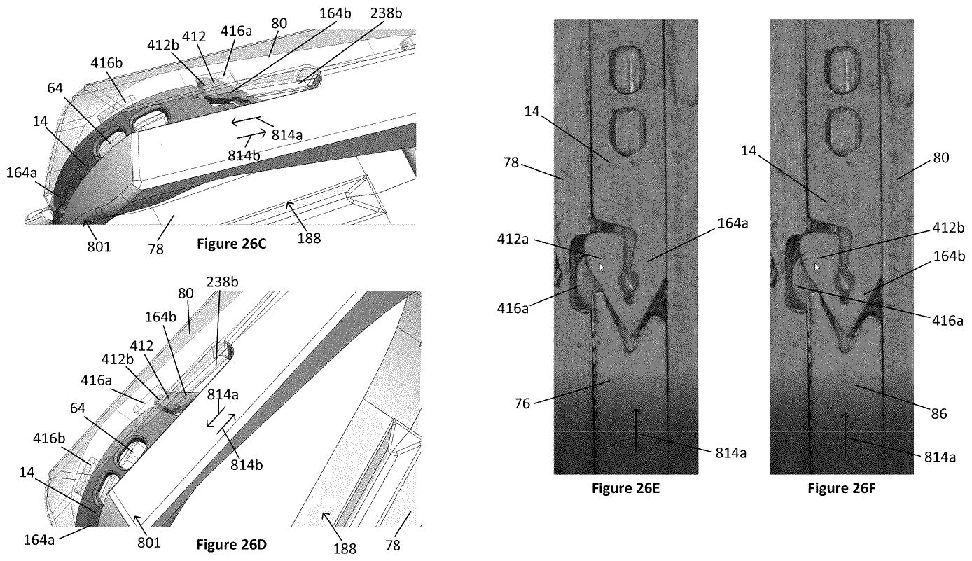

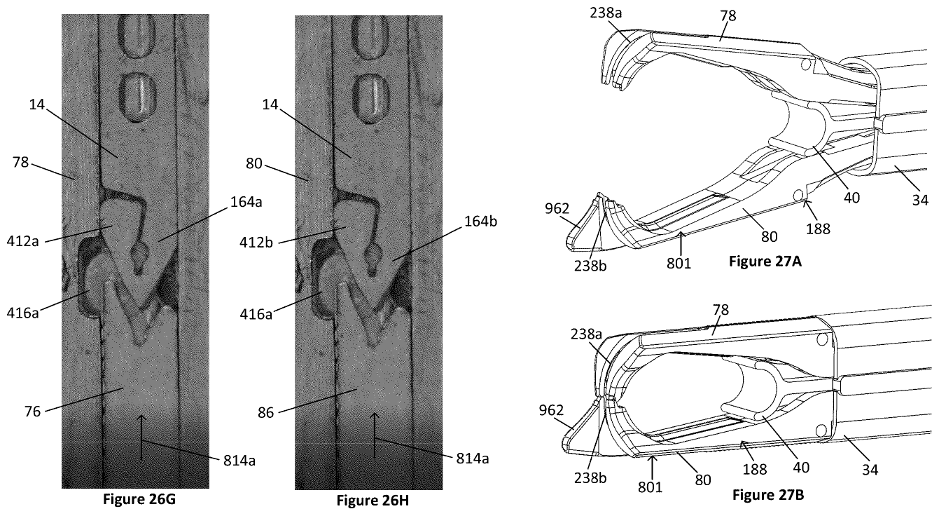

FIGS. 26A-26D illustrate that when the jaws 801 are moved from an open configuration to closed configuration against tissue, the proximal male stop 412 (e.g., the first male stop 412a or the second male stop 412b) can be proximally pushed (e.g., bumped backwards in direction 814b) away from the front edge (e.g., distal edge) of the first female stop 416a that the proximal male stop 412 is engaged with. Pushing (e.g., bumping) the proximal male stop 412 proximally as the jaws 801 are closed can inhibit or prevent the proximal male stop 412 from getting locked in the first female stop 416a.

The proximal male stop 412 can be in a locked configuration in the first female stop 416a in the first jaw 78, for example, if the first pusher 76 cannot close the proximal male stop 412 as the first pusher 76 is moved in direction 814a or as a force is applied against the proximal male stop 412 in direction 814a by the first pusher 76. A locked configuration can occur in the first jaw 78, for example, if the proximal male stop 412 is in contact with the front edge (e.g., distal edge) of the first female stop 416a in the first jaw 78 when the first pusher 76 is pushed against proximal male stop 412. The proximal male stop 412 can be moved away from or unlocked from a locked configuration in the first female stop 416a in the first jaw 78 by clamping the jaws 801 against tissue, by advancing the second pusher 86 into the first jaw 78, or both.

The proximal male stop 412 can be in a locked configuration in the first female stop 416a in the second jaw 80, for example, if the second pusher 86 cannot close the proximal male stop 412 as the second pusher 86 is moved in direction 814a or as a force is applied against the proximal male stop 412 in direction 814a by the second pusher 86. A locked configuration can occur in the second jaw 80, for example, if the proximal male stop 412 is in contact with the front edge (e.g., distal edge) of the first female stop 416a in the second jaw 80 when the second pusher 86 is pushed against proximal male stop 412. The proximal male stop 412 can be moved away from or unlocked from a locked configuration in the first female stop 416a in the second jaw 80 by clamping the jaws 801 against tissue, by advancing the first pusher 76 into the second jaw 80, or both.

FIGS. 26A and 26B illustrate, for example, that as the jaws 801 are clamped against tissue (e.g., the tissue 74), the tissue can push the shuttle 14 proximally in the first jaw track 64 in direction 814b to push (e.g., bump) the first male stop 412a proximally away from the front end (e.g., distal end) of the first female stop 416a. For example, as the jaws 801 are closed, tissue can push the shuttle exposed portion 423 of the shuttle first tip 164a into the first jaw 78 to bump the first male stop 412a toward a proximal end of the first female stop 416a. As the jaws 801 are closed, the tissue can proximally move the shuttle 14 by a bump distance in the first jaw 78 (also referred to as a pushback distance in the first jaw 78). For example, as the jaws 801 are closed, the tissue can move the proximal male stop 412 closer to the rear end (e.g., proximal end) of the first female stop 416a in the first jaw 78 by the bump distance. As another example, once the jaws 801 are closed, the first second pusher 86 can be advanced into the first jaw 78 to proximally move the shuttle 14 in direction 814b by the bump distance in the first jaw 78. The bump distance can be, for example, 1 mm to 6 mm, or more narrowly, from 1 mm to 4 mm, or more narrowly, from 1 mm to 3 mm, including every 1 mm increment within these ranges (e.g., 1 mm, 2 mm, 3 mm, 4 mm, 5 mm, 6 mm). FIG. 26A illustrates the proximal male stop 412 (e.g., the first male stop 412a) in the first female stop 416a before being bumped back by the bump distance, and FIG. 26B illustrates the proximal male stop 412 (e.g., the first male stop 412a) in the first female stop 416a after being bumped back by the bump distance. When the first male stop 412a is in the position shown in FIG. 26B, the first pusher 76 can close the first male stop 412a (e.g., from the male stop first configuration to the male stop second configuration) and push the shuttle 14 to the second jaw 80.

For example, FIGS. 26C and 26D illustrate that as the jaws 801 are clamped against tissue (e.g., the tissue 74), the tissue can push the shuttle 14 proximally in the second jaw track 66 in direction 814b to push (e.g., bump) the second male stop 412b proximally away from the front end (e.g., distal end) of the first female stop 416a. For example, as the jaws 801 are closed, tissue can push the shuttle exposed portion 423 of the shuttle second tip 164b into the second jaw 80 to bump the second male stop 412b toward a proximal end of the first female stop 416a. As the jaws 801 are closed, the tissue can proximally move the shuttle 14 by a bump distance in the second jaw 80 (also referred to as a pushback distance in the second jaw 80). For example, as the jaws 801 are closed, the tissue can move the proximal male stop 412 closer to the rear end (e.g., proximal end) of the first female stop 416a in the second jaw 80 by the bump distance. As another example, once the jaws 801 are closed, the first pusher 76 can be advanced into the second jaw 80 to proximally move the shuttle 14 in direction 814b by the bump distance in the second jaw 80. The bump distance can be, for example, 1 mm to 6 mm, or more narrowly, from 1 mm to 4 mm, or more narrowly, from 1 mm to 3 mm, including every 1 mm increment within these ranges (e.g., 1 mm, 2 mm, 3 mm, 4 mm, 5 mm, 6 mm). FIG. 26C illustrates the proximal male stop 412 (e.g., the second male stop 412b) in the first female stop 416a before being bumped back by the bump distance, and FIG. 26D illustrates the proximal male stop 412 (e.g., the first male stop 412b) in the first female stop 416a after being bumped back the bump distance. When the second male stop 412b is in the position shown in FIG. 26D, the second pusher 86 can close the second male stop 412b (e.g., from the male stop first configuration to the male stop second configuration) and push the shuttle 14 to the second jaw 80.

FIGS. 26A-26D illustrate that clamping the jaws 801 against tissue can push the proximal male stop 412 away from the leading edge of the primary catch (e.g., the first female stop 416a) so that the pushers 803 can close the proximal male stop 412 and push the shuttle 14 out of the primary catch.

FIG. 26E illustrates an exemplary locked configuration of the first male stop 412a in the first female stop 416a in the first jaw 78. The locked configuration can be unlocked, for example, by clamping the jaws 801 against tissue, by advancing the second pusher 86 into the first jaw 78 to bump the shuttle 14 back by the bump distance, or by both.

FIG. 26F illustrates an exemplary locked configuration of the second male stop 412b in the first female stop 416a in the second jaw 80. The locked configuration can be unlocked, for example, by clamping the jaws 801 against tissue, by advancing the first pusher 76 into the second jaw 80 to bump the shuttle 14 back by the bump distance, or by both.

FIG. 26G illustrates an exemplary configuration after the first pusher 76 has pushed the first male stop 412a out of the first female stop 416a in the first jaw 78, for example, after being unlocked from or moved away from a locked configuration.

FIG. 26H illustrates an exemplary configuration after the second pusher 86 has pushed the second male stop 412b out of the first female stop 416a in the second jaw 80, for example, after being unlocked from or moved away from a locked configuration.

As another example, the proximal male stop 412 (e.g., the first male stop 412a) may not have a locked configuration in the first female stop 416a in the first jaw 78. In such variations, the first pusher 76 can close the proximal male stop 412 irrespective of the position of the position (e.g., longitudinal position) of the proximal male stop 412 in the first female stop 416a in the first jaw 78. For example, the first male stop 412a can be pushed out of the primary catch (e.g., the first female stop 416a in the first jaw 78) in any position, with or without pushback assistance. For example, without pushback assistance, the first male stop 412a can be pushed out of the primary catch in the first jaw 78 (e.g., the first female stop 416a in the first jaw 78) from any position in the primary catch, but with pushback assistance, the force necessary to push the first male stop 412a out of the primary catch can be reduced. Pushing (e.g., bumping) the shuttle 14 back in the first jaw 78 can get the shuttle 14 out of tension in the first jaw 78, or can relieve the tension of the shuttle 14 in the first jaw 78. When the shuttle 14 does not have an exposed tip for tissue to help with the bump back, the user can fire the opposite pusher first, and then fire the stitching pusher (e.g., the pusher that is in the same jaw as the shuttle 14). This can add an extra step to get the shuttle 14 out of tension, for example, out of a potential self-lock condition, and the user can then transit the shuttle 14 from the jaw with the stitching pusher to the opposite jaw.

As another example, the proximal male stop 412 (e.g., the second male stop 412b) may not have a locked configuration in the first female stop 416a in the second jaw 80. In such variations, the second pusher 86 can close the proximal male stop 412 irrespective of the position of the position (e.g., longitudinal position) of the proximal male stop 412 in the first female stop 416a in the second jaw 80. For example, the second male stop 412b can be pushed out of the primary catch (e.g., the first female stop 416a in the second jaw 80) in any position, with or without pushback assistance. For example, without pushback assistance, the second male stop 412b can be pushed out of the primary catch in the second jaw 80 (e.g., the first female stop 416a in the second jaw 80) from any position in the primary catch, but with pushback assistance, the force necessary to push the second male stop 412b out of the primary catch can be reduced. Pushing (e.g., bumping) the shuttle 14 back in the second jaw 80 can get the shuttle 14 out of tension in the second jaw 80, or can relieve the tension of the shuttle 14 in the second jaw 80. When the shuttle 14 does not have an exposed tip for tissue to help with the bump back, the user can fire the opposite pusher first, and then fire the stitching pusher (e.g., the pusher that is in the same jaw as the shuttle 14). This can add an extra step to get the shuttle 14 out of tension, for example, out of a potential self-lock condition, and the user can then transit the shuttle 14 from the jaw with the stitching pusher to the opposite jaw.

FIGS. 26A-26H illustrate that when the shuttle 14 is in the first jaw 78, the proximal male stop 412 can be the first male stop 412a and that when the shuttle 14 is in the second jaw 80, the proximal male stop 412 can be the second male stop 412b.

FIGS. 26A-26E illustrate that the male stops 412 can be disengaged from the female stops 416 by advancing the pushers 803. For example, the male stops 412 can be closed by advancing the pushers. The lateral male stops (e.g., stops 412_{L} can be active stops and the radial male stops (e.g., stops 412_{R}) can be passive stops.

FIGS. 27A and 27B illustrate that the jaws 801 can have one tissue guide 962, and that the tissue guide 962 can extend from the second jaw 80. FIGS. 27A and 27B illustrate that the tissue guide 962 can extend proximally away from the second jaw 80. The tissue guide 962 can be offset from the device longitudinal axis 956. For example, FIGS. 27A and 27B illustrate that the a first lateral side of the second jaw 80 can have the tissue guide 962 and that a second lateral side of the second jaw 80 may not have a tissue guide 962, or vice versa. The single tissue guide 962 in FIGS. 27A and 27B can provide the same benefits as the two tissue guides described above and shown, for example, in FIGS. 1, 14F, 14G, and elsewhere.

FIGS. 28A and 28B illustrate that the shuttle first and second tips 164a, 164b can have a tapered surface 976. The tapered surface 976 can help make the leading tip less proud as the shuttle 14 is loaded into the jaws 801 or as the shuttle 14 is passed between the jaws 801, for example, by relieving the leading edge from about 0.002 inches to about 0.008 inches, including every 0.001 inch increment within this range (e.g., 0.002 inches, 0.005 inches, 0.008 inches). This can make loading the shuttle 14 into the jaws 801 easier, for example, as compared to the same shuttle 14 without the tapered surface 976, can making passing the shuttle 14 between the jaws 801 easier, for example, as compared to the same shuttle 14 without the tapered surface 976, can make piercing tissue easier, for example, as compared to the same shuttle 14 without the tapered surface 976, or any combination thereof. The terminal tip 165 of the shuttle first and second tips 164a, 164b can have the distal terminal edge shown, for example, so that the pushers 803 are inhibited or prevented from slipping or subducting under the shuttle first and second tips 164a, 164b as the pushers 803 are advanced.

FIG. 28A illustrates that the first male stop 412a can have a hammerhead shape. For example, FIG. 28A illustrates that the first male stop 412a can have a wide end that tapers toward a narrow end. FIG. 28A illustrates that the narrow end can be closer to the terminal tip 165 of the shuttle first tip 164a than the wide end.

FIG. 28B illustrates that the second male stop 412b can have a hammerhead shape. For example, FIG. 28B illustrates that the second male stop 412b can have a wide end that tapers toward a narrow end. FIG. 28B illustrates that the narrow end can be closer to the terminal tip 165 of the shuttle second tip 164b than the wide end.

FIGS. 28A and 28B illustrate that the shuttle 14 can have a length of about 0.357 inches. As another example, the shuttle 14 can have a length of about 0.297 inches.

FIGS. 29A and 29B illustrate that the pushers 803 can have overstroke, for example, that each of the pushers 803 can be advanced out of their respective jaws 801. For example, FIG. 29A illustrates that first pusher 76 can be advanced out of the first jaw 78, and FIG. 29B illustrates that the second pusher 86 can be advanced out of the second jaw 80.

FIG. 29A illustrates, for example, that when the selector 806 is in the top position and the second trigger 804 is actuated (e.g., forward), the first pusher 76 can come out of the first jaw 78 and into the opposite jaw (e.g., the second jaw 80).

FIG. 29B illustrates, for example, that when the selector 806 is in the bottom position and the second trigger 804 is actuated (e.g., forward), the second pusher 86 can come out of the second jaw 80 and into the opposite jaw (e.g., the first jaw 78).

FIGS. 29A and 29B illustrate that the distal tips of the pushers 803 can travel past the zero point. The zero point can be the terminal end of the first and second jaw tracks 64, 66. The zero point can be the terminal distal ends of the jaws 801.

FIG. 29A illustrates, for example, that the distal tip of the first pusher 76 can travel past the tip of the first jaw 78 and into the second jaw 80 (e.g., when the jaws 801 are closed) so that the second male stop 412b of the shuttle 14 can be pushed past the distal edge of the first female stop 416a in the second jaw 80. The overstroke of the first pusher 76 can advance the first pusher 76 into the second jaw 80 to bump the shuttle 14 proximally into the second jaw 80 by the bump distance. The overstroke distance of the first pusher 76 can be the same as the bump distance. As another example, the overstroke distance of the first pusher 76 can be the same as or greater than the bump distance. For example, the overstroke distance of the first pusher 76 can be from about 1 mm to 20 mm, or more narrowly, from 1 mm to 10 mm, or more narrowly, from 1 mm to 6 mm, or more narrowly, from 1 mm to 3 mm, including every 1 mm increment within these ranges (e.g., 1 mm, 2 mm, 3 mm, 4 mm, 5 mm, 6 mm, 10 mm, 20 mm). The overstroke distance of the first pusher 76 can be the length of the portion of the first pusher 76 that is outside of the first jaw 78. The overstroke distance of the first pusher 76 can be the length of the portion of the first pusher 76 that is inside of the second jaw 80 when the jaws 801 are fully closed and the first pusher 76 is fully advanced by the second trigger 804.

FIG. 29B illustrates, for example, that the distal tip of the second pusher 86 can travel past the tip of the second jaw 80 and into the first jaw 78 (e.g., when the jaws 801 are closed) so that the first male stop 412a of the shuttle 14 can be pushed past the distal edge of the first female stop 416a in the first jaw 78. The overstroke of the second pusher 86 can advance the second pusher 86 into the first jaw 78 to bump the shuttle 14 proximally into the first jaw 78 by the bump distance. The overstroke distance of the second pusher 86 can be the same as the bump distance. For example, the overstroke distance of the second pusher 86 can be from about 1 mm to 20 mm, or more narrowly, from 1 mm to 10 mm, or more narrowly, from 1 mm to 6 mm, or more narrowly, from 1 mm to 3 mm, including every 1 mm increment within these ranges (e.g., 1 mm, 2 mm, 3 mm, 4 mm, 5 mm, 6 mm, 10 mm, 20 mm). The overstroke distance of the second pusher 86 can be the length of the portion of the second pusher 86 that is outside of the second jaw 80. The overstroke distance of the second pusher 86 can be the length of the portion of the second pusher 86 that is inside of the first jaw 78 when the jaws 801 are fully closed and the second pusher 86 is fully advanced by the second trigger 804.

The overstroke distance of the first and second pushers 76, 86 can be the same as or different from each other. For example, FIGS. 29A and 29B illustrate that the overstroke distance of the first and second pushers 76, 86 can be the same as each other.

The jaws 801 are shown in a fully open configuration in FIGS. 29A and 29B so that the overstroke of the pushers 803 can be seen. The shuttle 14 in FIGS. 29A and 29B is shown transparent for illustrative purposes only.

FIGS. 29A and 29B illustrate that the pushers 803 can be advanceable out of the jaws 801 much further than they would need to push the shuttle 14 into the opposite jaw so that the shuttle 14 can be pushed past the finish line. The jaws 801 can each have a finish line. The finish line in the first jaw 78 can be, for example, the distal edge of the first female stop 416a in the first jaw 78. The finish line in the first jaw 78 can be, for example, the point at which the first male stop 412a becomes engaged with the first female stop 416a in the first jaw 78. The finish line in the second jaw 80 can be, for example, the distal edge of the first female stop 416a in the second jaw 80. The finish line in the second jaw 80 can be, for example, the point at which the second male stop 412b becomes engaged with the first female stop 416a in the second jaw 80.

The overstroke shown in FIGS. 29A and 29B can inhibit or prevent the male stops 412 getting locked in female stops 416. For example, FIG. 29A illustrates that the overstroke of the first pusher 76 can inhibit or prevent the second male stop 412b of the shuttle 14 (e.g., on the shuttle second tip 164b) from getting locked in the first female stop 416a in the second jaw 80, for example, by bumping the shuttle 14 proximally along the second track 66 in the second jaw 80 by the bump distance. As another example, FIG. 29B illustrates that the overstroke of the second pusher 86 can inhibit or prevent the first male stop 412a of the shuttle 14 (e.g., on the shuttle first tip 164a) from getting locked in the first female stop 416a in the first jaw 78, for example, by bumping the shuttle 14 proximally along the first shuttle track 64 in the first jaw 78 by the bump distance.

The proximal movement of the shuttle 14 caused by the overstroke of the first pusher 76 can be a preparatory movement before the second pusher 86 is advanced.

The proximal movement of the shuttle 14 caused by the overstroke of the second pusher 86 can be a preparatory movement before the first pusher 76 is advanced.

The overstroke of the pushers 803 can inhibit or prevent the shuttle 14 from getting into a locked configuration with a female stop 416.

The pushers 803 can have a neutral position of about 0.10 inches to about 0.30 inches behind the shuttle first and second tips 164a, 164b, including every 0.01 inch increment within this range (e.g., 0.10 inches, 0.20 inches, 0.30 inches). For example, when the first pusher 76 pushes the shuttle 14 fully into the second jaw 80 and the second pusher 86 is fully retracted, the distal terminal end of the second pusher 86 can be about 0.10 inches to about 0.30 inches proximal the shuttle second tip 164b. As another example, when the second pusher 86 pushes the shuttle 14 fully into the first jaw 78 and the first pusher 76 is fully retracted, the distal terminal end of the first pusher 76 can be about 0.10 inches to about 0.30 inches proximal the shuttle first tip 164a.

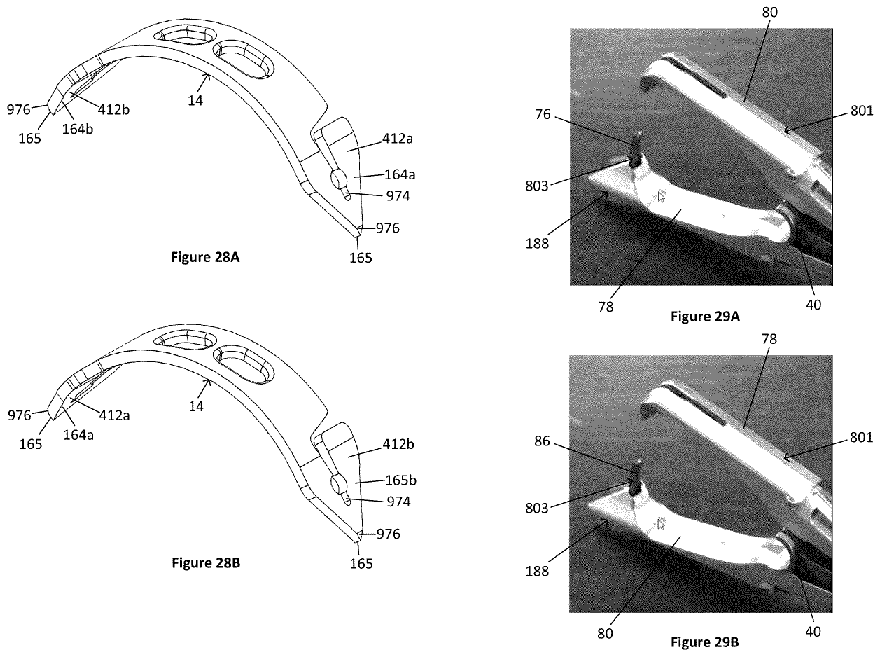

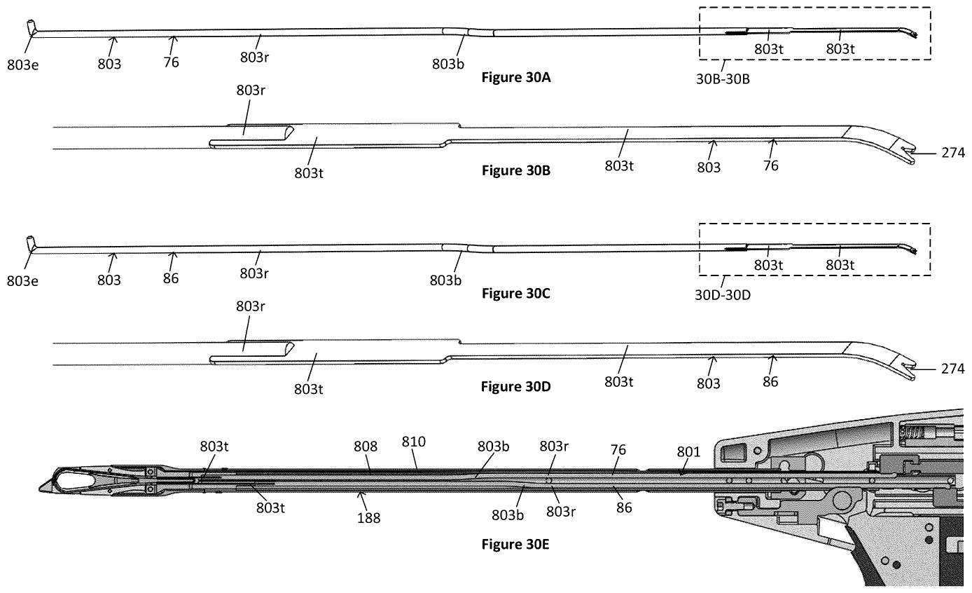

FIGS. 30A-30E illustrate variations of the pushers 803. FIG. 30E illustrates that the pushers 803 can have one or multiple bends 803b when in a retracted position in the inner tube 808. The bends 803b are also shown in FIGS. 30A and 30C. The pushers 803 can always have a bend (e.g., the bends 803b shown in FIGS. 30A-30E). As another example, the pushers 803 can have one or more bends (e.g., the bends 803b shown in FIGS. 30A-30E) when the pushers 803 are advanced by the second trigger 804. The pins 828 can inhibit or prevent the bends 803b from bending any further. FIGS. 30A-30E illustrate that the first and second pushers 76, 86 can be bendable and can take on a curved configuration, for example, when in one or both of the jaw tracks 64 and/or 66. FIGS. 30A-30E illustrate that the first and second pushers 76, 86 can have the shuttle seats 274 as shown. The shuttle seats 274 can be a male or female shape and inverse to the shape of the shuttle tips 164.

The device 188 can have the dimensions shown in FIGS. 31A-31D. The dimensions shown in FIGS. 31A-31D can be in inches. The dimensions shown in FIGS. 31A-31D are exemplary.

FIG. 31A illustrates that the device 188 can be inserted into a lumen 978 of a cannula 980, for example, to be deployed percutaneously through the cannula 980 inserted in a patient at a target site. The lumen 978 can have a diameter, for example, from about 2 mm to about 20 mm, including every 1 mm increment within this range (e.g., 2 mm, 5 mm, 20 mm).

FIGS. 31A-31C illustrate that the jaws 801 can have one tissue guide 962 as shown.

FIG. 31D illustrates that the jaws 801 may not have a tissue guide 962.

FIGS. 31A and 31E illustrate that the grip 799, the second trigger 804, and the pin 862 can have the arrangement shown. Half of the grip 799 and the second trigger 804 are shown transparent in FIG. 31E so that the pin 862 can be seen. This arrangement of features can, for example, ergonomically provide a better feel for the users.

FIGS. 31A and 31F₁-31F₃ illustrate that the lockout bar 888 can have a lockout bar length 888_{L} that can be adjustable. Adjusting the length of the lockout bar can alter the travel distance (e.g., the linear travel distance in direction 814a) of the of the second trigger 804, which can change the travel distance (e.g., the linear travel distance in direction 814a) of the pushers 803. By adjusting the length of the lockout bar 888, the stroke distance of the second trigger 804 can be adjusted. This can be important for tuning and overall device reliability so that the distance that the pushers 803 can be advanced by the second trigger 804 can be adjusted.

FIGS. 31A and 31F₁-31F₃ illustrate that the lockout bar 888 can be expandable and contractible so that the lockout bar length 888_{L} can be adjustable. FIGS. 31A and 31F₁-31F₃ illustrate, for example, that the lockout bar 888 can have a lockout bar first portion 888a and a lockout bar second portion 888b. The lockout bar first and second portions 888a, 888b can be movable relative to each other to expand and contract the lockout bar 888. For example, FIGS. 31A and 31F₁-31F₃ illustrate that the lockout bar first portion 888a can have a boss 982 that can be adjustably positionable in a recess 984 in the lockout bar second portion 888b. The boss 982 and the recess 984 can be threaded so that the boss and the recess 984 can be in threaded engagement with each other. The lockout bar length 888_{L} can be increased and decreased, for example, by twisting (e.g., screwing and unscrewing) the lockout first portion 888a relative to the lockout second portion 888b.

FIG. 31F₁-31F₃ illustrate that to shorten the lockout bar 888, the lockout first portion 888a can be movable toward the lockout second portion 888b or vice versa. Shortening the lockout bar length 888_{L} can decrease the travel distance (e.g., the linear travel distance in direction 814a) of the of the second trigger 804, which can decrease the travel distance (e.g., the linear travel distance in direction 814a) of the pushers 803.

FIG. 31F₁-31F₃ illustrate that to lengthen the lockout bar 888, the lockout bar first portion 888a can be movable away from the lockout bar second portion 888b or vice versa. Lengthening the lockout bar length 888_{L} can increase the travel distance (e.g., the linear travel distance in direction 814a) of the of the second trigger 804, which can increase the travel distance (e.g., the linear travel distance in direction 814a) of the pushers 803.

FIG. 31F₁-31F₃ illustrate that the lockout bar 888 can have a lockout bar first length 888_{L1}, a lockout bar second length 888_{L2}, and a lockout bar third length 888_{L3}, respectively. The lockout bar 888 can be adjustable between these lengths. The lockout bar first length 888_{L1} can be the shortest length of the lockout bar 888 (e.g., when the lockout bar 888 is in a fully contracted configuration), the lockout bar third length 888_{L3} can be the longest length of the lockout bar 888 (e.g., when the lockout bar 888 is in a fully expanded configuration), and the lockout bar second length 888_{L2} can be any length of the lockout bar 888 between the lockout bar first and third lengths 888_{L1}, 888_{L3}.

The difference in length between the lockout bar first and third lengths 888_{L1}, 888_{L3} can be, for example, 0.5 mm to 10.0 mm, or more narrowly, 0.5 mm to 5.0 mm, including every 0.1 mm increment within these ranges (e.g., 0.5 mm, 5.0 mm, 10.0 mm). FIG. 31F₁ thus shows that when the lockout bar 888 is in a fully contracted configuration, the lockout bar length 888_{L} can be increased by 0.5 mm to 10.0 mm, or more narrowly, 0.5 mm to 5.0 mm, including every 0.1 mm increment within these ranges (e.g., 0.5 mm, 5.0 mm, 10.0 mm), and FIG. 31F₃ thus shows that when the lockout bar 888 is in a fully expanded configuration, the lockout bar length 888_{L} can be decreased by 0.5 mm to 10.0 mm, or more narrowly, 0.5 mm to 5.0 mm, including every 0.1 mm increment within these ranges (e.g., 0.5 mm, 5.0 mm, 10.0 mm).

The amount by which the lockout bar length 888_{L} is changed can result in the same change in the travel distance (e.g., the linear travel distance in direction 814a) of the of the second trigger 804 and can result in the same change in the travel distance (e.g., the linear travel distance in direction 814a) of the of the pushers 803. For example, if the lockout bar length 888_{L} is increased by 2.0 mm, the travel distance (e.g., the linear travel distance in direction 814a) of the of the second trigger 804 can be increased by 2.0 mm and the travel distance (e.g., the linear travel distance in direction 814a) of the of the pushers 803 can be increased by 2.0 mm. As another example, if the lockout bar length 888_{L} is decreased by 2.0 mm, the travel distance (e.g., the linear travel distance in direction 814a) of the of the second trigger 804 can be decreased by 2.0 mm and the travel distance (e.g., the linear travel distance in direction 814a) of the of the pushers 803 can be decreased by 2.0 mm.

The lockout bar length 888_{L} can be, for example, 10.0 mm to 40.0 mm, or more narrowly, 18.0 mm to 28.0 mm, including every 1.0 mm increment within these ranges (e.g., 10.0 mm, 18.0 mm, 25.0 mm, 28.0 mm, 40.0 mm).

The lockout bar length 888_{L} can be adjusted during assembly, after assembly, or both during assembly and after assembly.

The features in FIGS. 1-31F₃, the features described herein, and/or the features contemplated herein can be combined with each other in any combination. For example, the device 188 in FIG. 1 can have any combination of the various shuttle features, the various jaw features, and/or of the various shuttle retention features (e.g., the male and female stops 412, 416) disclosed herein.

For example, a suture passer is disclosed that can have a first jaw (e.g., the jaw 78 or the jaw 80). The first jaw can have a first jaw channel (e.g., the track 64 or the track 66) and a first jaw stop (e.g., a male stop 412 or a female stop 416). The suture passer can have a shuttle (e.g., the shuttle 14). The shuttle can have a shuttle longitudinal axis. The shuttle can have a shuttle first stop (e.g., a male stop 412 or a female stop 416). The first jaw stop can be a female stop and the shuttle first stop can be a male stop, or vice versa. The shuttle can be slidable in the first jaw channel. The shuttle first stop can extend away from the shuttle longitudinal axis. The shuttle first stop can be releasably engageable with the first jaw stop. The shuttle first stop can be movable into and out of the first jaw stop. The shuttle first stop can have a shuttle first stop first end and a shuttle first stop second end. The shuttle first stop second end can be farther from a center longitudinal axis of the shuttle than the shuttle first stop first end. The shuttle first stop second end can be closer to a longitudinal center of the shuttle than the shuttle first stop first end. The shuttle first stop first end can be narrower than the shuttle first stop second end. The shuttle first stop second end can be releasably engageable with the first jaw stop. The shuttle first stop second end can be movable into and out of the first jaw stop. The shuttle first stop can be engageable with the first jaw stop. When the shuttle first stop is engaged with the first jaw stop, the shuttle first stop can be in the first jaw stop. When the shuttle first stop is engaged with the first jaw stop, the shuttle first stop second end can be in the first jaw stop. When the shuttle first stop is engaged with the first jaw stop, the shuttle can be passively retained in the first jaw. When the shuttle first stop is in the first jaw stop, movement of the shuttle in the first jaw channel can be inhibited. When the shuttle first stop is engaged with the first jaw stop, the shuttle first stop can have a shuttle first stop first configuration (e.g., the male stop first configuration). When the shuttle first stop is not engaged with the first jaw stop (e.g., when the shuttle first stop is between the first jaw stop and a tip of the first jaw), the shuttle first stop can have a shuttle first stop second configuration (e.g., the male stop second configuration). The shuttle first stop can be less deflected when in the shuttle first stop first configuration than when in the shuttle first stop second configuration. The shuttle first stop first configuration can be a non-deflected configuration of the shuttle first stop. The shuttle first stop second configuration can be a deflected configuration of the shuttle first stop. The shuttle first stop can be deflectable into and out of the first jaw stop.

The suture passer can have a second jaw (e.g., the jaw 78 or the jaw 80). The second jaw can have a second jaw channel (e.g., the track 64 or the track 66) and a second jaw stop (e.g., a male stop 412 or a female stop 416). The shuttle can have a shuttle second stop (e.g., a male stop 412 or a female stop 416). The second jaw stop can be a female stop and the shuttle second stop can be a male stop, or vice versa. The shuttle can be slidable in the second jaw channel. The shuttle second stop can extend away from the shuttle longitudinal axis. The shuttle second stop can be releasably engageable with the second jaw stop. The shuttle second stop can be movable into and out of the second jaw stop. The shuttle second stop can have a shuttle second stop first end and a shuttle second stop second end. The shuttle second stop second end can be farther from a center longitudinal axis of the shuttle than the shuttle second stop first end. The shuttle second stop second end can be closer to a longitudinal center of the shuttle than the shuttle second stop first end. The shuttle second stop first end can be narrower than the shuttle second stop second end. The shuttle second stop second end can be releasably engageable with the second jaw stop. The shuttle second stop second end can be movable into and out of the second jaw stop. The shuttle second stop can be engageable with the second jaw stop. When the shuttle second stop is engaged with the second jaw stop, the shuttle second stop can be in the second jaw stop. When the shuttle second stop is engaged with the second jaw stop, the shuttle second stop second end can be in the second jaw stop. When the shuttle second stop is engaged with the second jaw stop, the shuttle can be passively retained in the second jaw. When the shuttle second stop is in the second jaw stop, movement of the shuttle in the second jaw channel can be inhibited. When the shuttle second stop is engaged with the second jaw stop, the shuttle second stop can have a shuttle second stop first configuration (e.g., the male stop first configuration). When the shuttle second stop is not engaged with the second jaw stop (e.g., when the shuttle second stop is between the second jaw stop and a tip of the second jaw), the shuttle second stop can have a shuttle second stop second configuration (e.g., the male stop second configuration). The shuttle second stop can be less deflected when in the shuttle second stop first configuration than when in the shuttle second stop second configuration. The shuttle second stop first configuration can be a non-deflected configuration of the shuttle second stop. The shuttle second stop second configuration can be a deflected configuration of the shuttle second stop. The shuttle second stop can be deflectable into and out of the second jaw stop.

At least one of the shuttle first stop and the shuttle second stop can be a male stop.

At least one of the shuttle first stop and the shuttle second stop can have a hammerhead shape.

The shuttle first stop can extend radially away from the shuttle longitudinal axis, and the shuttle second stop can extend radially away from the shuttle longitudinal axis.

The shuttle first stop can extend radially away from the shuttle longitudinal axis, and the shuttle second stop can extend laterally away from the shuttle longitudinal axis.

The shuttle first stop can extend laterally away from the shuttle longitudinal axis, and the shuttle second stop can extend laterally away from the shuttle longitudinal axis.

The shuttle first stop can be a radial male stop (e.g., 412_{R}) or a lateral male stop (e.g., 412_{L}). The shuttle first stop can be a radial female stop (e.g., 416_{R}) or a lateral female stop (e.g., 416_{L}). The shuttle second stop can be a radial male stop (e.g., 412_{R}) or a lateral male stop (e.g., 412_{L}). The shuttle second stop can be a radial female stop (e.g., 416_{R}) or a lateral female stop (e.g., 416_{L}).

The first jaw stop can be a radial male stop (e.g., 412_{R}) or a lateral male stop (e.g., 412_{L}). The first jaw stop can be a radial female stop (e.g., 416_{R}) or a lateral female stop (e.g., 416_{L}). The second jaw stop can be a radial male stop (e.g., 412_{R}) or a lateral male stop (e.g., 412_{L}). The second jaw stop can be a radial female stop (e.g., 416_{R}) or a lateral female stop (e.g., 416_{L}).

Before the shuttle is loaded into the first jaw channel, the shuttle can have a first radius of curvature. After the shuttle is loaded into the first jaw channel, the shuttle can have a second radius of curvature. The second radius of curvature can be less than the first radius of curvature. The shuttle can be biased to have the first radius of curvature.

A method of suturing tissue with a suture passer (e.g., with the device 188) is disclosed. The suture passer can have a first jaw (e.g., the first jaw 78), a second jaw (e.g., the second jaw 80), a first trigger (e.g., the first trigger 802), a second trigger (e.g., the second trigger 804), and a selector (e.g., the selector 806). The method can include selecting a first pusher (e.g., the first pusher 76) with the selector. The method can include moving the first jaw toward the second jaw with the first trigger. The method can include moving the first pusher from a first pusher first position to a first pusher second position with the second trigger to move the shuttle from the first jaw to the second jaw. The method can include moving the first pusher from the first pusher second position to the first pusher first position with the second trigger. The method can include moving the first jaw away from the second jaw with the first trigger.

Selecting the first pusher with the selector can include aligning a mover (e.g., the mover 840) with a first engager (e.g., the first engager 847a). The mover can be connected to the second trigger. The first engager can be connected to the first pusher.

Moving the first pusher from the first pusher first position to the first pusher second position can include advancing the first engager with the mover via the second trigger.

Moving the first pusher from the first pusher second position to the first pusher first position can include retracting the first engager with the mover via the second trigger.

Moving the first jaw away from the second jaw with the first trigger can include moving an opener (e.g., the opener 40) between the first jaw and the second jaw away from a distal end of the first jaw and the second jaw. The opener can be connected to the first trigger.

Moving the first jaw toward the second jaw with the first trigger can include moving a closer (e.g., the closer 34) over the first jaw and the second jaw toward a distal end of the first jaw and the second jaw. The closer can be connected to the first trigger.

The method can include selecting a second pusher (e.g., the second pusher 86) with the selector. The method can include moving the first jaw toward the second jaw with the first trigger. The method can include moving the second pusher from a second pusher first position to a second pusher second position with the second trigger to move the shuttle from the second jaw to the first jaw. The method can include moving the second pusher from the second pusher second position to the second pusher first position with the second trigger. The method can include moving the first jaw away from the second jaw with the first trigger.

Selecting the first pusher with the selector can include aligning a mover (e.g., the mover 840) with a first engager (e.g., the first engager 847a). Moving the first pusher from the first pusher first position to the first pusher second position can include advancing the first engager with the mover via the second trigger. Selecting the second pusher with the selector can include aligning the mover with a second engager (e.g., the second engager 847b). Moving the second pusher from the second pusher first position to the second pusher second position can include advancing the second engager with the mover via the second trigger. The mover can be connected to the second trigger. The first engager can be connected to the first pusher. The second engager can be connected to the second pusher.

Moving the first pusher from the first pusher second position to the first pusher first position can include retracting the first engager with the mover via the second trigger. Moving the second pusher from the second pusher second position to the second pusher first position can include retracting the second engager with the mover via the second trigger.

Moving the first pusher from the first pusher first position to the first pusher second position can include advancing the first pusher. Moving the second pusher from the second pusher first position to the second pusher second position can include advancing the second pusher.

Moving the first pusher from the first pusher second position to the first pusher first position can include retracting the first pusher. Moving the second pusher from the second pusher second position to the second pusher first position can include retracting the second pusher.

Moving the first jaw toward the second jaw with the first trigger can include unlocking the second trigger.

Unlocking the second trigger can include moving a lockout gate (e.g., the lockout gate 886) from a lockout gate first position to a lockout gate second position with the first trigger. When the lockout gate is in the lockout gate first position, movement of the second trigger can be inhibited by the lockout gate. When the lockout gate is in the lockout gate second position, the second trigger can be movable to move the first pusher or the second pusher.

A suture passer (e.g., the device 188) is disclosed. The suture passer can have a mover (e.g., the mover 840). The mover can be movable. The mover can be blockable and unblockable, for example, via the first trigger 802, the second trigger 804, and/or the selector 806. The mover can be blockable and unblockable with one or multiple barriers (e.g., two barriers). The mover can be blockable, for example, by a first barrier (e.g., the lockout gate 886) and/or by a second barrier (e.g., the extension 912). The first barrier can be movable, for example, relative to the mover. The second barrier can be non-movable. The second barrier can be fixed. The mover can move relative to the first barrier. The mover can move relative to the second barrier. The mover can be lockable and unlockable, for example, via the first trigger 802, the second trigger 804, and/or the selector 806. The first jaw can have a first jaw track (e.g., the track 64) and a first jaw first stop (e.g., a female stop 416). The suture passer can have a shuttle (e.g., the shuttle 14). The shuttle can have a shuttle longitudinal axis and a shuttle first stop (e.g., a male stop 412). The shuttle can be movable in the first jaw track via the mover. The shuttle first stop can extend away from the shuttle longitudinal axis. The shuttle first stop can be movable into and out of the first jaw first stop. When the shuttle first stop is in the first jaw first stop, the shuttle can be retained in the first jaw. When the shuttle first stop is in the first jaw first stop, the shuttle can be passively retained in the first jaw. The shuttle can be retainable in the first jaw when the shuttle first stop is in the first jaw first stop. The suture passer may not have the selector. For example, for variations in which the device 188 has one pusher or for variations in which the pushers 803 of the device are movable in unison (e.g., for variations in which the pushers 803 are not independently movable), the suture passer may not have the selector. The mover can have a mover first position and a mover second position. The mover first position can be a mover first longitudinal position and the mover second position can be a mover second longitudinal position. The mover can have a mover first longitudinal position and a mover second longitudinal position. The mover can be movable from the mover first longitudinal position to the mover second longitudinal position and vice versa. The mover can be releasably lockable in the mover first longitudinal position. The mover can be farther from the first jaw track when in the mover first longitudinal position than when in the mover second longitudinal position. The mover first longitudinal position can be a mover retracted position. The mover second longitudinal position can be a mover advanced position. The mover can be lockable in the mover first longitudinal position. The mover can be longitudinally lockable in the mover first longitudinal position. The mover can be unlockable from the mover first longitudinal position via the first trigger. The mover can be unlockable from the mover first longitudinal position by moving a barrier (e.g., the lockout gate 886). The mover can be unlockable from the mover first longitudinal position by moving the first barrier (e.g., the lockout gate 886). The barrier can be moved via the first trigger. When the mover is in a locked configuration and in the mover first longitudinal position, the mover can be inhibited or prevented from moving toward the mover second longitudinal position. When the mover is in an unlocked configuration and in the mover first longitudinal position, the mover can be movable toward the mover second longitudinal position. The mover can have a mover third position and a mover fourth position. The mover third position can be a mover first transverse position and the mover fourth position can be a mover second transverse position. The mover can have a mover first transverse position and a mover second transverse position. The mover can be movable from the mover first transverse position to the mover second transverse position and vice versa. The mover can be releasably lockable in the mover first transverse position. The mover can be releasably lockable in the mover second transverse position. The mover can be closer to the first jaw track when in the mover first transverse position than when in the mover second transverse position. The mover first transverse position can be a mover raised position. The mover second transverse position can be a mover lowered position. When the mover is in the mover first longitudinal position, the mover can be movable from the mover first transverse position to the mover second transverse position and vice versa. When the mover is in the mover second longitudinal position, the mover can be inhibited or prevented from moving from the mover first transverse position to the mover second transverse position and vice versa. The mover can be lockable in the mover first transverse position. When the mover is in the mover first transverse position, the mover can be lockable in the mover first transverse position by moving the mover from the mover first longitudinal position to the mover second longitudinal position. When the mover is in the mover second longitudinal position and in the mover first transverse position, the mover can be inhibited or prevented from moving toward the mover second transverse position. When the mover is in the mover second longitudinal position and in the mover first transverse position, a barrier (e.g., the extension 912) between the mover first and second transverse positions can inhibit or prevent the mover from moving toward the mover second transverse position. When the mover is in the mover second longitudinal position and in the mover first transverse position, the second barrier (e.g., the extension 912) between the mover first and second transverse positions can inhibit or prevent the mover from moving toward the mover second transverse position. The mover can be unlockable from the mover first transverse position. When the mover is in the mover first transverse position, the mover can be unlockable from the mover first transverse position by moving the mover from the mover second longitudinal position to the mover first longitudinal position. When the mover is in the mover first longitudinal position and in the mover first transverse position, the mover can be movable toward the mover second transverse position.

The suture passer can have a second jaw (e.g., the second jaw 80). The second jaw can have a second jaw track (e.g., the track 66) and a second jaw first stop (e.g., a female stop 416). The shuttle can be movable in the second jaw track. The shuttle second stop can extend away from the shuttle longitudinal axis. The shuttle second stop can be movable into and out of the second jaw first stop. When the shuttle second stop is in the second jaw first stop, the shuttle can be retained in the second jaw. When the shuttle second stop is in the second jaw first stop, the shuttle can be passively retained in the second jaw. The shuttle can be retainable in the second jaw when the shuttle second stop is in the second jaw first stop. The mover can be lockable in the mover second transverse position. When the mover is in the mover second transverse position, the mover can be lockable in the mover second transverse position by moving the mover from the mover first longitudinal position to the mover second longitudinal position. When the mover is in the mover second longitudinal position and in the mover second transverse position, the mover can be inhibited or prevented from moving toward the mover first transverse position. When the mover is in the mover second longitudinal position and in the mover second transverse position, a barrier (e.g., the extension 912) between the mover first and second transverse positions can inhibit or prevent the mover from moving toward the mover first transverse position. The mover can be unlockable from the mover second transverse position. When the mover is in the mover second transverse position, the mover can be unlockable from the mover second transverse position by moving the mover from the mover second longitudinal position to the mover first longitudinal position. When the mover is in the mover first longitudinal position and in the mover second transverse position, the mover can be movable toward the mover first transverse position. The mover can be lockable and unlockable from the mover first and/or second transverse positions via a second trigger (e.g., the second trigger 804).

The suture passer can have a first jaw second stop and a second jaw second stop. The shuttle first stop can be movable into and out of the first jaw second stop. The shuttle second stop can be movable into and out of the second jaw second stop. The first jaw second stop can be closer to a tip of the first jaw than the first jaw first stop. The second jaw second stop can be closer to a tip of the second jaw than the second jaw first stop.

When the shuttle first stop is in the first jaw second stop, the shuttle can be passively retainable in the first jaw. When the shuttle first stop is in the first jaw second stop, the shuttle can be passively retained in the first jaw. When the shuttle second stop is in the second jaw second stop, the shuttle can be passively retainable in the second jaw. When the shuttle second stop is in the second jaw second stop, the shuttle can be passively retained in the second jaw

The first jaw first stop and the second jaw first stop can be primary shuttle catches. The first jaw second stop and the second jaw second stop can be secondary shuttle catches.

The shuttle first stop can extend laterally from the shuttle longitudinal axis. The shuttle second stop can extend laterally from the shuttle longitudinal axis. The first jaw first stop can extend laterally from the first jaw track. The first jaw second stop can extend laterally from the first jaw track. The second jaw first stop can extend laterally from the second jaw track. The second jaw second stop can extend laterally from the second jaw track.

The first jaw first stop and the first jaw second stop can be, for example, a first female stop 416a and a second female stop 416b, respectively. The first jaw first stop and the first jaw second stop can be, for example, the first female stop 416a and the second female stop 416b shown in FIGS. 24A and 24B. The second jaw first stop and the second jaw second stop can be, for example, a first female stop 416a and a second female stop 416b, respectively. The second jaw first stop and the second jaw second stop can be, for example, the first female stop 416a and the second female stop 416b shown in FIGS. 24C and 24D.

A suture passer (e.g., the device 188) is disclosed. The suture passer can have a first jaw (e.g., the first jaw 78) and a shuttle (e.g., the shuttle 14). The first jaw can have a first jaw track (e.g., the track 64), a first jaw first stop, and a first jaw second stop. The shuttle can have a shuttle longitudinal axis and a shuttle first stop. The shuttle can be movable in the first jaw track. The shuttle first stop and the first jaw first stop can be engageable with each other. The shuttle first stop and the first jaw second stop can be engageable with each other. When the shuttle first stop and the first jaw first stop are engaged with each other, the shuttle can be retainable in the first jaw. When the shuttle first stop and the first jaw first stop are engaged with each other, the shuttle can be passively retained in the first jaw. The shuttle can be passively retainable in the first jaw when the shuttle first stop and the first jaw first stop are engaged with each other. When the shuttle first stop and the first jaw second stop are engaged with each other, the shuttle can be retainable in the first jaw. When the shuttle first stop and the first jaw second stop are engaged with each other, the shuttle can be passively retained in the first jaw. The shuttle can be passively retainable in the first jaw when the shuttle first stop and the first jaw second stop are engaged with each other. The first jaw first stop can be a female stop (e.g., a female stop 416), the first jaw second stop can be a female stop (e.g., a female stop 416), and the shuttle first stop can be a male stop (e.g., a male stop 412). The first jaw first stop can be a male stop (e.g., a male stop 412), the first jaw second stop can be a male stop (e.g., a male stop 412), and the shuttle first stop can be a female stop (e.g., a female stop 416).

A suture passer (e.g., the device 188) is disclosed. The suture passer can have a first jaw (e.g., the first jaw 78) and a shuttle (e.g., the shuttle 14). The first jaw can have a first jaw track (e.g., the track 64), a first jaw first stop (e.g., a first female stop 416a), and a first jaw second stop (e.g., a second female stop 416b). The shuttle can have a shuttle longitudinal axis and a shuttle first stop (e.g., a first male stop 412a). The shuttle can be movable in the first jaw track. The shuttle first stop can be movable into and out of the first jaw first stop. The shuttle first stop can be movable into and out of the first jaw second stop. The shuttle can be retainable in the first jaw when the shuttle first stop is in the first jaw first stop. The shuttle can be passively retainable in the first jaw when the shuttle first stop is in the first jaw first stop. When the shuttle first stop is in the first jaw first stop, the shuttle can be passively retained in the first jaw. The shuttle can be retainable in the first jaw when the shuttle first stop is in the first jaw second stop. The shuttle can be passively retainable in the first jaw when the shuttle first stop is in the first jaw second stop. When the shuttle first stop is in the first jaw second stop, the shuttle can be passively retained in the first jaw.

The first jaw first stop can be farther from a tip of the first jaw than the first jaw second stop.

The suture passer can have a second jaw (e.g., the second jaw 80). The second jaw can have a second jaw track (e.g., the track 66), a second jaw first stop, and a second jaw second stop. The shuttle can be movable in the second jaw track. The shuttle can have a shuttle second stop. The shuttle second stop and the second jaw first stop can be engageable with each other. The shuttle second stop and the second jaw second stop can be engageable with each other. When the shuttle second stop and the second jaw first stop are engaged with each other, the shuttle can be retainable in the second jaw. When the shuttle second stop and the second jaw first stop are engaged with each other, the shuttle can be passively retained in the second jaw. The shuttle can be passively retainable in the second jaw when the shuttle second stop and the second jaw first stop are engaged with each other. When the shuttle second stop and the second jaw second stop are engaged with each other, the shuttle can be retainable in the second jaw. When the shuttle second stop and the second jaw second stop are engaged with each other, the shuttle can be passively retained in the second jaw. The shuttle can be passively retainable in the second jaw when the shuttle second stop and the second jaw second stop are engaged with each other. The second jaw first stop can be a female stop (e.g., a female stop 416), the second jaw second stop can be a female stop (e.g., a female stop 416), and the shuttle second stop can be a male stop (e.g., a male stop 412). The second jaw first stop can be a male stop (e.g., a male stop 412), the second jaw second stop can be a male stop (e.g., a male stop 412), and the shuttle second stop can be a female stop (e.g., a female stop 416).

The suture passer can have a second jaw (e.g., the second jaw 80). The second jaw can have a second jaw track (e.g., the track 66), a second jaw first stop (e.g., a first female stop 416a), and a second jaw second stop (e.g., a second female stop 416b). The shuttle can be movable in the second jaw track. The shuttle can have a shuttle second stop (e.g., a second male stop 412b). The shuttle second stop can be movable into and out of the second jaw first stop. The shuttle second stop can be movable into and out of the second jaw second stop. The shuttle can be retainable in the second jaw when the shuttle second stop is in the second jaw first stop. The shuttle can be passively retainable in the second jaw when the shuttle second stop is in the second jaw first stop. When the shuttle second stop is in the second jaw first stop, the shuttle can be passively retained in the second jaw. The shuttle can be retainable in the second jaw when the shuttle second stop is in the second jaw second stop. The shuttle can be passively retainable in the second jaw when the shuttle second stop is in the second jaw second stop. When the shuttle second stop is in the second jaw second stop, the shuttle can be passively retained in the second jaw.

The first jaw first stop can be farther from a tip of the first jaw than the first jaw second stop. The second jaw first stop can be farther from a tip of the second jaw than the second jaw second stop.

The shuttle first stop can extend radially away from the shuttle longitudinal axis. The shuttle second stop can extend radially away from the shuttle longitudinal axis. The first jaw first stop can extend radially away from the first jaw track. The first jaw second stop can extend radially away from the first jaw track. The second jaw first stop can extend radially away from the second jaw track. The second jaw second stop can extend radially away from the second jaw track.

The shuttle first stop can extend radially away from the shuttle longitudinal axis. The shuttle second stop can extend laterally away from the shuttle longitudinal axis. The first jaw first stop can extend radially away from the first jaw track. The first jaw second stop can extend radially away from the first jaw track. The second jaw first stop can extend laterally away from the second jaw track. The second jaw second stop can extend laterally away from the second jaw track.

The shuttle first stop can extend laterally away from the shuttle longitudinal axis. The shuttle second stop can extend laterally away from the shuttle longitudinal axis. The first jaw first stop can extend laterally away from the first jaw track. The first jaw second stop can extend laterally away from the first jaw track. The second jaw first stop can extend laterally away from the second jaw track. The second jaw second stop can extend laterally away from the second jaw track.

The first jaw track 64 can be any track in a jaw disclosed, illustrated, and/or contemplated herein.

The second jaw track 66 can be any track in a jaw disclosed, illustrated, and/or contemplated herein.

The first pusher 76 can be any pusher disclosed, illustrated, and/or contemplated herein.

The second pusher 86 can be any pusher disclosed, illustrated, and/or contemplated herein.

The first jaw 78 can be any jaw disclosed, illustrated, and/or contemplated herein.

The second jaw 80 can be any jaw disclosed, illustrated, and/or contemplated herein.

The shuttle 14 can be made from a flexible polymer, such as PEEK, a resilient metal such as Nitinol, any material disclosed herein or combinations thereof. The shuttle 14 can be made from a molded polymer. The shuttle 14 can be pre-curved, for example to reduce resistance when going around curves in the tracks. The shuttle 14 can be made from nickel titanium alloys (e.g., Nitinol), stainless steel, other materials disclosed herein, or combinations thereof.

The shuttle 14 can have a shuttle width The shuttle width can be from about 0.030 in. to about 0.100 in., for example about 0.060 in, or, as another example, about 0.055 in. As another example, the shuttle width can be about 0.053 in. to about 0.057 in., or more broadly, from about 0.35 in. to about 0.075 in., including every 0.001 in. increment within these ranges (e.g., 0.055 in.).

It is apparent to one skilled in the art that various changes and modifications can be made to this disclosure, and equivalents employed, without departing from the spirit and scope of the disclosure. Elements shown with any variation are exemplary for the specific variation and can be used on other variations within this disclosure. Any elements described herein as singular can be pluralized (i.e., anything described as “one” can be more than one). Any species element of a genus element can have the characteristics or elements of any other species element of that genus. The words “may” and “can” are interchangeable (e.g., “may” can be replaced with “can” and “can” can be replaced with “may”). The above-described configurations, elements or complete assemblies and methods and their elements for carrying out the disclosure, and variations of aspects of the disclosure can be combined and modified with each other in any combination. Any phrase involving an “A and/or B” construction or similar construction can mean (1) A alone, (2) B alone, (3) A and B together. Any range disclosed can include any subrange of the range disclosed, for example, a range of 1-10 units can include 2-10 units, 8-10 units, or any other subrange.

We claim:

1. A method of suturing tissue with a suture passer having a shuttle, a first jaw, a second jaw, a first trigger, a second trigger, a grip, and a selector, the method comprising:
selecting a first pusher with the selector;
moving the first jaw toward the second jaw with the first trigger;
moving the first pusher from a first pusher first position to a first pusher second position with the second trigger by actuating the second trigger toward the grip to move the shuttle from the first jaw to the second jaw;
moving the first pusher from the first pusher second position to the first pusher first position with the second trigger; and
moving the first jaw away from the second jaw with the first trigger.

2. The method of claim 1, further comprising:
selecting a second pusher with the selector;
moving the first jaw toward the second jaw with the first trigger;
moving the second pusher from a second pusher first position to a second pusher second position with the second trigger to move the shuttle from the second jaw to the first jaw;
moving the second pusher from the second pusher second position to the second pusher first position with the second trigger; and
moving the first jaw away from the second jaw with the first trigger.

3. The method of claim 2, wherein selecting the first pusher with the selector comprises aligning a mover with a first engager,
wherein moving the first pusher from the first pusher first position to the first pusher second position comprises advancing the first engager with the mover via the second trigger,
wherein selecting the second pusher with the selector comprises aligning the mover with a second engager, and
wherein moving the second pusher from the second pusher first position to the second pusher second position comprises advancing the second engager with the mover via the second trigger.

4. The method of claim 3, wherein the mover is connected to the second trigger, wherein the first engager is connected to the first pusher, and wherein the second engager is connected to the second pusher.

5. The method of claim 4, wherein moving the first pusher from the first pusher second position to the first pusher first position comprises retracting the first engager with the mover via the second trigger, and wherein moving the second pusher from the second pusher second position to the second pusher first position comprises retracting the second engager with the mover via the second trigger.

6. The method of claim 2, wherein moving the first pusher from the first pusher first position to the first pusher second position comprises advancing the first pusher,
wherein moving the second pusher from the second pusher first position to the second pusher second position comprises advancing the second pusher,
wherein moving the first pusher from the first pusher second position to the first pusher first position comprises retracting the first pusher, and
wherein moving the second pusher from the second pusher second position to the second pusher first position comprises retracting the second pusher.

7. The method of claim 1, wherein moving the first jaw toward the second jaw with the first trigger comprises unlocking the second trigger.

8. The method of claim 7, wherein unlocking the second trigger comprises moving a lockout gate from a lockout gate first position to a lockout gate second position with the first trigger, wherein when the lockout gate is in the lockout gate first position, movement of the second trigger is inhibited by the lockout gate, and wherein when the lockout gate is in the lockout gate second position, the second trigger is movable to move the first pusher.

9. The method of claim 1, wherein the suture passer comprises a mover, wherein the mover is lockable and unlockable,
wherein the first jaw comprises a first jaw track and a first jaw first stop,
wherein the shuttle comprises a shuttle longitudinal axis and a shuttle first stop, wherein the shuttle is movable in the first jaw track,
wherein the shuttle first stop extends away from the shuttle longitudinal axis,
wherein the method comprises moving the shuttle first stop into and out of the first jaw first stop, and wherein when the shuttle first stop is in the first jaw first stop, the shuttle is retained in the first jaw.

10. The method of claim 9, further comprising:
moving the mover from a mover first longitudinal position to a mover second longitudinal position; and
releasably locking the mover in the mover first longitudinal position.

11. The method of claim 10, further comprising:
moving the mover from a mover first transverse position to a mover second transverse position; and
releasably locking the mover in the mover first transverse position.

12. The method of claim 11, further comprising releasably locking the mover in the mover second transverse position.

13. The method of claim 9, wherein the second jaw of the suture passer comprises a second jaw track and a second jaw first stop, wherein the shuttle is movable in the second jaw track,
wherein the shuttle further comprises a shuttle second stop,
wherein the shuttle second stop extends away from the shuttle longitudinal axis,
wherein the method comprises the shuttle second stop into and out of the second jaw first stop, and wherein when the shuttle second stop is in the second jaw first stop, the shuttle is retained in the second jaw.

14. The method of claim 13, wherein the suture passer comprises a first jaw second stop and a second jaw second stop, wherein the shuttle first stop is movable into and out of the first jaw second stop, wherein the shuttle second stop is movable into and out of the second jaw second stop, wherein the first jaw second stop is closer to a tip of the first jaw than the first jaw first stop, wherein the second jaw second stop is closer to a tip of the second jaw than the second jaw first stop,
wherein the method comprises:
moving the shuttle first stop into and out of the first jaw second stop; or
moving the shuttle second stop into and out of the second jaw second stop, wherein when the shuttle first stop is in the first jaw second stop, the shuttle is retained in the first jaw, and wherein when the shuttle second stop is in the second jaw second stop, the shuttle is retained in the second jaw.

15. The method of claim 14, wherein the first jaw first stop and the second jaw first stop comprise primary shuttle catches, and wherein the first jaw second stop and the second jaw second stop comprise secondary shuttle catches.

16. The method of claim 1, wherein the first jaw comprises a first jaw track, a first jaw first stop, and a first jaw second stop;
wherein the shuttle comprises a shuttle longitudinal axis and a shuttle first stop, wherein the method comprises:
moving the shuttle in the first jaw track,
engaging the shuttle first stop and the first jaw first stop with each other and/or engaging the shuttle first stop and the first jaw second stop with each other,
wherein when the shuttle first stop and the first jaw first stop are engaged with each other, the shuttle is retained in the first jaw, and
wherein when the shuttle first stop and the first jaw second stop are engaged with each other, the shuttle is retained in the first jaw.

17. The method of claim 16, wherein the first jaw first stop is farther from a tip of the first jaw than the first jaw second stop.

18. The method of claim 16, wherein the second jaw comprises a second jaw track, a second jaw first stop, and a second jaw second stop,
wherein the shuttle comprises a shuttle second stop, wherein the method comprises:
moving the shuttle in the second jaw track,
engaging the shuttle second stop and the second jaw first stop with each other and/or engaging the shuttle second stop and the second jaw second stop with each other,
wherein when the shuttle second stop and the second jaw first stop are engaged with each other, the shuttle is retained in the second jaw, and
wherein when the shuttle second stop and the second jaw second stop are engaged with each other, the shuttle is retained in the second jaw.

19. The method of claim 18, wherein the first jaw first stop is farther from a tip of the first jaw than the first jaw second stop, and wherein the second jaw first stop is farther from a tip of the second jaw than the second jaw second stop.

20. The method of claim 18, wherein the shuttle first stop extends laterally away from the shuttle longitudinal axis, and wherein the shuttle second stop extends laterally away from the shuttle longitudinal axis.

21. The method of claim 20, wherein the first jaw first stop extends laterally away from the first jaw track, wherein the first jaw second stop extends laterally away from the first jaw track, wherein the second jaw first stop extends laterally away from the second jaw track, and wherein the second jaw second stop extends laterally away from the second jaw track.
